SEQUENCE LISTING (299 sequences)

SEQ ID NO: 1 - protein, 22 aa, Artificial sequence
  Synthetic
LCDFGSASHVADNDITPYLVSR

SEQ ID NO: 2 - protein, 10 aa, Artificial sequence
  Synthetic
IGEGTGVVYK

SEQ ID NO: 3 - protein, 11 aa, Artificial sequence
  Synthetic
IGEGTYGVVYK

SEQ ID NO: 4 - protein, 10 aa, Artificial sequence
  Synthetic
IGEGTGVVYK

SEQ ID NO: 5 - protein, 11 aa, Artificial sequence
  Synthetic
IGEGTYGVVYK

SEQ ID NO: 6 - protein, 18 aa, Artificial sequence
  Synthetic
SVLEDFFTATEGQYQPQP

SEQ ID NO: 7 - protein, 10 aa, Artificial sequence
  Synthetic
LIEDNEYTAR

SEQ ID NO: 8 - protein, 16 aa, Artificial sequence
  Synthetic
LIYDFIEDQGGLEAVR

SEQ ID NO: 9 - protein, 10 aa, Artificial sequence
  Synthetic
GEPNVSICSR

SEQ ID NO: 10 - protein, 16 aa, Artificial sequence
  Synthetic
VVQEYIDAFSDYANFK

SEQ ID NO: 11 - protein, 18 aa, Artificial sequence
  Synthetic
DSSSQENMVEDCLYETVK

SEQ ID NO: 12 - protein, 14 aa, Artificial sequence
  Synthetic
ENDYESISDLQQGR

SEQ ID NO: 13 - protein, 20 aa, Artificial sequence
  Synthetic
SGQSLTVPESTTSIQGDPQR

SEQ ID NO: 14 - protein, 18 aa, Artificial sequence
  Synthetic
SVDGDQGLGMEGPYEVLK

SEQ ID NO: 15 - protein, 11 aa, Artificial sequence
  Synthetic
AEFAEYASVDR

SEQ ID NO: 16 - protein, 13 aa, Artificial sequence
  Synthetic
SPSSCNDLYATVK

SEQ ID NO: 17 - protein, 11 aa, Artificial sequence
  Synthetic
AEFAEYASVDR

SEQ ID NO: 18 - protein, 8 aa, Artificial sequence
  Synthetic
IYQYIQSR

SEQ ID NO: 19 - protein, 13 aa, Artificial sequence
  Synthetic
HTDDEMTGYVATR

SEQ ID NO: 20 - protein, 18 aa, Artificial sequence
  Synthetic
AAVPSGASTGIYEALELR

SEQ ID NO: 21 - protein, 13 aa, Artificial sequence
  Synthetic
REEPEALYAAVNK

SEQ ID NO: 22 - protein, 12 aa, Artificial sequence
  Synthetic
EEPEALYAAVNK

SEQ ID NO: 23 - protein, 13 aa, Artificial sequence
  Synthetic
LLAQAEGEPCYIR

SEQ ID NO: 24 - protein, 19 aa, Artificial sequence
  Synthetic
ALPAQVDDPPEPVYANIER

SEQ ID NO: 25 - protein, 11 aa, Artificial sequence
  Synthetic
AVDGYVKPQIK

SEQ ID NO: 26 - protein, 19 aa, Artificial sequence
  Synthetic
VADPDHDHTGFLTEYVATR

SEQ ID NO: 27 - protein, 17 aa, Artificial sequence
  Synthetic
VGWFPANYVEEDYSEYC

SEQ ID NO: 28 - protein, 14 aa, Artificial sequence
  Synthetic
NAEDCLYELPENIR

SEQ ID NO: 29 - protein, 17 aa, Artificial sequence
  Synthetic
ALDVDSGPLENVYESIR

SEQ ID NO: 30 - protein, 15 aa, Artificial sequence
  Synthetic
TEVTPAAQVDVLYSR

SEQ ID NO: 31 - protein, 15 aa, Artificial sequence
  Synthetic
SAFSNLFGGEPLSTR

SEQ ID NO: 32 - protein, 22 aa, Artificial sequence
  Synthetic
SADAPAYQQGQNQLYNELNLGR

SEQ ID NO: 33 - protein, 12 aa, Artificial sequence
  Synthetic
NPQEGLYNELQK

SEQ ID NO: 34 - protein, 13 aa, Artificial sequence
  Synthetic
DTDALHMQALPPR

SEQ ID NO: 35 - protein, 13 aa, Artificial sequence
  Synthetic
KNPQEGLYNELQK

SEQ ID NO: 36 - protein, 15 aa, Artificial sequence
  Synthetic
SGFEGYVELPPIEGR

SEQ ID NO: 37 - protein, 18 aa, Artificial sequence
  Synthetic
TPLTDTSVYTELPNAEPR

SEQ ID NO: 38 - protein, 23 aa, Artificial sequence
  Synthetic
QDSDEQDPQEVTAQLDHCVFIQR

SEQ ID NO: 39 - protein, 15 aa, Artificial sequence
  Synthetic
ELFDDPSVNVQNLDK

SEQ ID NO: 40 - protein, 10 aa, Artificial sequence
  Synthetic
LIEDNEYTAR

SEQ ID NO: 41 - protein, 33 aa, Artificial sequence
  Synthetic
KDPEERPTFEYLQSFLEDYFTATEPQYQPGENL

SEQ ID NO: 42 - protein, 14 aa, Artificial sequence
  Synthetic
VVLPSEEALSLPSK

SEQ ID NO: 43 - protein, 16 aa, Artificial sequence
  Synthetic
TLYDFPGNDAEDLPFK

SEQ ID NO: 44 - protein, 29 aa, Artificial sequence
  Synthetic
AEERPTFDYLQSVLDDFYTATEGQYQQQP

SEQ ID NO: 45 - protein, 22 aa, Artificial sequence
  Synthetic
YCRPESQEHPEADPGSAAPYLK

SEQ ID NO: 46 - protein, 9 aa, Artificial sequence
  Synthetic
DLYSGLNQR

SEQ ID NO: 47 - protein, 15 aa, Artificial sequence
  Synthetic
DGVYDVPLHNPPDAK

SEQ ID NO: 48 - protein, 23 aa, Artificial sequence
  Synthetic
DVYDIPPSHTTQGVYDIPPSSAK

SEQ ID NO: 49 - protein, 23 aa, Artificial sequence
  Synthetic
GPVFSVPVGEIKPQGVYDIPPTK

SEQ ID NO: 50 - protein, 13 aa, Artificial sequence
  Synthetic
GLAESLQPDYSER

SEQ ID NO: 51 - protein, 12 aa, Artificial sequence
  Synthetic
VYENVGLMQQQK

SEQ ID NO: 52 - protein, 14 aa, Artificial sequence
  Synthetic
EISEASENIYSDVR

SEQ ID NO: 53 - protein, 19 aa, Artificial sequence
  Synthetic
SENIYDYLDSSEPAENENK

SEQ ID NO: 54 - protein, 23 aa, Artificial sequence
  Synthetic
SHLSESCSIESDIYAEIPDETLR

SEQ ID NO: 55 - protein, 27 aa, Artificial sequence
  Synthetic
WDSYENLSADGEVLHTQGPVDGSLYAK

SEQ ID NO: 56 - protein, 26 aa, Artificial sequence
  Synthetic
WDSENLSADGEVLHTQGPVDGSLYAK

SEQ ID NO: 57 - protein, 14 aa, Artificial sequence
  Synthetic
LQLDNQYAVLENQK

SEQ ID NO: 58 - protein, 23 aa, Artificial sequence
  Synthetic
KTPQGPPEIYSDTQFPSLQSTAK

SEQ ID NO: 59 - protein, 22 aa, Artificial sequence
  Synthetic
TPQGPPEIYSDTQFPSLQSTAK

SEQ ID NO: 60 - protein, 15 aa, Artificial sequence
  Synthetic
GQEGEYAVPFDAVAR

SEQ ID NO: 61 - protein, 12 aa, Artificial sequence
  Synthetic
TDYIPLLDVDEK

SEQ ID NO: 62 - protein, 13 aa, Artificial sequence
  Synthetic
YATPQVIQAPGPR

SEQ ID NO: 63 - protein, 18 aa, Artificial sequence
  Synthetic
KPTDPVSEDQDPLSSDFK

SEQ ID NO: 64 - protein, 20 aa, Artificial sequence
  Synthetic
SYDYDLIIIGGGSGGLAAAK

SEQ ID NO: 65 - protein, 10 aa, Artificial sequence
  Synthetic
SIYYITGESK

SEQ ID NO: 66 - protein, 11 aa, Artificial sequence
  Synthetic
QVVESAYEVIK

SEQ ID NO: 67 - protein, 16 aa, Artificial sequence
  Synthetic
QETEVELYNEFPEPIK

SEQ ID NO: 68 - protein, 14 aa, Artificial sequence
  Synthetic
QYATLDVYNPFETR

SEQ ID NO: 69 - protein, 15 aa, Artificial sequence
  Synthetic
IHFPLATAPVISAEK

SEQ ID NO: 70 - protein, 20 aa, Artificial sequence
  Synthetic
YAGSALQYEDVSTAVQNLQK

SEQ ID NO: 71 - protein, 10 aa, Artificial sequence
  Synthetic
IGEGTGTVFK

SEQ ID NO: 72 - protein, 25 aa, Artificial sequence
  Synthetic
LQPFHSTELEDDAIYSVHVPAGLYR

SEQ ID NO: 73 - protein, 12 aa, Artificial sequence
  Synthetic
LLPLDKDYYVVR

SEQ ID NO: 74 - protein, 13 aa, Artificial sequence
  Synthetic
HADAEMTGYVVTR

SEQ ID NO: 75 - protein, 19 aa, Artificial sequence
  Synthetic
IADPEHDHTGFLTEYVATR

SEQ ID NO: 76 - protein, 8 aa, Artificial sequence
  Synthetic
IYEPLDVK

SEQ ID NO: 77 - protein, 10 aa, Artificial sequence
  Synthetic
IIQLLDDYPK

SEQ ID NO: 78 - protein, 15 aa, Artificial sequence
  Synthetic
ENDTITIYSTINHSK

SEQ ID NO: 79 - DNA, 36 bp, Homo sapiens
cgcggacacagacgccatagaggtacacgatgtgtt

SEQ ID NO: 80 - DNA, 20 bp, Homo sapiens
cgcggacacagacgccaaag

SEQ ID NO: 81 - DNA, 21 bp, Homo sapiens
cgcggacacagacgccaaaga

SEQ ID NO: 82 - DNA, 33 bp, Homo sapiens
cgcggacacagacgccaaagaggtacacgatgt

SEQ ID NO: 83 - DNA, 36 bp, Homo sapiens
cgcggacacagacgccaaagaggtacacgatgtgtt

SEQ ID NO: 84 - DNA, 36 bp, Homo sapiens
cgcggacacagacgccatagcgggacacgatgtgtt

SEQ ID NO: 85 - DNA, 35 bp, Homo sapiens
gcggacacagacgccaaagaggtacacgatgtgtt

SEQ ID NO: 86 - DNA, 36 bp, Homo sapiens
cttggctcatcaagctcgctgcttccaggaatgact

SEQ ID NO: 87 - DNA, 36 bp, Homo sapiens
cttggctcatcaagctcactgcttccaggaatgact

SEQ ID NO: 88 - DNA, 35 bp, Homo sapiens
ttggctcatcaagctcactgcttccaggaatgact

SEQ ID NO: 89 - DNA, 20 bp, Homo sapiens
tgtacctctatggcgtctgt

SEQ ID NO: 90 - DNA, 17 bp, Homo sapiens
ggaagcagtgagcttga

SEQ ID NO: 91 - DNA, 24 bp, Homo sapiens
atatgatctgcgagtgaatgagat

SEQ ID NO: 92 - DNA, 22 bp, Homo sapiens
gatcgctgtcgtctgggattat

SEQ ID NO: 93 - DNA, 21 bp, Homo sapiens
acacgttcagttactttgttg

SEQ ID NO: 94 - DNA, 33 bp, Homo sapiens
tttgaaagagaacacacttactcgtctcggaca

SEQ ID NO: 95 - DNA, 32 bp, Homo sapiens
ttgaaagagattactctccagtcgcggacaca

SEQ ID NO: 96 - DNA, 33 bp, Homo sapiens
ttgaaagagattactctccacgtcccggacaca

SEQ ID NO: 97 - DNA, 33 bp, Homo sapiens
ttgaaagagaacacacttactctcacggaaaca

SEQ ID NO: 98 - DNA, 33 bp, Homo sapiens; identical to SEQ ID NO: 97

SEQ ID NO: 99 - DNA, 33 bp, Homo sapiens
ttgaaagagattactctccacagaacggaaaca

SEQ ID NO: 100 - DNA, 33 bp, Homo sapiens; identical to SEQ ID NO: 97

SEQ ID NO: 101 - DNA, 33 bp, Homo sapiens
ttgaaagagattactctccacgtcacggaaaca

SEQ ID NO: 102 - DNA, 33 bp, Homo sapiens
tgaaagagaatactctccacgtcacggaaacag

SEQ ID NO: 103 - DNA, 34 bp, Homo sapiens
gaaagagaactactctccacgtcacggacacaga

SEQ ID NO: 104 - DNA, 32 bp, Homo sapiens
gaaagagaacacttactctccacggacacaga

SEQ ID NO: 105 - DNA, 33 bp, Homo sapiens
gaaagagaacacacttactctcccggacacaga

SEQ ID NO: 106 - DNA, 33 bp, Homo sapiens
gaaagagaacacacttactctcccggaaacaga

SEQ ID NO: 107 - DNA, 33 bp, Homo sapiens; identical to SEQ ID NO: 106

SEQ ID NO: 108 - DNA, 32 bp, Homo sapiens
gaaagagaacacttactctccgcggaaacaga

SEQ ID NO: 109 - DNA, 33 bp, Homo sapiens
aaagagaacacacttactcgcctcggacacaga

SEQ ID NO: 110 - DNA, 33 bp, Homo sapiens
aaagagaacacacttactctccacggacacaga

SEQ ID NO: 111 - DNA, 33 bp, Homo sapiens
aaagagaacacacttactctcctcggacacaga

SEQ ID NO: 112 - DNA, 33 bp, Homo sapiens
aaagagaacacacttactctccacggaaacaga

SEQ ID NO: 113 - DNA, 34 bp, Homo sapiens
aaagagaacacacttactctccacggaaacagac

SEQ ID NO: 114 - DNA, 33 bp, Homo sapiens
aagagaacacacttactctccacggaaacagac

SEQ ID NO: 115 - DNA, 33 bp, Homo sapiens
agagaacacacttactctccacgacacagacgc

SEQ ID NO: 116 - DNA, 33 bp, Homo sapiens
gagaacacactccacgtcgcggacacagacgcc

SEQ ID NO: 117 - DNA, 33 bp, Homo sapiens
gagaacacacttactctccacgtcacagacgcc

SEQ ID NO: 118 - DNA, 33 bp, Homo sapiens
gagaacccactccacgtcacgacacagacgcca

SEQ ID NO: 119 - DNA, 33 bp, Homo sapiens
agaacacacttccacgtcgcggacagagacgcc

SEQ ID NO: 120 - DNA, 33 bp, Homo sapiens
agaacacactccacgtcacggaaacagacgcca

SEQ ID NO: 121 - DNA, 33 bp, Homo sapiens
cacacttactcgtcacggaagacgccatagagg

SEQ ID NO: 122 - DNA, 33 bp, Homo sapiens
cacttactcttcgcggacacagacgccatagag

SEQ ID NO: 123 - DNA, 33 bp, Homo sapiens
cacttactctccacgtcgcggacgccatagagg

SEQ ID NO: 124 - DNA, 33 bp, Homo sapiens
cacttactcttcacggaaacgacgccatagagg

SEQ ID NO: 125 - DNA, 33 bp, Homo sapiens
cacttattcttcacggaaacgacgccatagagg

SEQ ID NO: 126 - DNA, 33 bp, Homo sapiens
acttagtctccacgtcgcggacaccatagaggt

SEQ ID NO: 127 - DNA, 33 bp, Homo sapiens
acttactctccacgtcgcggacacatagaggta

SEQ ID NO: 128 - DNA, 34 bp, Homo sapiens
acttactctccacgtcgcggacaccatagaggta

SEQ ID NO: 129 - DNA, 33 bp, Homo sapiens
cttactctccacggacacagacgccatagaggt

SEQ ID NO: 130 - DNA, 32 bp, Homo sapiens
ttactctccagtcgcggacacacatagaggta

SEQ ID NO: 131 - DNA, 32 bp, Homo sapiens
ttactctacacggcggacacagtagaggtaca

SEQ ID NO: 132 - DNA, 32 bp, Homo sapiens
tactctccacggacacagacgccatagaggta

SEQ ID NO: 133 - DNA, 33 bp, Homo sapiens
tactctccacacacagacgccatagaggtacac

SEQ ID NO: 134 - DNA, 33 bp, Homo sapiens; identical to SEQ ID NO: 133

SEQ ID NO: 135 - DNA, 32 bp, Homo sapiens
tactctccacgacacagacgatagaggtacac

SEQ ID NO: 136 - DNA, 33 bp, Homo sapiens; identical to SEQ ID NO: 133

SEQ ID NO: 137 - DNA, 33 bp, Homo sapiens
actctccacgtcacggacacagatagaggtaca

SEQ ID NO: 138 - DNA, 33 bp, Homo sapiens
actctccacggacacagacgccatagaggtaca

SEQ ID NO: 139 - DNA, 33 bp, Homo sapiens
actctccacgtcacggacacagaagaggtacac

SEQ ID NO: 140 - DNA, 33 bp, Homo sapiens; identical to SEQ ID NO: 139

SEQ ID NO: 141 - DNA, 33 bp, Homo sapiens
actctccacgacacagacgccatagaggtacac

SEQ ID NO: 142 - DNA, 33 bp, Homo sapiens
actctccacggcacagacgccatagaggtacac

SEQ ID NO: 143 - DNA, 33 bp, Homo sapiens
ctctccacggacacagacgccatagaggtacac

SEQ ID NO: 144 - DNA, 33 bp, Homo sapiens
ctctccacgtacacagacgccatagaggtacac

SEQ ID NO: 145 - DNA, 33 bp, Homo sapiens
tctcctcgtcacgcagacgccatagaggtacac

SEQ ID NO: 146 - DNA, 33 bp, Homo sapiens
tctccacgtcacggaaacagacggaggtacacg

SEQ ID NO: 147 - DNA, 33 bp, Homo sapiens
tctccacgtcgcggacacagacgaggtacacga

SEQ ID NO: 148 - DNA, 33 bp, Homo sapiens
tctccacgccacggaaacagacgaggtacacga

SEQ ID NO: 149 - DNA, 33 bp, Homo sapiens
tctccacgtcacggaaacagacgaggtacacga

SEQ ID NO: 150 - DNA, 33 bp, Homo sapiens; identical to SEQ ID NO: 149

SEQ ID NO: 151 - DNA, 33 bp, Homo sapiens; identical to SEQ ID NO: 149

SEQ ID NO: 152 - DNA, 33 bp, Homo sapiens
ctccacgtcgcggacacagacgcggtacacgat

SEQ ID NO: 153 - DNA, 33 bp, Homo sapiens
cacgtcgcggacacagacgccatacacgatgtg

SEQ ID NO: 154 - DNA, 33 bp, Homo sapiens
cacgtcacggaaacagacgccatacacgatgtg

SEQ ID NO: 155 - DNA, 33 bp, Homo sapiens; identical to SEQ ID NO: 154

SEQ ID NO: 156 - DNA, 32 bp, Homo sapiens
gtcacggaaacaacgccatagaacgatgtgtt

SEQ ID NO: 157 - DNA, 33 bp, Homo sapiens
gtcgcggacacagacgccatagacgatgtgttt

SEQ ID NO: 158 - DNA, 33 bp, Homo sapiens
gtcacggacacagacgccatagacgatgtgttt

SEQ ID NO: 159 - DNA, 33 bp, Homo sapiens; identical to SEQ ID NO: 158

SEQ ID NO: 160 - DNA, 33 bp, Homo sapiens
tcgcggacacagacgccatagaggatgtgtttg

SEQ ID NO: 161 - DNA, 33 bp, Homo sapiens
tcgcggacaccatagaggtacacgatgggtttg

SEQ ID NO: 162 - DNA, 33 bp, Homo sapiens
tcacggaaaccatagaggtacacgatgtgtttg

SEQ ID NO: 163 - DNA, 33 bp, Homo sapiens
gcggacacagacgccatagaggttgtgtttgtg

SEQ ID NO: 164 - DNA, 33 bp, Homo sapiens; identical to SEQ ID NO: 163

SEQ ID NO: 165 - DNA, 33 bp, Homo sapiens; identical to SEQ ID NO: 163

SEQ ID NO: 166 - DNA, 33 bp, Homo sapiens; identical to SEQ ID NO: 163

SEQ ID NO: 167 - DNA, 33 bp, Homo sapiens
acggacacagacgccatagaggttgtgtttgtg

SEQ ID NO: 168 - DNA, 33 bp, Homo sapiens; identical to SEQ ID NO: 167

SEQ ID NO: 169 - DNA, 33 bp, Homo sapiens
acggacacagtagaggtacacgatgtgtttgtg

SEQ ID NO: 170 - DNA, 33 bp, Homo sapiens; identical to SEQ ID NO: 167

SEQ ID NO: 171 - DNA, 33 bp, Homo sapiens
acggaaacagcagggtacacgatgtgtttgtgg

SEQ ID NO: 172 - DNA, 33 bp, Homo sapiens
aggaaacagaagaggtacacgatgtgtttgtgg

SEQ ID NO: 173 - DNA, 33 bp, Homo sapiens
cggaaacagaagaggtacacatgtgtttgtggg

SEQ ID NO: 174 - DNA, 33 bp, Homo sapiens
ggacacagacgccatagaggtactgtttgtggg

SEQ ID NO: 175 - DNA, 33 bp, Homo sapiens
ggacacagacgaggtacacgatgtgtttgtggg

SEQ ID NO: 176 - DNA, 33 bp, Homo sapiens
gacacagacggaggtacacgatgtgtttgtggg

SEQ ID NO: 177 - DNA, 32 bp, Homo sapiens
gacacagacgccagaggtacacgtttgttgga

SEQ ID NO: 178 - DNA, 33 bp, Homo sapiens
gacacagacgccatagaggtacagtttgtggga

SEQ ID NO: 179 - DNA, 33 bp, Homo sapiens; identical to SEQ ID NO: 178

SEQ ID NO: 180 - DNA, 33 bp, Homo sapiens
gacacagacgaggtacacgatgtgtttgtggga

SEQ ID NO: 181 - DNA, 33 bp, Homo sapiens
gaaacagacgagttacacgagtgtttgtgggag

SEQ ID NO: 182 - DNA, 33 bp, Homo sapiens
cacagacgccggtacacgatgtgtttgtgggag

SEQ ID NO: 183 - DNA, 33 bp, Homo sapiens
cacagacgccgtacacgatgtgtttgtgggaga

SEQ ID NO: 184 - DNA, 33 bp, Homo sapiens
cacagacgccgtacgcgatgtgtttgtgggaga

SEQ ID NO: 185 - DNA, 33 bp, Homo sapiens
aacagacgccatagaggtacacgttgtgggagt

SEQ ID NO: 186 - DNA, 32 bp, Homo sapiens
cacagacgccgtacacgatgttgtgggagacc

SEQ ID NO: 187 - DNA, 33 bp, Homo sapiens
ggaccattatggacatcaggacaccaagtagct

SEQ ID NO: 188 - DNA, 33 bp, Homo sapiens; identical to SEQ ID NO: 187

SEQ ID NO: 189 - DNA, 33 bp, Homo sapiens
ggaccattatcaggacattctcaccaagtagct

SEQ ID NO: 190 - DNA, 33 bp, Homo sapiens; identical to SEQ ID NO: 187

SEQ ID NO: 191 - DNA, 33 bp, Homo sapiens
gaaccattatcaggacattctcaccaagtagat

SEQ ID NO: 192 - DNA, 32 bp, Homo sapiens
gaccatatggacatcaggacatccaagtagct

SEQ ID NO: 193 - DNA, 33 bp, Homo sapiens
gaccatgatgcaggacattccaccaagtagctc

SEQ ID NO: 194 - DNA, 33 bp, Homo sapiens
accattatggaggacattctcaccaagtagctc

SEQ ID NO: 195 - DNA, 33 bp, Homo sapiens; identical to SEQ ID NO: 194

SEQ ID NO: 196 - DNA, 33 bp, Homo sapiens
accattatggacatcaggacattaagtagctca

SEQ ID NO: 197 - DNA, 33 bp, Homo sapiens
accattatggacatcaggacattaagtagatca

SEQ ID NO: 198 - DNA, 34 bp, Homo sapiens
accattatggggacattctcaccaagtagctcag

SEQ ID NO: 199 - DNA, 32 bp, Homo sapiens
ccattatggacaaggacattctgtagctcagg

SEQ ID NO: 200 - DNA, 33 bp, Homo sapiens
cattatggacatcaggacattctaatagatcag

SEQ ID NO: 201 - DNA, 33 bp, Homo sapiens
cattatggacatcaggacattctgtagctcagg

SEQ ID NO: 202 - DNA, 33 bp, Homo sapiens
cattatggacacattctcaccaagtagctcagg

SEQ ID NO: 203 - DNA, 33 bp, Homo sapiens
cattatggacacattctcaccaagtagatcagg

SEQ ID NO: 204 - DNA, 33 bp, Homo sapiens
aatagggacaacattctcaccaagtagctcagg

SEQ ID NO: 205 - DNA, 33 bp, Homo sapiens
attatggacaacattctcaccaagtagctcagg

SEQ ID NO: 206 - DNA, 33 bp, Homo sapiens
ttatggacatactattctcaccaagtagctcag

SEQ ID NO: 207 - DNA, 33 bp, Homo sapiens
ttatggacatacattctcaccaagtagctcagg

SEQ ID NO: 208 - DNA, 33 bp, Homo sapiens
ttatggacatcaggacattctcatagatcaggg

SEQ ID NO: 209 - DNA, 34 bp, Homo sapiens
ttatggacatcaggacattctcacgatcagggca

SEQ ID NO: 210 - DNA, 33 bp, Homo sapiens
tatggacatcaggacattctcacgctcagggca

SEQ ID NO: 211 - DNA, 33 bp, Homo sapiens
tatggacatcaggacattctcacgatcagggca

SEQ ID NO: 212 - DNA, 33 bp, Homo sapiens; identical to SEQ ID NO: 211

SEQ ID NO: 213 - DNA, 33 bp, Homo sapiens
tatggacatctctcaccaagtagctcagggcac

SEQ ID NO: 214 - DNA, 32 bp, Homo sapiens
atggacatcaggcattctcaccgatcagggca

SEQ ID NO: 215 - DNA, 32 bp, Homo sapiens
atggacatcaggcattctcaccatcagggcac

SEQ ID NO: 216 - DNA, 34 bp, Homo sapiens
gacatcaggacaccaagtagctcagggcactggg

SEQ ID NO: 217 - DNA, 33 bp, Homo sapiens
catcaggacaccaagtagctcagggcattggcc

SEQ ID NO: 218 - DNA, 33 bp, Homo sapiens
catcaggacaccaagtagctcagggcactggcc

SEQ ID NO: 219 - DNA, 33 bp, Homo sapiens; identical to SEQ ID NO: 218

SEQ ID NO: 220 - DNA, 33 bp, Homo sapiens; identical to SEQ ID NO: 218

SEQ ID NO: 221 - DNA, 33 bp, Homo sapiens
catcaggacaccaagtagatcagggcactggcc

SEQ ID NO: 222 - DNA, 33 bp, Homo sapiens; identical to SEQ ID NO: 221

SEQ ID NO: 223 - DNA, 33 bp, Homo sapiens
atcaggacatccaagtagctcagggcactggcc

SEQ ID NO: 224 - DNA, 33 bp, Homo sapiens
atcaggacatcaagtagctcagggcactggcca

SEQ ID NO: 225 - DNA, 33 bp, Homo sapiens
atcaggacatcaagtagatcagggcactggcca

SEQ ID NO: 226 - DNA, 33 bp, Homo sapiens; identical to SEQ ID NO: 225

SEQ ID NO: 227 - DNA, 33 bp, Homo sapiens
caggacattctcaccaagtagctcactggccag

SEQ ID NO: 228 - DNA, 33 bp, Homo sapiens
caggacattctcaccaagtagatcactggccag

SEQ ID NO: 229 - DNA, 33 bp, Homo sapiens
caggacattcagtagctcagggcactggccagc

SEQ ID NO: 230 - DNA, 33 bp, Homo sapiens
caggacattctcaccaagtagctctggccagct

SEQ ID NO: 231 - DNA, 33 bp, Homo sapiens
caggacattctcaccaagtagatctggccagct

SEQ ID NO: 232 - DNA, 33 bp, Homo sapiens
aggacattctagtagctcagggcactggccagc

SEQ ID NO: 233 - DNA, 33 bp, Homo sapiens
aggacattctgtagctcagggcactggccagct

SEQ ID NO: 234 - DNA, 33 bp, Homo sapiens
aggacattctcaccaagtagctcctggccagct

SEQ ID NO: 235 - DNA, 33 bp, Homo sapiens
aggacattcttagctcagggcactggtaagctg

SEQ ID NO: 236 - DNA, 33 bp, Homo sapiens
ggacattctcaccaagtagctcatggccagctg

SEQ ID NO: 237 - DNA, 33 bp, Homo sapiens
gacattctcatagctcagggcactggccagctg

SEQ ID NO: 238 - DNA, 33 bp, Homo sapiens
gacattctcaccaagtagctcagtggccagctg

SEQ ID NO: 239 - DNA, 33 bp, Homo sapiens
gacattctccagctcagggcactggccagctgt

SEQ ID NO: 240 - DNA, 33 bp, Homo sapiens
gacattctcaagatcagggcactggccagctgt

SEQ ID NO: 241 - DNA, 33 bp, Homo sapiens
gacattctcaagctcagggctggccagctgttt

SEQ ID NO: 242 - DNA, 33 bp, Homo sapiens
acattctcaccaagtagctcagggccagctgtt

SEQ ID NO: 243 - DNA, 33 bp, Homo sapiens
cattctcaccgatcagggcactggccagctgtt

SEQ ID NO: 244 - DNA, 33 bp, Homo sapiens; identical to SEQ ID NO: 243

SEQ ID NO: 245 - DNA, 33 bp, Homo sapiens
cattcccaccctcagggcactggccagctgttt

SEQ ID NO: 246 - DNA, 33 bp, Homo sapiens
cattctcaccctcagggcactggcctgctgttt

SEQ ID NO: 247 - DNA, 32 bp, Homo sapiens
attctagccaagtagctcagggccagctgttt

SEQ ID NO: 248 - DNA, 33 bp, Homo sapiens
attctcaccaagtagctcagggtcagctgtttg

SEQ ID NO: 249 - DNA, 33 bp, Homo sapiens
attctcaccaagtagctcagggccagctgtttg

SEQ ID NO: 250 - DNA, 33 bp, Homo sapiens; identical to SEQ ID NO: 249

SEQ ID NO: 251 - DNA, 33 bp, Homo sapiens; identical to SEQ ID NO: 249

SEQ ID NO: 252 - DNA, 33 bp, Homo sapiens
ttctcaccaagtagctcagggcaagctgtttgg

SEQ ID NO: 253 - DNA, 33 bp, Homo sapiens
tctcaccaagtagctcagggcacagctgtttgg

SEQ ID NO: 254 - DNA, 33 bp, Homo sapiens
tctcaccaagtagatcagggcacagctgtttgg

SEQ ID NO: 255 - DNA, 33 bp, Homo sapiens; identical to SEQ ID NO: 254

SEQ ID NO: 256 - DNA, 33 bp, Homo sapiens
tctcaccaagtagatcagggcacgctgtttggc

SEQ ID NO: 257 - DNA, 33 bp, Homo sapiens; identical to SEQ ID NO: 256

SEQ ID NO: 258 - DNA, 33 bp, Homo sapiens; identical to SEQ ID NO: 256

SEQ ID NO: 259 - DNA, 33 bp, Homo sapiens; identical to SEQ ID NO: 256

SEQ ID NO: 260 - DNA, 33 bp, Homo sapiens; identical to SEQ ID NO: 256

SEQ ID NO: 261 - DNA, 33 bp, Homo sapiens
ctcaccaagtagctcagggcactgctgtttggc

SEQ ID NO: 262 - DNA, 33 bp, Homo sapiens
tcaccaagtagctcagggcactgtgtttggcag

SEQ ID NO: 263 - DNA, 34 bp, Homo sapiens
caccaagtagctcagggcactggctttggcaact

SEQ ID NO: 264 - DNA, 33 bp, Homo sapiens
accaagtagatcagggcactggcgtttggcaac

SEQ ID NO: 265 - DNA, 33 bp, Homo sapiens
accaagtagccactggccagctgtttggcaact

SEQ ID NO: 266 - DNA, 33 bp, Homo sapiens
accaagtagatcagggcactggctttggcaact

SEQ ID NO: 267 - DNA, 33 bp, Homo sapiens
ccaagtagatcactggccagctgtttggcaact

SEQ ID NO: 268 - DNA, 33 bp, Homo sapiens
caagtagctcactggccagctgtttggcaactt

SEQ ID NO: 269 - DNA, 33 bp, Homo sapiens
caagtagctcagggcactggccattggcaactt

SEQ ID NO: 270 - DNA, 33 bp, Homo sapiens; identical to SEQ ID NO: 268

SEQ ID NO: 271 - DNA, 33 bp, Homo sapiens
caagtagatcagggcactggccattggcaactt

SEQ ID NO: 272 - DNA, 33 bp, Homo sapiens
caagtagctcagggcactggccatggcaacttt

SEQ ID NO: 273 - DNA, 33 bp, Homo sapiens
aagtagctcatggccagctgtttggcaactttg

SEQ ID NO: 274 - DNA, 33 bp, Homo sapiens
agtagctcagtggccagctgtttggcaactttg

SEQ ID NO: 275 - DNA, 33 bp, Homo sapiens; identical to SEQ ID NO: 274

SEQ ID NO: 276 - DNA, 34 bp, Homo sapiens
gtagctcagggcactggccagctgcaactttgaa

SEQ ID NO: 277 - DNA, 33 bp, Homo sapiens
tagatcagggcactggccagctgaactttgaat

SEQ ID NO: 278 - DNA, 33 bp, Homo sapiens
tagctcagggcagctgtttggcaactttgaatt

SEQ ID NO: 279 - DNA, 33 bp, Homo sapiens; identical to SEQ ID NO: 278

SEQ ID NO: 280 - DNA, 33 bp, Homo sapiens
agctcagggccagctgtttggcaactttgaatt

SEQ ID NO: 281 - DNA, 33 bp, Homo sapiens
agctcagggcactggccagctgtactttgaatt

SEQ ID NO: 282 - DNA, 33 bp, Homo sapiens
gctcagggctcgctgtttggcaactttgaattt

SEQ ID NO: 283 - DNA, 33 bp, Homo sapiens
gctcagggcactggccagctgttctttgaattt

SEQ ID NO: 284 - DNA, 33 bp, Homo sapiens
gatcagggcaagctgtttggcaactttgaattt

SEQ ID NO: 285 - DNA, 33 bp, Homo sapiens
ctcagggcacgctgtttggcaactttgaatttc

SEQ ID NO: 286 - protein, 51 aa, Homo sapiens
DSEIGGITIAWKFDSPERNLWNLKPFTTRDFSIRSLADRLGDLSYLIYVFP

SEQ ID NO: 287 - protein, 51 aa, Homo sapiens
DSEIGGITIAWKFDSQERMFWNLMPFTTRDFSIRSLADRLGDLNYLIYVFP

SEQ ID NO: 288 - protein, 86 aa, Homo sapiens
FHKIRNEDLIFNESLGQGTFTKIFKGVRREVGDYGQLHETEVLLKVLDKAHRNYSESFFEAASMMSKLSHKHLVLNYGVCVCGDEN

SEQ ID NO: 289 - protein, 89 aa, Homo sapiens
FDRILKKDLVQGEHLGRGTRTHIYSGTLMDYKDDEGTSEEKKIKVILKVLDPSHRDISLAFFEAASMMRQVSHKHIVYLYGVCVRDVEN

SEQ ID NO: 290 - protein, 89 aa, Homo sapiens
QMVFHKIRNEDLIFNESLGQGTFTKIFKGVRREVGDYGQLHETEVLLKVLDKAHRNYSESFFEAASMMSKLSHKHLVLNYGVCVCGDEN

SEQ ID NO: 291 - protein, 87 aa, Homo sapiens
QMTFHKIPADSLEWHENLGHGSFTKIYRGCRHEVVDGEARKTEVLLKVMDAKHKNCMESFLEAASLMSQVSYRHLVLLHGVCMAGDS

SEQ ID NO: 292 - DNA, 5053 bp, Homo sapiens
tgcagacagtgcgggcctgcgcccagtcccggctgtcctcgccgcgacccctcctcagccctgggcgcgcgcacgctggggccccgcggggctggccgcctagcgagcctgccggtcgaccccagccagcgcagcgacggggcgctgcctggcccaggcgcacacggaagtgcgcttctctgaagtagctttggaaagtagagaagaaaatccagtttgcttcttggagaacactggacagctgaataaatgcagtatctaaatataaaagaggactgcaatgccatggctttctgtgctaaaatgaggagctccaagaagactgaggtgaacctggaggcccctgagccaggggtggaagtgatcttctatctgtcggacagggagcccctccggctgggcagtggagagtacacagcagaggaactgtgcatcagggctgcacaggcatgccgtatctctcctctttgtcacaacctctttgccctgtatgacgagaacaccaagctctggtatgctccaaatcgcaccatcaccgttgatgacaagatgtccctccggctccactaccggatgaggttctatttcaccaattggcatggaaccaacgacaatgagcagtcagtgtggcgtcattctccaaagaagcagaaaaatggctacgagaaaaaaaagattccagatgcaacccctctccttgatgccagctcactggagtatctgtttgctcagggacagtatgatttggtgaaatgcctggctcctattcgagaccccaagaccgagcaggatggacatgatattgagaacgagtgtctagggatggctgtcctggccatctcacactatgccatgatgaagaagatgcagttgccagaactgcccaaggacatcagctacaagcgatatattccagaaacattgaataagtccatcagacagaggaaccttctcaccaggatgcggataaataatgttttcaaggatttcctaaaggaatttaacaacaagaccatttgtgacagcagcgtgtccacgcatgacctgaaggtgaaatacttggctaccttggaaactttgacaaaacattacggtgctgaaatatttgagacttccatgttactgatttcatcagaaaatgagatgaattggtttcattcgaatgacggtggaaacgttctctactacgaagtgatggtgactgggaatcttggaatccagtggaggcataaaccaaatgttgtttctgttgaaaaggaaaaaaataaactgaagcggaaaaaactggaaaataaacacaagaaggatgaggagaaaaacaagatccgggaagagtggaacaatttttcttacttccctgaaatcactcacattgtaataaaggagtctgtggtcagcattaacaagcaggacaacaagaaaatggaactgaagctctcttcccacgaggaggccttgtcctttgtgtccctggtagatggctacttccggctcacagcagatgcccatcattacctctgcaccgacgtggcccccccgttgatcgtccacaacatacagaatggctgtcatggtccaatctgtacagaatacgccatcaataaattgcggcaagaaggaagcgaggaggggatgtacgtgctgaggtggagctgcaccgactttgacaacatcctcatgaccgtcacctgctttgagaagtctgagcaggtgcagggtgcccagaagcagttcaagaactttcagatcgaggtgcagaagggccgctacagtctgcacggttcggaccgcagcttccccagcttgggagacctcatgagccacctcaagaagcagatcctgcgcacggataacatcagcttcatgctaaaacgctgctgccagcccaagccccgagaaatctccaacctgctggtggctactaagaaagcccaggagtggcagcccgtctaccccatgagccagctgagtttcgatcggatcctcaagaaggatctggtgcagggcgagcaccttgggagaggcacgagaacacacatctattctgggaccctgatggattacaaggatgacgaaggaacttctgaagagaagaagataaaagtgatcctcaaagtcttagaccccagccacagggatatttccctggccttcttcgaggcagccagcatgatgagacaggtctcccacaaacacatcgtgtacctctatggcgtctgtgtccgcgacgtggagaatatcatggtggaagagtttgtggaagggggtcctctggatctcttcatgcaccggaaaagcgatgtccttaccacaccatggaaattcaaagttgccaaacagctggccagtgccctgagctacttggaggataaagacctggtccatggaaatgtgtgtactaaaaacctcctcctggcccgtgagggcatcgacagtgagtgtggcccattcatcaagctcagtgaccccggcatccccattacggtgctgtctaggcaagaatgcattgaacgaatcccatggattgctcctgagtgtgttgaggactccaagaacctgagtgtggctgctgacaagtggagctttggaaccacgctctgggaaatctgctacaatggcgagatccccttgaaagacaagacgctgattgagaaagagagattctatgaaagccggtgcaggccagtgacaccatcatgtaaggagctggctgacctcatgacccgctgcatgaactatgaccccaatcagaggcctttcttccgagccatcatgagagacattaataagcttgaagagcagaatccagatattgtttcagaaaaaaaaccagcaactgaagtggaccccacacattttgaaaagcgcttcctaaagaggatccgtgacttgggagagggccactttgggaaggttgagctctgcaggtatgaccccgaaggggacaatacaggggagcaggtggctgttaaatctctgaagcctgagagtggaggtaaccacatagctgatctgaaaaaggaaatcgagatcttaaggaacctctatcatgagaacattgtgaagtacaaaggaatctgcacagaagacggaggaaatggtattaagctcatcatggaatttctgccttcgggaagccttaaggaatatcttccaaagaataagaacaaaataaacctcaaacagcagctaaaatatgccgttcagatttgtaaggggatggactatttgggttctcggcaatacgttcaccgggacttggcagcaagaaatgtccttgttgagagtgaacaccaagtgaaaattggagacttcggtttaaccaaagcaattgaaaccgataaggagtattacaccgtcaaggatgaccgggacagccctgtgttttggtatgctccagaatgtttaatgcaatctaaattttatattgcctctgacgtctggtcttttggagtcactctgcatgagctgctgacttactgtgattcagattctagtcccatggctttgttcctgaaaatgataggcccaacccatggccagatgacagtcacaagacttgtgaatacgttaaaagaaggaaaacgcctgccgtgcccacctaactgtccagatgaggtttatcaacttatgaggaaatgctgggaattccaaccatccaatcggacaagctttcagaaccttattgaaggatttgaagcacttttaaaataagaagcatgaataacatttaaattccacagattatcaagtccttctcctgcaacaaatgcccaagtcattttttaaaaatttctaatgaaagaagtttgtgttctgtccaaaaagtcactgaactcatacttcagtacatatacatgtataaggcacactgtagtgcttaatatgtgtaaggacttcctctttaaatttggtaccagtaacttagtgacacataatgacaaccaaaatatttgaaagcacttaagcactcctccttgtggaaagaatataccaccatttcatctggctagttcaccatcacaactgcattaccaaaaggggatttttgaaaacgaggagttgaccaaaataatatctgaagatgattgcttttccctgctgccagctgatctgaaatgttttgctggcacattaatcatagataaagaaagattgatggacttagccctcaaatttcagtatctatacagtactagaccatgcattcttaaaatattagataccaggtagtatatattgtttctgtacaaaaatgactgtattctctcaccagtaggacttaaactttgtttctccagtggcttagctcctgttcctttgggtgatcactagcacccatttttgagaaagctggttctacatggggggatagctgtggaatagataatttgctgcatgttaattctcaagaactaagcctgtgccagtgctttcctaagcagtatacctttaatcagaactcattcccagaacctggatgctattacacatgcttttaagaaacgtcaatgtatatccttttataactctaccactttggggcaagctattccagcactggttttgaatgctgtatgcaaccagtctgaataccacatacgctgcactgttcttagagggtttccatacttaccaccgatctacaagggttgatccctgtttttaccatcaatcatcaccctgtggtgcaacacttgaaagacccggctagaggcactatggacttcaggatccactagacagttttcagtttgcttggaggtagctgggtaatcaaaaatgtttagtcattgattcaatgtgaacgattacggtctttatgaccaagagtctgaaaatctttttgttatgctgtttagtattcgtttgatattgttacttttcacctgttgagcccaaattcaggattggttcagtggcagcaatgaagttgccatttaaatttgttcatagcctacatcaccaaggtctctgtgtcaaacctgtggccactctatatgcactttgtttactctttatacaaataaatatactaaagactttacatgca

SEQ ID NO: 293 - protein, 1154 aa, Homo sapiens
MQYLNIKEDCNAMAFCAKMRSSKKTEVNLEAPEPGVEVIFYLSDREPLRLGSGEYTAEELCIRAAQACRISPLCHNLFALYDENTKLWYAPNRTITVDDKMSLRLHYRMRFYFTNWHGTNDNEQSVWRHSPKKQKNGYEKKKIPDATPLLDASSLEYLFAQGQYDLVKCLAPIRDPKTEQDGHDIENECLGMAVLAISHYAMMKKMQLPELPKDISYKRYIPETLNKSIRQRNLLTRMRINNVFKDFLKEFNNKTICDSSVSTHDLKVKYLATLETLTKHYGAEIFETSMLLISSENEMNWFHSNDGGNVLYYEVMVTGNLGIQWRHKPNVVSVEKEKNKLKRKKLENKHKKDEEKNKIREEWNNFSYFPEITHIVIKESVVSINKQDNKKMELKLSSHEEALSFVSLVDGYFRLTADAHHYLCTDVAPPLIVHNIQNGCHGPICTEYAINKLRQEGSEEGMYVLRWSCTDFDNILMTVTCFEKSEQVQGAQKQFKNFQIEVQKGRYSLHGSDRSFPSLGDLMSHLKKQILRTDNISFMLKRCCQPKPREISNLLVATKKAQEWQPVYPMSQLSFDRILKKDLVQGEHLGRGTRTHIYSGTLMDYKDDEGTSEEKKIKVILKVLDPSHRDISLAFFEAASMMRQVSHKHIVYLYGVCVRDVENIMVEEFVEGGPLDLFMHRKSDVLTTPWKFKVAKQLASALSYLEDKDLVHGNVCTKNLLLAREGIDSECGPFIKLSDPGIPITVLSRQECIERIPWIAPECVEDSKNLSVAADKWSFGTTLWEICYNGEIPLKDKTLIEKERFYESRCRPVTPSCKELADLMTRCMNYDPNQRPFFRAIMRDINKLEEQNPDIVSEKKPATEVDPTHFEKRFLKRIRDLGEGHFGKVELCRYDPEGDNTGEQVAVKSLKPESGGNHIADLKKEIEILRNLYHENIVKYKGICTEDGGNGIKLIMEFLPSGSLKEYLPKNKNKINLKQQLKYAVQICKGMDYLGSRQYVHRDLAARNVLVESEHQVKIGDFGLTKAIETDKEYYTVKDDRDSPVFWYAPECLMQSKFYIASDVWSFGVTLHELLTYCDSDSSPMALFLKMIGPTHGQMTVTRLVNTLKEGKRLPCPPNCPDEVYQLMRKCWEFQPSNRTSFQNLIEGFEALLK

SEQ ID NO: 294 - DNA, 5449 bp, Homo sapiens
cacacaggaaggagccgagtgggactttcctctcgctgcctcccggctctgcccgcccttcgaaagtccagggtccctgcccgctaggcaagttgcactcatggcacctccaagtgaagagacgcccctgatccctcagcgttcatgcagcctcttgtccacggaggctggtgccctgcatgtgctgctgcccgctcggggccccgggcccccccagcgcctatctttctcctttggggaccacttggctgaggacctgtgcgtgcaggctgccaaggccagcggcatcctgcctgtgtaccactccctctttgctctggccacggaggacctgtcctgctggttccccccgagccacatcttctccgtggaggatgccagcacccaagtcctgctgtacaggattcgcttttacttccccaattggtttgggctggagaagtgccaccgcttcgggctacgcaaggatttggccagtgctatccttgacctgccagtcctggagcacctctttgcccagcaccgcagtgacctggtgagtgggcgcctccccgtgggcctcagtctcaaggagcagggtgagtgtctcagcctggccgtgttggacctggcccggatggcgcgagagcaggcccagcggccgggagagctgctgaagactgtcagctacaaggcctgcctacccccaagcctgcgcgacctgatccagggcctgagcttcgtgacgcggaggcgtattcggaggacggtgcgcagagccctgcgccgcgtggccgcctgccaggcagaccggcactcgctcatggccaagtacatcatggacctggagcggctggatccagccggggccgccgagaccttccacgtgggcctccctggggcccttggtggccacgacgggctggggctgctccgcgtggctggtgacggcggcatcgcctggacccagggagaacaggaggtcctccagcccttctgcgactttccagaaatcgtagacattagcatcaagcaggccccgcgcgttggcccggccggagagcaccgcctggtcactgttaccaggacagacaaccagattttagaggccgagttcccagggctgcccgaggctctgtcgttcgtggcgctcgtggacggctacttccggctgaccacggactcccagcacttcttctgcaaggaggtggcaccgccgaggctgctggaggaagtggccgagcagtgccacggccccatcactctggactttgccatcaacaagctcaagactgggggctcacgtcctggctcctatgttctccgccgcagcccccaggactttgacagcttcctcctcactgtctgtgtccagaacccccttggtcctgattataagggctgcctcatccggcgcagccccacaggaaccttccttctggttggcctcagccgaccccacagcagtcttcgagagctcctggcaacctgctgggatggggggctgcacgtagatggggtggcagtgaccctcacttcctgctgtatccccagacccaaagaaaagtccaacctgatcgtggtccagagaggtcacagcccacccacatcatccttggttcagccccaatcccaataccagctgagtcagatgacatttcacaagatccctgctgacagcctggagtggcatgagaacctgggccatgggtccttcaccaagatttaccggggctgtcgccatgaggtggtggatggggaggcccgaaagacagaggtgctgctgaaggtcatggatgccaagcacaagaactgcatggagtcattcctggaagcagcgagcttgatgagccaagtgtcgtaccggcatctcgtgctgctccacggcgtgtgcatggctggagacagcaccatggtgcaggaatttgtacacctgggggccatagacatgtatctgcgaaaacgtggccacctggtgccagccagctggaagctgcaggtggtcaaacagctggcctacgccctcaactatctggaggacaaaggcctgccccatggcaatgtctctgcccggaaggtgctcctggctcgggagggggctgatgggagcccgcccttcatcaagctgagtgaccctggggtcagccccgctgtgttaagcctggagatgctcaccgacaggatcccctgggtggcccccgagtgtctccgggaggcgcagacacttagcttggaagctgacaagtggggcttcggcgccacggtctgggaagtgtttagtggcgtcaccatgcccatcagtgccctggatcctgctaagaaactccaattttatgaggaccggcagcagctgccggcccccaagtggacagagctggccctgctgattcaacagtgcatggcctatgagccggtccagaggccctccttccgagccgtcattcgtgacctcaatagcctcatctcttcagactatgagctcctctcagaccccacacctggtgccctggcacctcgtgatgggctgtggaatggtgcccagctctatgcctgccaagaccccacgatcttcgaggagagacacctcaagtacatctcacagctgggcaagggcaactttggcagcgtggagctgtgccgctatgacccgctaggcgacaatacaggtgccctggtggccgtgaaacagctgcagcacagcgggccagaccagcagagggactttcagcgggagattcagatcctcaaagcactgcacagtgatttcattgtcaagtatcgtggtgtcagctatggcccgggccgccagagcctgcggctggtcatggagtacctgcccagcggctgcttgcgcgacttcctgcagcggcaccgcgcgcgcctcgatgccagccgcctccttctctattcctcgcagatctgcaagggcatggagtacctgggctcccgccgctgcgtgcaccgcgacctggccgcccgaaacatcctcgtggagagcgaggcacacgtcaagatcgctgacttcggcctagctaagctgctgccgcttgacaaagactactacgtggtccgcgagccaggccagagccccattttctggtatgcccccgaatccctctcggacaacatcttctctcgccagtcagacgtctggagcttcggggtcgtcctgtacgagctcttcacctactgcgacaaaagctgcagcccctcggccgagttcctgcggatgatgggatgtgagcgggatgtccccgccctctgccgcctcttggaactgctggaggagggccagaggctgccggcgcctcctgcctgccctgctgaggttcacgagctcatgaagctgtgctgggcccctagcccacaggaccggccatcattcagcgccctgggcccccagctggacatgctgtggagcggaagccgggggtgtgagactcatgccttcactgctcacccagagggcaaacaccactccctgtccttttcatagctcctgcccgcagacctctggattaggtctctgttgactggctgtgtgaccttaggcccggagctgcccctctctgggcctcagaggccttatgagggtcctctacttcaggaacacccccatgacattgcatttgggggggctcccgtggcctgtagaatagcctgtggcctttgcaatttgttaaggttcaagacagatgggcatatgtgtcagtggggctctctgagtcctggcccaaagaagcaaggaaccaaatttaagactctcgcatcttcccaaccccttaagccctggccccctgagtttccttttctgtctctctctttttattttttttatttttatttttatttttgagacagagcctcgctctgttacccagggtggagtgcagtggtgcgatctcggctcagtgcaacctctgcttcccaggttcaagcgattctcctgcctcagcctcccgagtagctgggattacaggtgtgcaccaccacacccggctaattttttttatttttaatagagatgaggtttcaccatgatggccaggctgatctcgaactcctaacctcaagtgatcctcccacctcagcctcccaaagtgttggaataataggcatgagccactgcacccaggcttttttttttttaaatttattattattatttttaagagacaggatcttgctacgttgcccaggctggtcttgaactcctgggctacagtgatcctcctgccttatcctcctaaatagctgggactacagcacctagttttgagtttcctgtcttatttccaatggggacattcatgtagctttttttttttttttttttttgagacggagtctcgctctgtcgcccaggctggagtacagtggcgcaatctaggctcactgcaagctccgcctcctgggttcacaccattctctcgcctcagcctcccaagtagctgggactacaggcgcccgccaccacacccggctaattttttgtatttttagtagagacggggtttcaccttgttagccaggatggtttccatctcctgacctcgtgatctgcccgtctcggcctcccaaagtgctgggattacaggcatgagccactgcgcccggccctcatgtagctttaaatgtatgatctgacttctgctccccgatctctgtttctctggaggaagccaaggacaagagcagttgctgtggctgggactctgccttttaggggagcccgtgtatctctttgggatcctgaaagggggcaggaaaggctggggtcccagtccaccctaatggtatctgagtgtcctagggcttcagttttcccacctgtccaatgggaccctttctgtcctcaccctacaaggggcacaaagggatgacaccaaacctggcaggaacttttcacgcaatcaagggaaggaaaggcattcctggcagagggaacagcatgccaagcgtgagaaggctcagagtaaggaggttaagagcccaagtattggagcctacagttttgccccttccatgcagtgtgacagtgggcaagttcctttccctctctgggtctcagttctgtcccctgcaaaatggtcagagcttaccccttggctgtgcagggtcaactttctgactggtgagagggattctcatgcaggttaagcttctgctgctcctcctcacctgcaaagcttttctgccacttttgcctccttggaaaactcttatccatctctcaaaactccagctaccacatccttgcagccttccctcatatacccccactactactgtagccctgtccttccctccagccccactctggccctggggctggggaagtgtctgtgtccagctgtctcccctgacctcagggttccttgggggctgggctgaggcctcagtacagagggggctctggaaatgtttgttgactgaataaaggaattcagtggaaaaaaaaaaaaaaaaa

SEQ ID NO: 295 - protein, 1124 aa, Homo sapiens
MAPPSEETPLIPQRSCSLLSTEAGALHVLLPARGPGPPQRLSFSFGDHLAEDLCVQAAKASGILPVYHSLFALATEDLSCWFPPSHIFSVEDASTQVLLYRIRFYFPNWFGLEKCHRFGLRKDLASAILDLPVLEHLFAQHRSDLVSGRLPVGLSLKEQGECLSLAVLDLARMAREQAQRPGELLKTVSYKACLPPSLRDLIQGLSFVTRRRIRRTVRRALRRVAACQADRHSLMAKYIMDLERLDPAGAAETFHVGLPGALGGHDGLGLLRVAGDGGIAWTQGEQEVLQPFCDFPEIVDISIKQAPRVGPAGEHRLVTVTRTDNQILEAEFPGLPEALSFVALVDGYFRLTTDSQHFFCKEVAPPRLLEEVAEQCHGPITLDFAINKLKTGGSRPGSYVLRRSPQDFDSFLLTVCVQNPLGPDYKGCLIRRSPTGTFLLVGLSRPHSSLRELLATCWDGGLHVDGVAVTLTSCCIPRPKEKSNLIVVQRGHSPPTSSLVQPQSQYQLSQMTFHKIPADSLEWHENLGHGSFTKIYRGCRHEVVDGEARKTEVLLKVMDAKHKNCMESFLEAASLMSQVSYRHLVLLHGVCMAGDSTMVQEFVHLGAIDMYLRKRGHLVPASWKLQVVKQLAYALNYLEDKGLPHGNVSARKVLLAREGADGSPPFIKLSDPGVSPAVLSLEMLTDRIPWVAPECLREAQTLSLEADKWGFGATVWEVFSGVTMPISALDPAKKLQFYEDRQQLPAPKWTELALLIQQCMAYEPVQRPSFRAVIRDLNSLISSDYELLSDPTPGALAPRDGLWNGAQLYACQDPTIFEERHLKYISQLGKGNFGSVELCRYDPLGDNTGALVAVKQLQHSGPDQQRDFQREIQILKALHSDFIVKYRGVSYGPGRQSLRLVMEYLPSGCLRDFLQRHRARLDASRLLLYSSQICKGMEYLGSRRCVHRDLAARNILVESEAHVKIADFGLAKLLPLDKDYYVVREPGQSPIFWYAPESLSDNIFSRQSDVWSFGVVLYELFTYCDKSCSPSAEFLRMMGCERDVPALCRLLELLEEGQRLPAPPACPAEVHELMKLCWAPSPQDRPSFSALGPQLDMLWSGSRGCETHAFTAHPEGKHHSLSFS

SEQ ID NO: 296 - DNA, 5171 bp, Homo sapiens
ggcgggaggagagtcggcggccggagccgtcaccccgggcggggacccagcgcaggcaactccgcgcggcggcccggccgagggagggagcgagcgggcgggcgggcaagccagacagctgggccggagcagccgcgggcgcccgaggggccgagcgagattgtaaaccatggctgtgtggatacaagctcagcagctccaaggagaagcccttcatcagatgcaagcgttatatggccagcattttcccattgaggtgcggcattatttatcccagtggattgaaagccaagcatgggactcagtagatcttgataatccacaggagaacattaaggccacccagctcctggagggcctggtgcaggagctgcagaagaaggcagagcaccaggtgggggaagatgggtttttactgaagatcaagctggggcactatgccacacagctccagaacacgtatgaccgctgccccatggagctggtccgctgcatccgccatatattgtacaatgaacagaggttggtccgagaagccaacaatggtagctctccagctggaagccttgctgatgccatgtcccagaaacacctccagatcaaccagacgtttgaggagctgcgactggtcacgcaggacacagagaatgagttaaaaaagctgcagcagactcaggagtacttcatcatccagtaccaggagagcctgaggatccaagctcagtttggcccgctggcccagctgagcccccaggagcgtctgagccgggagacggccctccagcagaagcaggtgtctctggaggcctggttgcagcgtgaggcacagacactgcagcagtaccgcgtggagctggccgagaagcaccagaagaccctgcagctgctgcggaagcagcagaccatcatcctggatgacgagctgatccagtggaagcggcggcagcagctggccgggaacggcgggccccccgagggcagcctggacgtgctacagtcctggtgtgagaagttggccgagatcatctggcagaaccggcagcagatccgcagggctgagcacctctgccagcagctgcccatccccggcccagtggaggagatgctggccgaggtcaacgccaccatcacggacattatctcagccctggtgaccagcacgttcatcattgagaagcagcctcctcaggtcctgaagacccagaccaagtttgcagccactgtgcgcctgctggtgggcgggaagctgaacgtgcacatgaaccccccccaggtgaaggccaccatcatcagtgagcagcaggccaagtctctgctcaagaacgagaacacccgcaatgattacagtggcgagatcttgaacaactgctgcgtcatggagtaccaccaagccacaggcacccttagtgcccacttcaggaatatgtccctgaaacgaattaagaggtcagaccgtcgtggggcagagtcggtgacagaagaaaaatttacaatcctgtttgaatcccagttcagtgttggtggaaatgagctggtttttcaagtcaagaccctgtccctgccagtggtggtgatcgttcatggcagccaggacaacaatgcgacggccactgttctctgggacaatgcttttgcagagcctggcagggtgccatttgccgtgcctgacaaagtgctgtggccacagctgtgtgaggcgctcaacatgaaattcaaggccgaagtgcagagcaaccggggcctgaccaaggagaacctcgtgttcctggcgcagaaactgttcaacaacagcagcagccacctggaggactacagtggcctgtctgtgtcctggtcccagttcaacagggagaatttaccaggacggaattacactttctggcaatggtttgacggtgtgatggaagtgttaaaaaaacatctcaagcctcattggaatgatggggccattttggggtttgtaaacaagcaacaggcccatgacctactcattaacaagccagatgggaccttcctcctgagattcagtgactcagaaattggcggcatcaccattgcttggaagtttgattctcaggaaagaatgttttggaatctgatgccttttaccaccagagacttctccattcggtccctagccgaccgcttgggagacttgaattaccttatctacgtgtttcctgatcggccaaaagatgaagtatactccaaatactacacaccagttccctgcgagtctgctactgctaaagctgttgatggatacgtgaagccacagatcaagcaagtggtccctgagtttgtgaacgcatctgcagatgccgggggcggcagcgccacgtacatggaccaggccccctccccagctgtgtgtccccaggctcactataacatgtacccacagaaccctgactcagtccttgacaccgatggggacttcgatctggaggacacaatggacgtagcgcggcgtgtggaggagctcctgggccggccaatggacagtcagtggatcccgcacgcacaatcgtgaccccgcgacctctccatcttcagcttcttcatcttcaccagaggaatcactcttgtggatgttttaattccatgaatcgcttctcttttgaaacaatactcataatgtgaagtgttaatactagttgtgaccttagtgtttctgtgcatggtggcaccagcgaagggagtgcgagtatgtgtttgtgtgtgtgtgtgtgtgtgtgtgtgtgtgcgtgtttgcacgttatggtgtttctccctctcactgtctgagagtttagttgtagcagaggggccacagacagaagctgtggtggtttttactttgtgcaaaaaggcagtgagtttcgtgaagcctggaagttggccatgtgtcttaagagtggctggactttgacatgtggctgtttgaataagagaaggacaaagggaggagaaagcacatgtgctccagtgagtcttcgtcactctgtctgccaagcaattgatatataaccgtgattgtctctgcttttcttctgaaatgtagataactgctttttgacaaagagagccttccctctcccccacccctgtgttcttgggtaggaatgggaaaaggggcaacctacaaagattgttggggcaagggaagtcacaagctttcggatgggcggtggcttttcacaaaacatttagctcatcttattctctctttgtcctctctcccctcctgcccgcccgcaccctggaattgccactcagttcctctgggtgtgcacatatgtttggagaaatagaggagagaaaagagggccacgtaactgagagcttacagtgccaatgccgtttgtgttctggccagagtggagtgcgcagccctgactcccaggcgctgagattgttgcctggttacccaggaagctgctgttccggctgcccagcctttctctgagccagcggatgcacagtccgtggccttcttcaggcttattgatgatgctttttgcaaatgttgaatcatggttctgtttctaagttggatcttttttgttttctccttgccaccctaatttgacatcaaaattctctcttgtgcattgggccctgggtcattcaaacccaggtcacctcattccccttctctgttcacacctaatgtcttgaagagtaggtagcagcagtgtgggctgaacctaggccagcttgcttagcgggtcaccctgctgtgaagtcctggcaggtgttggtaatgtgtggaaatgcagtcagcaagtttgctggggagtttgataaaagtataaaacaaaacaaaaaaagcctcggtataattttgttccacgacttcttctgtagctttacaccagaaggaaggaatgggctacagcaggtagtggaggaagaggggggtgagcaggtgtattaaaatagcttacgggtaaggcctaaaaggtcacccctcggccccctctccaaaagaagggcatgggcacccccaggagaggatggccccaaaaaccttatttttatacatgagagtaaataaacatattttttttacaaaaataacttctgaatttatcagtgttttgccgttaaaaatattcctctatagtaaattatttattggaagatgacttttttaaagctgccgtttgccttggcttggtttcatacactgatttatttttctatgccaggcagtagagtctctctgcctctgaggagcaggctacccgcatcccactcagcccctccctacccctcaagatttgatgaaaattccaaccatgaggatgggtgcatcggggaagggtgagaaggagagcctgcctgctcagggatccaggctcgtagagtcactccctgcccgtctcccagagatgcttcaccagcacctgcctctgagacctcgctctctgttccagcaaccctggttggggggtcagacttgatacactttcaggttgggagtggacccaccccagggcctgctgaggacagagcagccaggccgtcctggctcactttgcagttggcactgggttggggaggaagagagctgatgagtgtggcttccctgagctggggtttccctgcttgtccagttgtgagctgtcctcggtgttaccgaggctgtgcctagagagtggagatttttgatgaaaggtgtgctcgctctctgcgttctatcttctctctcctccttgttcctgcaaaccacaagataaaggtagtggtgtgtctcgaccccatcagcctctcacccactcccagacacacacaagtcctcaaaagtttcagctccgtgtgtgagatgtgcaggttttttctagggggtagggggagactaaaatcgaatataacttaaaatgaaagtatactttttataatttttctttttaaaacttggtgaaattatttcagatacatattttagtgtcaaggcagattagttatttagccaccaaaaaaaagtattgtgtacaatttggggcctcaaatttgactctgcctcaaaaaaaagaaatatatcctatgcagagttacagtcacaaagttgtgtattttatgttacaataaagccttcctctgaagggaaaaaaaaaaaaaaaaaaaaaaaaaaaaaaaaaaaaaaaaaaaaaaaaaaaaaaaaaaaaaaaaaaaaaaaaaaaaaa

SEQ ID NO: 297 - protein, 787 aa, Homo sapiens
MAVWIQAQQLQGEALHQMQALYGQHFPIEVRHYLSQWIESQAWDSVDLDNPQENIKATQLLEGLVQELQKKAEHQVGEDGFLLKIKLGHYATQLQNTYDRCPMELVRCIRHILYNEQRLVREANNGSSPAGSLADAMSQKHLQINQTFEELRLVTQDTENELKKLQQTQEYFIIQYQESLRIQAQFGPLAQLSPQERLSRETALQQKQVSLEAWLQREAQTLQQYRVELAEKHQKTLQLLRKQQTIILDDELIQWKRRQQLAGNGGPPEGSLDVLQSWCEKLAEIIWQNRQQIRRAEHLCQQLPIPGPVEEMLAEVNATITDIISALVTSTFIIEKQPPQVLKTQTKFAATVRLLVGGKLNVHMNPPQVKATIISEQQAKSLLKNENTRNDYSGEILNNCCVMEYHQATGTLSAHFRNMSLKRIKRSDRRGAESVTEEKFTILFESQFSVGGNELVFQVKTLSLPVVVIVHGSQDNNATATVLWDNAFAEPGRVPFAVPDKVLWPQLCEALNMKFKAEVQSNRGLTKENLVFLAQKLFNNSSSHLEDYSGLSVSWSQFNRENLPGRNYTFWQWFDGVMEVLKKHLKPHWNDGAILGFVNKQQAHDLLINKPDGTFLLRFSDSEIGGITIAWKFDSQERMFWNLMPFTTRDFSIRSLADRLGDLNYLIYVFPDRPKDEVYSKYYTPVPCESATAKAVDGYVKPQIKQVVPEFVNASADAGGGSATYMDQAPSPAVCPQAHYNMYPQNPDSVLDTDGDFDLEDTMDVARRVEELLGRPMDSQWIPHAQS

SEQ ID NO: 298 - DNA, 1560 bp, Homo sapiens
agaggaaacgtgtgggtggggaggggtagtgggtgagggacccaggttcctgacacagacagactacacccagggaatgaagagcaagcgccatgttgaagccatcattaccattcacatccctcttattcctgcagctgcccctgctgggagtggggctgaacacgacaattctgacgcccaatgggaatgaagacaccacagctgatttcttcctgaccactatgcccactgactccctcagtgtttccactctgcccctcccagaggttcagtgttttgtgttcaatgtcgagtacatgaattgcacttggaacagcagctctgagccccagcctaccaacctcactctgcattattggtacaagaactcggataatgataaagtccagaagtgcagccactatctattctctgaagaaatcacttctggctgtcagttgcaaaaaaaggagatccacctctaccaaacatttgttgttcagctccaggacccacgggaacccaggagacaggccacacagatgctaaaactgcagaatctggtgatcccctgggctccagagaacctaacacttcacaaactgagtgaatcccagctagaactgaactggaacaacagattcttgaaccactgtttggagcacttggtgcagtaccggactgactgggaccacagctggactgaacaatcagtggattatagacataagttctccttgcctagtgtggatgggcagaaacgctacacgtttcgtgttcggagccgctttaacccactctgtggaagtgctcagcattggagtgaatggagccacccaatccactgggggagcaatacttcaaaagagaatcctttcctgtttgcattggaagccgtggttatctctgttggctccatgggattgattatcagccttctctgtgtgtatttctggctggaacggacgatgccccgaattcccaccctgaagaacctagaggatcttgttactgaataccacgggaacttttcggcctggagtggtgtgtctaagggactggctgagagtctgcagccagactacagtgaacgactctgcctcgtcagtgagattcccccaaaaggaggggcccttggggaggggcctggggcctccccatgcaaccagcatagcccctactgggcccccccatgttacaccctaaagcctgaaacctgaaccccaatcctctgacagaagaaccccagggtcctgtagccctaagtggtactaactttccttcattcaacccacctgcgtctcatactcacctcaccccactgtggctgatttggaattttgtgcccccatgtaagcaccccttcatttggcattccccacttgagaattacccttttgccccgaacatgtttttcttctccctcagtctggcccttccttttcgcaggattcttcctccctccctctttccctcccttcctctttccatctaccctccgattgttcctgaaccgatgagaaataaagtttctgttgataatcatcaaaaaaaaaaaaaaaaaaaaaaaaaaaaaaa

SEQ ID NO: 299 - protein, 369 aa, Homo sapiens
MLKPSLPFTSLLFLQLPLLGVGLNTTILTPNGNEDTTADFFLTTMPTDSLSVSTLPLPEVQCFVFNVEYMNCTWNSSSEPQPTNLTLHYWYKNSDNDKVQKCSHYLFSEEITSGCQLQKKEIHLYQTFVVQLQDPREPRRQATQMLKLQNLVIPWAPENLTLHKLSESQLELNWNNRFLNHCLEHLVQYRTDWDHSWTEQSVDYRHKFSLPSVDGQKRYTFRVRSRFNPLCGSAQHWSEWSHPIHWGSNTSKENPFLFALEAVVISVGSMGLIISLLCVYFWLERTMPRIPTLKNLEDLVTEYHGNFSAWSGVSKGLAESLQPDYSERLCLVSEIPPKGGALGEGPGASPCNQHSPYWAPPCYTLKPET

(19) United States
(12) Patent Application Publication
(10) Pub. No.: US 2015/0050274 A1
(43) Pub. Date: Feb. 19, 2015

(54) METHODS AND BIOMARKERS FOR DETECTION AND TREATMENT OF MATURE T-CELL LEUKEMIA

(71) Applicant: The Regents of the University of Michigan, Ann Arbor, MI (US)

(72) Inventors: Kojo Elenitoba-Johnson, Ann Arbor, MI (US); Mark J. Kiel, Ann Arbor, MI (US); Thirunavukkarasu Velusamy, Ann Arbor, MI (US); Anagh Sahasrabuddhe, Ann Arbor, MI (US); Delphine Rolland, Ann Arbor, MI (US); Megan Lim, Ann Arbor, MI (US)

(21) Appl. No.: 14/459,928

(22) Filed: Aug. 14, 2014

Related U.S. Application Data
(60) Provisional application No. 61/866,297, filed on Aug. 15, 2013.

(51) Int. Cl. C12Q 1/68; G01N 33/573

(52) U.S. Cl. CPC C12Q 1/6886; G01N 33/573; C12Q 2600/118; G01N 2333/91205; G01N 2800/52; C12Q 2600/156
USPC 424/133.1; 506/2; 514/265.1; 435/7.4; 514/210.21; 514/235.8; 514/211.08

(57) ABSTRACT

The present invention relates to methods and biomarkers for detection and characterization of mature T-cell neoplasias/leukemias (e.g., T-cell prolymphocytic leukemia, Sezary syndrome) in biological samples (e.g., tissue samples, blood samples, plasma samples, cell samples, serum samples).

### CROSS-REFERENCE TO RELATED APPLICATIONS

The present application claims priority to pending U.S. Provisional Patent Application No. 61/866,297, filed Aug. 15, 2013, the contents of which is incorporated by reference in its entirety.

### STATEMENT REGARDING FEDERALLY SPONSORED RESEARCH OR DEVELOPMENT

This invention was made with government support under CA136905 and CA140806 awarded by the National Institutes of Health. The Government has certain rights in the invention.

### FIELD OF THE INVENTION

The present invention relates to methods and biomarkers for detection and characterization of mature T-cell neoplasias/leukemias (e.g., T-cell prolymphocytic leukemia, Sezary syndrome) in biological samples (e.g., tissue samples, blood samples, plasma samples, cell samples, serum samples).

### BACKGROUND OF THE INVENTION

The genetic alterations underlying the pathogenesis of mature (post-thymic) T-cell leukemias are largely unknown and treatment options are often ineffective and patient outcomes poor. Among the mature T-cell leukemias, T-cell prolymphocytic leukemia (T-PLL) is an aggressive neoplasm of mature T-lymphocytes characterized by a rapid clinical course, resistance to conventional chemotherapy and poor median survival (less than 7.5 months) (see, e.g., Matutes, E. et al. Blood 78, 3269-3274 (1991); herein incorporated by reference in its entirety). An additional T-cell leukemia, Sezary Syndrome (SS), is an aggressive mature T-cell leukemic disease with median 5-year survival of less than 20% (see, e.g., Willemze, R. et al. Blood 105, 3768-3785, doi:2004-09-3502 (2005); herein incorporated by reference in its entirety).

Additional insight into the pathogenesis of mature T-cell leukemias is needed. In addition, better, more effective non-invasive tests for early detection of mature T-cell leukemias are needed to lower the morbidity and mortality associated with such cancers.

### SUMMARY OF THE INVENTION

The present invention relates to methods and biomarkers for detection and characterization of mature T-cell neoplasias/leukemias (e.g., T-cell prolymphocytic leukemia, Sezary syndrome) in biological samples (e.g., tissue samples, blood samples, plasma samples, cell samples, serum samples).

In certain embodiments, the present invention provides methods for detecting one or more JAK/STAT pathway variants associated with a mature T-cell leukemia in a subject, comprising contacting a sample from a subject with a JAK/STAT pathway variant detection assay under conditions that the presence of a JAK/STAT pathway variant associated with a mature T-cell leukemia is determined; and diagnosing the subject with a mature T-cell leukemia when one or more of the JAK/STAT pathway variants are present in the sample.

Similarly, in certain embodiments, the present invention provides uses of a variant JAK/STAT pathway nucleic acid or polypeptide for detecting a mature T-cell leukemia in a subject.

In certain embodiments, the present invention further provides methods for determining a decreased time to adverse outcome in a subject diagnosed with a mature T-cell leukemia, comprising contacting a sample from a subject with a JAK/STAT pathway variant detection assay under conditions that the presence of a JAK/STAT pathway variant associated with a mature T-cell leukemia is determined; and detecting a decreased time to adverse outcome in the subject when the JAK/STAT pathway variants are present in the sample. In some embodiments, the adverse outcome is selected from the group consisting of relapse of the mature T-cell leukemia, metastasis, or death.

In some embodiments for such methods and uses, the subject is a human (e.g., a human subject being screened for a mature T-cell leukemia) (e.g., a human subject at risk for developing a mature T-cell leukemia) (e.g., a human subject assessing the effectiveness of a mature T-cell leukemia treatment regimen).

In some embodiments, the biological sample is selected from the group consisting of a tissue sample, a cell sample, and a blood sample.

In some embodiments, the one or more JAK/STAT pathway variants encodes a loss of function mutation and/or a gain of function mutation.

In some embodiments, the JAK/STAT pathway variant is one or more variants selected from a JAK1 variant, a JAK3 variant, a STAT5B variant, and an IL2RG variant.

In some embodiments, the JAK1 variant is a JAK1 polypeptide having an amino acid sequence differing from a wild type JAK1 amino acid sequence. In some embodiments, the JAK1 variant is a JAK1 nucleic acid sequence encoding a JAK1 polypeptide having an amino acid sequence differing from a wild type JAK1 amino acid sequence. In some embodiments, the one or more JAK1 variants is a JAK1 polypeptide having, in comparison to wild type, an amino acid variation selected from the group consisting of JAK1 p.F636L, JAK1 p.G646C, JAK1 p. Y654F, JAK1 p.V658F, JAK1 p. S703I, and JAK1 p.T901R.

In some embodiments, the JAK3 variant is a JAK3 polypeptide having an amino acid sequence differing from a wild type JAK3 amino acid sequence. In some embodiments, the JAK3 variant is a JAK3 nucleic acid sequence encoding a JAK3 polypeptide having an amino acid sequence differing from a wild type JAK3 amino acid sequence. In some embodiments, the one or more JAK3 variants is a JAK3 polypeptide having, in comparison to wild type, an amino acid variation selected from the group consisting of JAK3 p.ΔKNC563, AK3 p.M511I, JAK3 p. A573V, JAK3 p.R657. JAK3 p.Y980, JAK3 p.G662W, JAK3 p.P664T, JAK3 p. Y981, and JAK3 p. S9891.

In some embodiments, the STAT5B variant is a STAT5B polypeptide having an amino acid sequence differing from a wild type STAT5B amino acid sequence. In some embodiments, the STAT5B variant is a STAT5B nucleic acid sequence encoding a STAT5B polypeptide having an amino acid sequence differing from a wild type STAT5B amino acid sequence. In some embodiments, the one or more STAT5B variants is a STAT5B polypeptide having, in comparison to wild type, an amino acid variation selected from the group consisting of STAT5B p.T628S, STAT5B p.N642H, STAT5B p.Y699, STAT5B p.R659c, STAT5B p.Q706L, and STAT5B p.Y665H.

In some embodiments, the IL2RG variant is a IL2RG polypeptide having an amino acid sequence differing from a wild type IL2RG amino acid sequence. In some embodiments, the IL2RG variant is a IL2RG nucleic acid sequence encoding a IL2RG polypeptide having an amino acid sequence differing from a wild type IL2RG amino acid sequence. In some embodiments, the one or more IL2RG variants is a IL2RG polypeptide having, in comparison to wild type, an amino acid variation selected from the group consisting of IL2RG p.Y325, IL2RG p.ΔGSM268, and IL2RG p. K315E.

In some embodiments, the mature T-cell leukemia is T-cell prolymphocytic leukemia and the one or more JAK/STAT pathway variants is JAK/STAT pathway polypeptide having, in comparison to wild type, an amino acid variation selected from the group consisting of JAK1 p.V658F, JAK1 p. S703I, JAK1 p.T901R, JAK3 p.ΔKNC563, JAK3 p.M511I, JAK3 p. A573V, JAK3 p.R657, STAT5B p.T628S, STAT5B p.R659c, STAT5B p.Q706L, STAT5B p.N642H, STAT5B p.Y665H, IL2RG p.ΔGSM268, and IL2RG p. K315E.

In some embodiments, the mature T-cell leukemia is Sezary syndrome and the one or more JAK/STAT pathway variants is JAK/STAT pathway polypeptide having, in comparison to wild type, an amino acid variation selected from the group consisting of JAK1 p.Y654F, JAK3 p.A573V, JAK3 p.Y980, JAK3 p. Y981, JAK3 p. S9891, STAT5B p.Y699, STAT5B p.N642H, and I12RG p.Y325.

In some embodiments, the determining comprises detecting variant JAK1, JAK3, STAT5B, and IL2RG nucleic acids or polypeptides.

In some embodiments, the detecting variant JAK1, JAK3, STAT5B, and IL2RG nucleic acids comprises one or more nucleic acid detection method selected from the group consisting of sequencing, amplification and hybridization.

In some embodiments, the determining comprises a computer implemented method. In some embodiments, the computer implemented method comprises analyzing JAK1, JAK3, STAT5B, and IL2RG variant information and displaying the information to a user.

In some embodiments, the methods and uses further comprise the step of treating the subject for a mature T-cell leukemia and monitoring the subject for the presence of JAK1, JAK3, STAT5B, and IL2RG variants associated with the mature T-cell leukemia. For example, in some embodiments, the methods and uses further comprise the step of treating the subject for a mature T-cell leukemia under condition such that at least one symptom of the mature T-cell leukemia is diminished or eliminated.

In some embodiments, the treating comprises inhibiting JAK1, JAK3, STAT5B, and/or IL2RG expression and/or activity. In some embodiments, inhibiting STAT5B expression and/or activity is accomplished through administration of an agent configured to inhibit STAT5B expression (e.g., pimozide). In some embodiments, inhibiting JAK1 and/or JAK3 expression and/or activity is accomplished through administration of an agent configured to inhibit JAK1 and/or JAK3 expression and/or activity (e.g., ruxolitinib, tofacitinib, baricitinib, CYT387, and/or lestaurtinib).

In some embodiments, the methods further comprise administering one or more agents for treating a mature T-cell leukemia. In some embodiments, the one or more agents is selected from the group consisting of a purine analog (e.g., pentostatin, fludarabine, cladrbine), chlorambucil, cyclophosphamide, doxorubicin, vincristine, prednisone (CHOP), cyclophosphamide, vincristine, prednisone (COP), and vincristine, doxorubicin, prednisone, etoposide, cyclophosphamide, bleomycin, alemtuzumab, and vorinostat.

In some embodiments, the methods and uses further comprise the step of detecting a variant in one or more additional genes and/or polypeptides associated with a mature T-cell leukemia. In some embodiments, the one or more genes or polypeptides are selected from the group consisting of CHEK2, EZH2, and FBXW10.

Additional embodiments will be apparent to persons skilled in the relevant art based on the teachings contained herein.

### DESCRIPTION OF THE DRAWINGS

FIG. 1 (gray-scaled) shows a phosphoproteomic screen of mature T-cell leukemia cell line HUT78. a, Schematic of phosphoproteomic screening analysis. Further details of experimental technique can be found in the Supplemental Information. b, Tyrosine-phosphorylated proteins identified by mass spectrometry in HUT78 T-cell leukemia cell line; pY indicates phosphorylated residue, boxed entries highlight JAK3 and STAT5B peptides.

FIG. 2 (gray-scaled) shows dual phosphoproteomic and genome sequencing screen identifies JAK-STAT activation in the mature T-cell leukemia cell line HUT78. a-c, Tandem mass-spectra confirming tyrosine phosphorylation of IL2RG p.Y325, JAK3 p.Y980/Y981 and STAT5B p.Y699 residues in HUT78 cells. d, Summary of results of phosphotyrosine proteomic screening of HUT78 cells (left) and summary of novel mutations in kinases and their targets identified by WES (right) highlighting the presence of altered JAK-STAT pathway in both datasets. e-g, JAK1 (p.Y654F) and JAK3 (p.A573V) mutations in HUT78 cells discovered through WES and confirmed by Sanger sequencing. HUT78 cells are haploinsufficient at the JAK3 locus leading to monoallelic amplification.

FIG. 3 (gray-scaled) shows representative diagnostic material for T-PLL samples. a, Cytology of representative T-PLL case. b, TCR locus florescence in situ hybridization (FISH) analysis demonstrating typical inv(14) in a representative T-PLL sample.

FIG. 4 (gray-scaled) shows structural alterations involving TCL1B/MTCP locus in index T-PLL samples. Ideogram of structural alterations in 4 index T-PLL cases subjected to genomic sequencing involving chr14 (left) and/or chrX (right); the smaller regions (non-gray scaled “red”) and the 22,976,660, 22,974,099, 22,962,913, 96,175,046, 96,082,911, 96,153,448 values represents the intrachromosomal translocation, inv(14); the second region for the chr14 (non-gray scaled “blue”) and the values 22,946,702 and 154,299,773 indicates the interchromosomal translocation, t(14;X); breakpoint positions for each translocation are shown.

FIG. 5 (gray-scaled) shows activating IL2R-JAK1/3-STAT5B axis mutations in T-cell leukemia. a-d, Representative IL2RG, JAK1, JAK3 and STAT5B mutations identified in primary T-PLL cells by WGS/WES and confirmed somatic acquisition by Sanger sequencing of tumor (upper panels) and paired normal tissue (lower panels). Schematic representation of mutations in IL2RG (e), JAK1 (f), JAK3 (g) and STAT5B (h and 1) identified through WGS, WES or targeted Sanger sequencing of HUT78 cells (diamond) and primary T-cell leukemias T-PLL (circles) and SS (squares) and the cutaneous T-cell lymphoma MF (triangles). Mutations with confirmed somatic acquisition are shown as filled symbols, with mutations at residues otherwise previously identified in hematopoietic malignancy shown as grey symbols; variants where adequate matched constitutional DNA was not available are shown as open symbols. For T-PLL and MF, the mutations are concentrated in the pseudo-kinase domains (f and g, purple; JAK1 and JAK3) or the SH2 domain (light blue; STAT5B). Two additional variants were detected in the kinase domains of JAK1 and JAK3 (red); a single case of T-PLL harbored a somatic three amino acid deletion in the transmembrane domain of IL2RG (e, purple) as well as a somatic missense mutation in the cytoplasmic domain. A indicates small deletion of several contiguous amino acid residues. i, Frequency of JAK-STAT pathway mutations in mature T-cell leukemia/lymphoma. j, SH2 domain of the STAT5A protein (lylu) highlighting analogous residues of the STAT5B mutations p.N642H (gray-scaled blue), p.Y665H (gray-scaled purple) and p.T628S (gray-scaled red) demonstrating close 3-dimensional proximity of mutated STAT5B residues based on STAT5A crystal structure. k, Pseudokinase domain of JAK2 (4fvp) highlighting V617 (gray-scaled light blue) and analogous residues for the JAK3 mutations p.A573V (gray-scaled red), p.M511I (gray-scaled dark blue), p.ΔKNC563 (gray-scaled purple).

FIG. 6 (gray-scaled) shows copy-number variations in T-PLL. Circos diagram depicting aCGH data for 43 individual T-PLL samples (inner data tracks) and a histogram representation of all samples to show areas of recurrent gain or loss of chromosomal material (outer data tracks; gray-scaled blue represents loss, gray-scaled red indicates gain); arrow indicates recurrent loss of ATM locus on 11q; arrowhead represents alterations of chromosome 8 present in a majority of T-PLL genomes. Histogram data is presented as the number of cases with -logR values less than −0.2 (loss, blue) or greater than 0.2 (gain, red).

FIG. 7 (gray-scaled) shows distribution of ATM mutations in T-PLL. a, Protein diagram depicting all mutations in ATM identified by genome and exome sequencing of T-PLL samples. b-d, Representative Sanger sequencing electropherograms confirming the existence of the mutation in tumor samples (upper panels) and the absence of mutations in paired normal samples (lower panels).

FIG. 8 (gray-scaled) shows targeted inhibition of activated STAT5B signaling in primary T-cell leukemias. a-b, Mutated IL2RG (p.ΔGMS628), JAK1 (p.S703I), JAK3 (p.Q507P) and STAT5B (p.T628S and p.N642H) leads to increased activation of STAT5B transcriptional activity (a, bar graph; n=3 for each mutant in separate experiments; asterisk indicates p<0.05; dagger indicates p<0.001) and increased phosphorylation of STAT5B (b, Western blot; arrowhead indicates exogenous STAT5B, arrow indicates endogenous STAT5B) in HeLa cells. c, Enhanced cell proliferation in the presence of mutant p.T628S STAT5B protein in Ba/F3 cells (n=6; asterisk indicates p<0.01). d, Enhanced colony forming capacity in Jurkat T-cells (n=3; asterisk indicates p<0.01). e, Upregulation of pSTAT5B in representative primary T-PLL samples demonstrated by immunocytochemistry. Targeted inhibition of pSTAT5 with the small molecule inhibitor Pimozide leads to causes reduced proliferation (f), decreased viability (g) and diminished pSTAT5 levels (h) in primary T-PLL samples (n=3 independent replicates for f and g, asterisk indicates p<0.005; representative data is shown for T-PLL 25 in h; arrowhead in h indicates cleaved PARP associated with apoptotic activation). i, Pathway diagram illustrating the interaction of IL2RG, JAK1, JAK3 and STAT5B during IL2 cytokine activation. Cytokine binding to the extracellular portion of membrane-associated interleukin receptors induces conformational changes in the intracellular portion. Associated JAK non-receptor tyrosine kinases then auto-phosphorylate leading to STAT recruitment and activation through tyrosine phosphorylation. Activated STAT proteins then dimerize and translocate to the nucleus to regulate transcription of numerous genes involved in differentiation, proliferation and survival. Pimozide treatment inhibits STAT5 phosphorylation delimiting downstream transcriptional activity of activating mutations in cytokine receptor-JAK-STAT proteins (highlighted green).

FIG. 9 (gray-scaled) shows read alignment of WGS data supporting JAK1 mutations in T-PLL index cases. The total individual reads supporting variant calling of the p.V658F (a) and p.S703I (b) mutations in index T-PLL samples is shown. Nucleotides with deviation from reference sequence are highlighted. The mutations as well as a synonymous single nucleotide polymorphism are boxed.

FIG. 10 (gray-scaled) shows three-dimensional apposition of selected residues affected by JAK1, JAK3 and STAT5B mutation in T-PLL. Amino acid sequence alignment of STAT5B (top) versus STAT5A (a, bottom), JAK2 (top) versus JAK1 (b, bottom) and JAK3 (c, bottom) in the region of recurrent mutations in T-PLL. Colored fill indicates identical amino acid; white, minus indicates disparate residues; white with colored text, + indicates similar residues); selected mutated residues are indicated in red.

FIGS. 11A and 11B provide the homo sapiens wild type nucleic acid sequence and amino acid sequence for JAK1, respectively.

FIGS. 11C and 11D provide the homo sapiens wild type nucleic acid sequence and amino acid sequence for JAK3, respectively.

FIGS. 11E and 11F provide the homo sapiens wild type nucleic acid sequence and amino acid sequence for STAT5B, respectively.

FIGS. 11G and 11H provide the homo sapiens wild type nucleic acid sequence and amino acid sequence for IL2RG is provided at FIGS. 11G and 11H, respectively.

FIG. 12 (gray-scaled) shows selected recurrently mutated genes in T-PLL by WES and WGS.

FIG. 13 shows JAK-STAT mutational status associated with patient demographic and clinical diagnostic and prognostic information. Index cases subjected to WGS are listed first. Deletions or point mutations in 11q23 locus or ATM gene, respectively are indicated. Karyotypic or immunohistochemical evidence of rearrangements involving the TCL1A/B or MTCP1 loci or TCL1 protein are indicated. Mutations identified in T-PLL cases are shown by gene. Cases for which no mutation was identified are indicated according to which method of mutation detection was employed. The patient's treatment status at time of specimen collection is also indicated as are the times from disease diagnosis to relapse or death and the patients' survival status.

### DEFINITIONS

To facilitate an understanding of the present invention, a number of terms and phrases are defined below:

As used herein, the term “mature T-cell leukemia” or “mature T-cell neoplasia” refers to a type of lymphoid leukemia that which affects mature T-cells. Examples of mature T-cell leukemia include, but are not limited to, T-cell prolymphocytic leukemia (T-PLL) and Sezary syndrome (SS). T-cell prolymphocytic leukemia (T-PLL) is a mature T-cell leukemia with aggressive behavior and predilection for blood, bone marrow, lymph nodes, liver, spleen, and skin involvement. SS is a type of cutaneous lymphoma wherein the affected cells are T-cells that have pathological quantities of mucopolysaccharides. As described herein, the terms “mature T-cell leukemia” and “mature T-cell neoplasia” are interchagable.

The term “JAK/STAT signaling pathway” or “JAK/STAT pathway” refers to a signaling pathway for transmitting information from chemical signals outside a cell, through the cell membrane, and into gene promoters on DNA in the cell nucleus, which causes DNA transcription and activity in the cell. The JAK-STAT system consists of three main components: (1) a receptor (2) Janus kinase (JAK) and (3) Signal Transducer and Activator of Transcription (STAT). Examples of JAK include JAK1, JAK2, JAK3, and TYK2. Examples of STAT include STAT1, STAT3, STAT5A, STAT5B, and STATE. In some embodiments, the cytokine that bind the receptor is IL2RG.

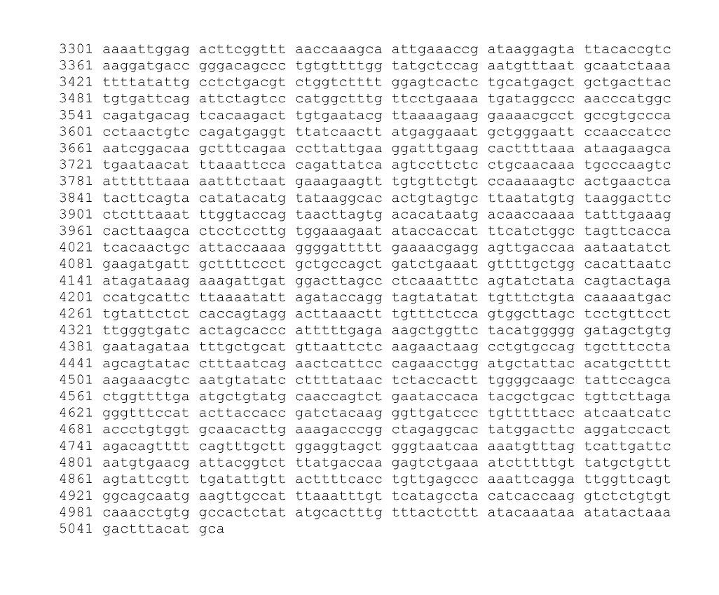

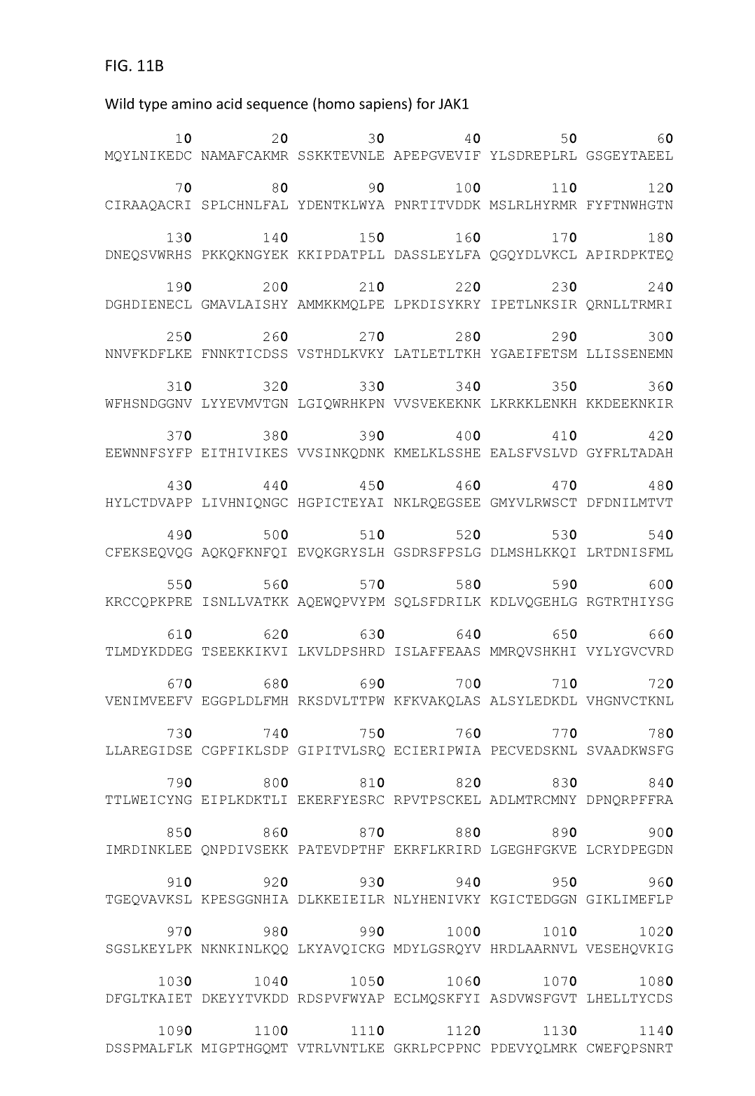

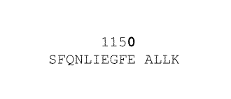

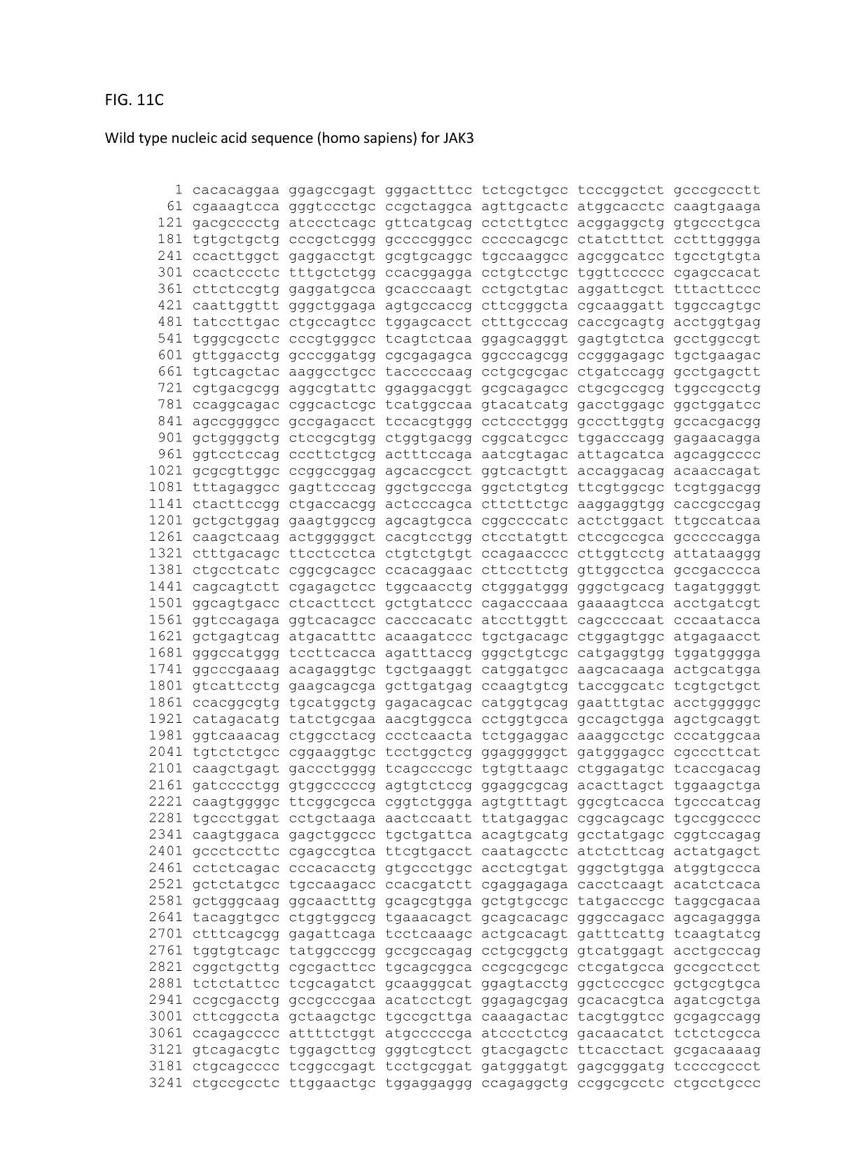

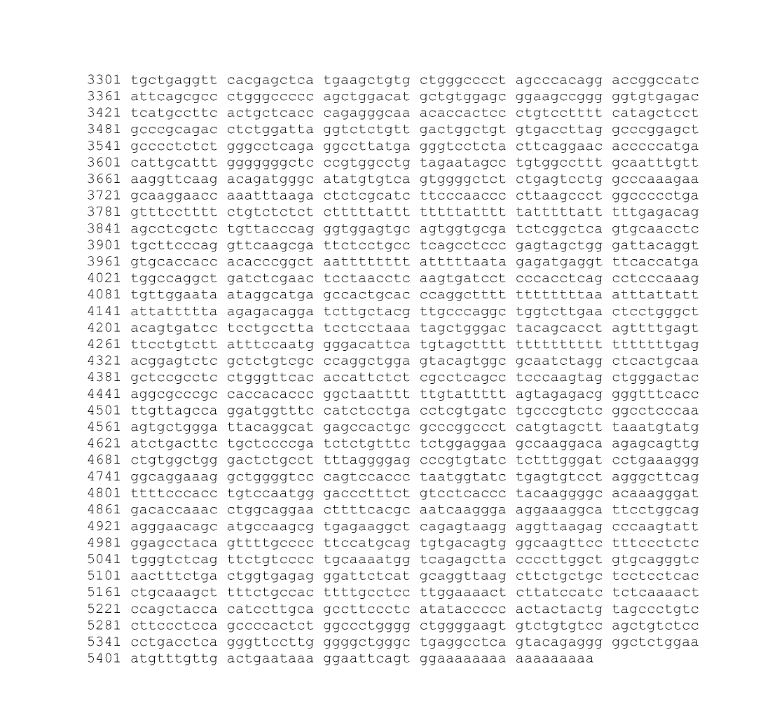

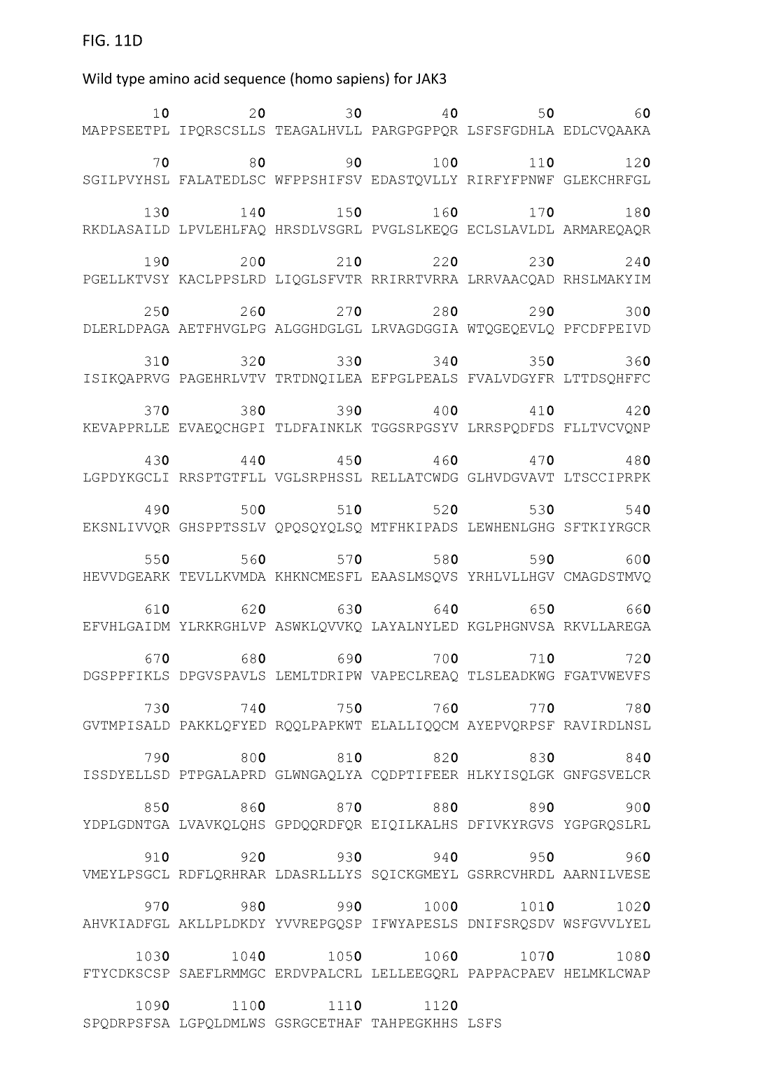

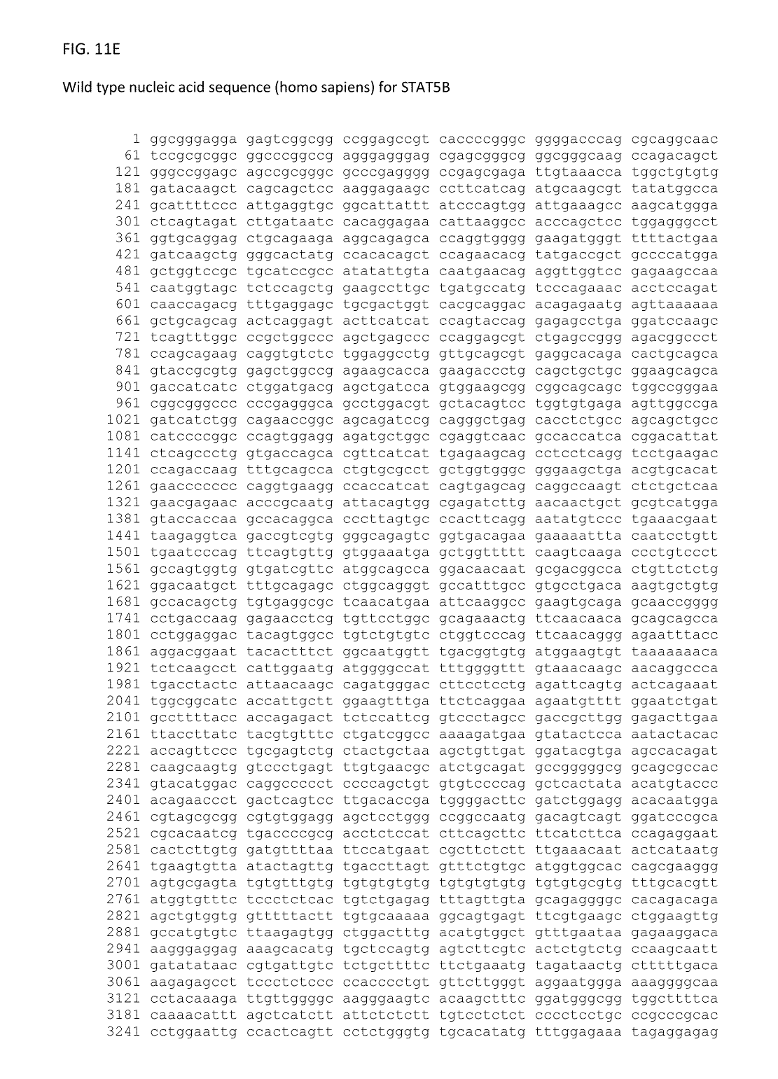

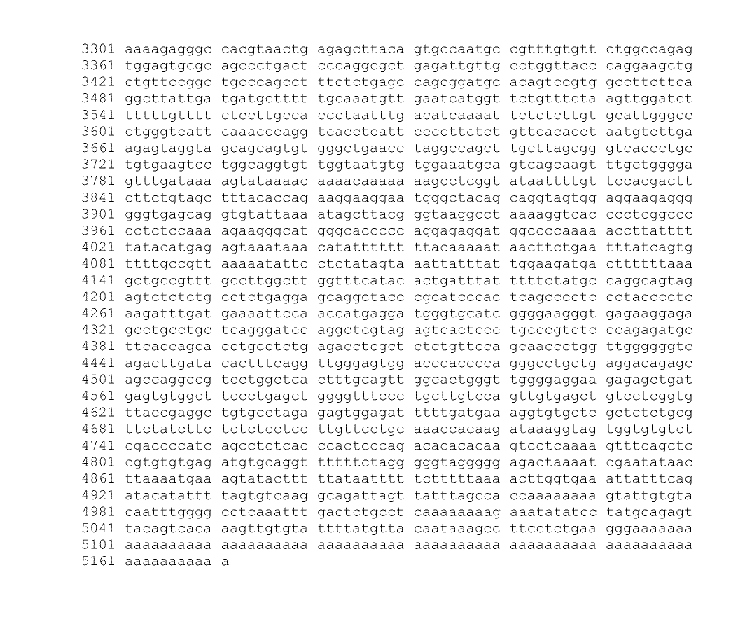

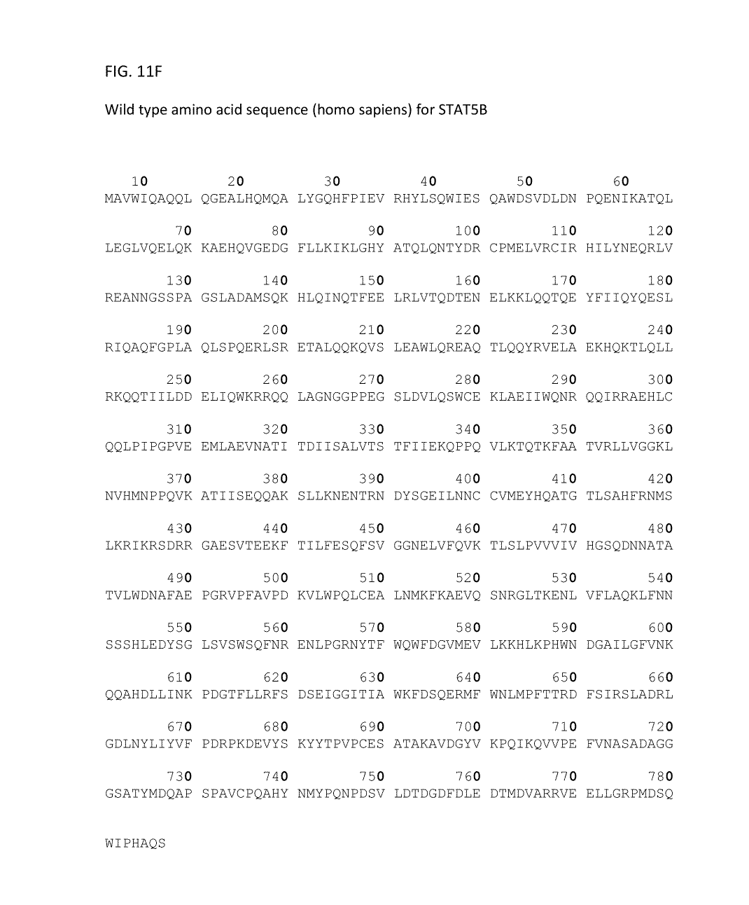

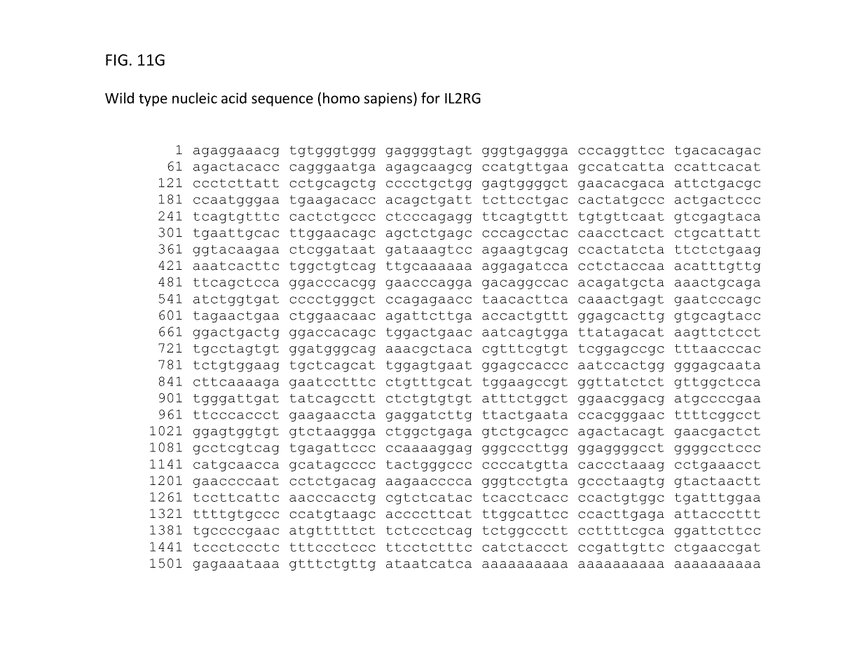

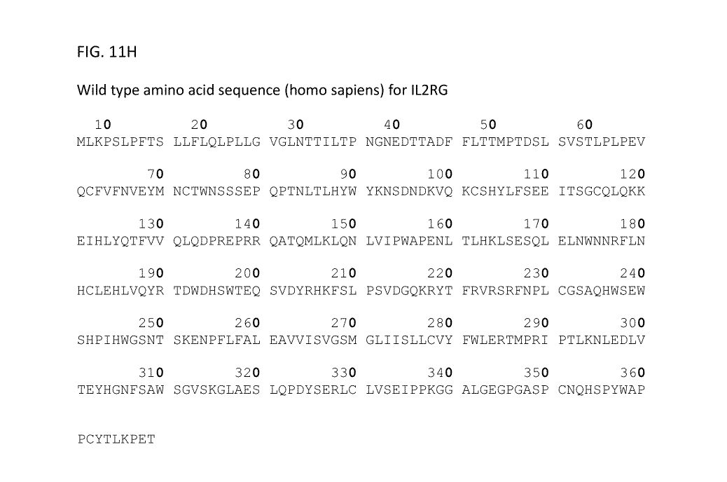

As used herein, the term “JAK/STAT pathway variant” refers to an aberrant or mutated or non-wild-type member of the JAK/STAT pathway. Examples of JAK/STAT pathway variants include any JAK1, JAK3, STAT5B, and/or IL2RG nucleic acid sequence and/or amino acid sequence differing in any manner from its respective wild type sequence. The homo sapiens wild type nucleic acid sequence and amino acid sequence for JAK1 is provided at FIGS. 11A and 11B, respectively. The homo sapiens wild type nucleic acid sequence and amino acid sequence for JAK3 is provided at FIGS. 11C and 11D, respectively. The homo sapiens wild type nucleic acid sequence and amino acid sequence for STAT5B is provided at FIGS. 121 and 121, respectively. The homo sapiens wild type nucleic acid sequence and amino acid sequence for IL2RG is provided at FIGS. 11G and 11H, respectively.

Specific examples of JAK1 variants include, but are not limited to, JAK1 polypeptides having an amino acid sequence differing from its respective wild type sequence (or nucleic acid sequence encoding such an amino acid) in the following manner: JAK1 p.F636L, JAK1 p.G646C, JAK1 p. Y654F, JAK1 p.V658F, JAK1 p.S703I, and JAK1 p.T901R. Specific examples of JAK3 variants include, but are not limited to, JAK3 polypeptides having an amino acid sequence differing from its respective wild type sequence (or nucleic acid sequence encoding such an amino acid) in the following manner: JAK3 p.M511I, JAK3 p. A573V, JAK3 p.R657., JAK3 p.G662W, JAK3 p.P664T, JAK3 p.Y980, JAK3 p.ΔKNC563, JAK3 p. Y981, and JAK3 p.S989I. Specific examples of STAT5B variants include, but are not limited to, STAT5B polypeptides having an amino acid sequence differing from its respective wild type sequence (or nucleic acid sequence encoding such an amino acid) in the following manner: STAT5B p.T628S, STAT5B p.N642H, STAT5B p.R659c, STAT5B p.Q706L, STAT5B p.Y699, and STAT5B p.Y665H. Specific examples of IL2RG variants include, but are not limited to, IL2RG polypeptides having an amino acid sequence differing from its respective wild type sequence (or nucleic acid sequence encoding such an amino acid) in the following manner: IL2RG p.ΔGSM268, 112RG p.Y325 and IL2RG p. K315E.

As used herein, the term “JAK inhibitor” refers to an agent (e.g., a pharmaceutical agent) that functions by inhibiting the activity of one or more of the Janus kinase (JAK) family of enzymes (e.g., JAK1, JAK2, JAK3, TYK2), thereby interfering with the JAK/STAT signaling pathway. Examples of JAK inhibitors include, but are not limited to, ruxolitinib, tofacitinib, baricitinib, CYT387, and lestaurtinib.

As used herein, the term “STAT inhibitor” refers to an agent (e.g., a pharmaceutical agent) that functions by inhibiting the activity of one or more of the Signal Transducer and Activator of Transcription (STAT) family, thereby interfering with the JAK/STAT signaling pathway. An example of a STAT inhibitor (e.g., STAT5B inhibitor) is pimozide.

As used herein, the term “biomarker” refers to an organic biomolecule which is differentially present in a sample taken from a subject of one phenotypic status (e.g. having a disease) as compared with another phenotypic status (e.g., not having the disease). A biomarker is differentially present between different phenotypic statuses if the mean or median expression level of the biomarker in the different groups is calculated to be statistically significant. In some embodiments, biomarkers, alone or in combination, provide measures of relative risk that a subject belongs to one phenotypic status or another. Therefore, they are useful as markers for disease (diagnostics), therapeutic effectiveness of a drug (theranostics) and drug toxicity.

Examples of mature T-cell leukemia biomarkers established through the experiments conducted during the present invention include, for example, variants of JAK1 having amino acid sequences differing its respective wild type sequence (e.g., JAK1 p.F636L, JAK1 p.G646C, JAK1 p. Y654F, JAK1 p.V658F, JAK1 p.S7031, and JAK1 p.T901R), variants of JAK3 having amino acid sequences differing its respective wild type sequence (e.g., JAK3 p.M511I, JAK3 p.ΔKNC563, JAK3 p. A573V, JAK3 p.G662W, JAK3 p.P664T, JAK3 p.R657. JAK3 p.Y980, JAK3 p. Y981, and JAK3 p.S989I), variants of STAT5B having amino acid sequences differing its respective wild type sequence (e.g., STAT5B p.T628S, STAT5B p.N642H, STAT5B p.Y699, STAT5B p.R659c, STAT5B p.Q706L, and STAT5B p.Y665H), and variants of IL2RG having amino acid sequences differing its respective wild type sequence (e.g., IL2RG p.ΔGSM268, 112RG p.Y325 and IL2RG p. K315E).

As used herein, the term “measuring” means methods which include detecting the presence or absence of biomarker(s) in the sample, quantifying the amount of marker(s) in the sample, and/or qualifying the type of biomarker. Measuring can be accomplished by methods known in the art and those further described herein. Any suitable methods can be used to detect and measure one or more of the markers described herein.

As used herein, the term “detect” refers to identifying the presence, absence or amount of the object to be detected (e.g., a biomarker).

As used herein, the term “diagnostic” means identifying the presence or nature of a pathologic condition, i.e., a mature T-cell leukemia (e.g., T-PLL, SS). Diagnostic methods differ in their sensitivity and specificity. As used herein, the term “sensitivity” is defined as a statistical measure of performance of an assay (e.g., method, test), calculated by dividing the number of true positives by the sum of the true positives and the false negatives. As used herein, the term “specificity” is defined as a statistical measure of performance of an assay (e.g., method, test), calculated by dividing the number of true negatives by the sum of true negatives and false positives.

As used herein, the term “informative” or “informativeness” refers to a quality of a marker or panel of markers, and specifically to the likelihood of finding a marker (or panel of markers) in a positive sample.

As used herein, the term “metastasis” is meant to refer to the process in which cancer cells originating in one organ or part of the body relocate to another part of the body and continue to replicate. Metastasized cells subsequently form tumors which may further metastasize. Metastasis thus refers to the spread of cancer from the part of the body where it originally occurs to other parts of the body.

The term “neoplasm” as used herein refers to any new and abnormal growth of tissue. Thus, a neoplasm can be a premalignant neoplasm or a malignant neoplasm. The term “neoplasm-specific marker” refers to any biological material that can be used to indicate the presence of a neoplasm. Examples of biological materials include, without limitation, nucleic acids, polypeptides, carbohydrates, fatty acids, cellular components (e.g., cell membranes and mitochondria), and whole cells.

As used herein, the term “adverse outcome” refers to an undesirable outcome in a patient diagnosed with a mature T-cell leukemia. In some embodiments, the patient is undergoing or has undergone treatment for a mature T-cell leukemia. Examples of adverse outcome include but are not limited to, recurrence of a mature T-cell leukemia, metastasis, transformation, or death.

As used herein, the term “amplicon” refers to a nucleic acid generated using primer pairs. The amplicon is typically single-stranded DNA (e.g., the result of asymmetric amplification), however, it may be RNA or dsDNA.

The term “amplifying” or “amplification” in the context of nucleic acids refers to the production of multiple copies of a polynucleotide, or a portion of the polynucleotide, typically starting from a small amount of the polynucleotide (e.g., a single polynucleotide molecule), where the amplification products or amplicons are generally detectable.

As used herein, the term “primer” refers to an oligonucleotide, whether occurring naturally as in a purified restriction digest or produced synthetically, that is capable of acting as a point of initiation of synthesis when placed under conditions in which synthesis of a primer extension product that is complementary to a nucleic acid strand is induced (e.g., in the presence of nucleotides and an inducing agent such as a biocatalyst (e.g., a DNA polymerase or the like) and at a suitable temperature and pH). The primer is typically single stranded for maximum efficiency in amplification, but may alternatively be double stranded. If double stranded, the primer is generally first treated to separate its strands before being used to prepare extension products. In some embodiments, the primer is an oligodeoxyribonucleotide. The primer is sufficiently long to prime the synthesis of extension products in the presence of the inducing agent. The exact lengths of the primers will depend on many factors, including temperature, source of primer and the use of the method. In certain embodiments, the primer is a capture primer.

A “sequence” of a biopolymer refers to the order and identity of monomer units (e.g., nucleotides, etc.) in the biopolymer. The sequence (e.g., base sequence) of a nucleic acid is typically read in the 5′ to 3′ direction.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.

As used herein, the term “non-human animals” refers to all non-human animals including, but are not limited to, vertebrates such as rodents, non-human primates, ovines, bovines, ruminants, lagomorphs, porcines, caprines, equines, canines, felines, ayes, etc.

As used therein, the term “locus” as used herein refers to a nucleic acid sequence on a chromosome or on a linkage map and includes the coding sequence as well as 5′ and 3′ sequences involved in regulation of the gene.

As used therein, the term “gas phase ion spectrometer” refers to an apparatus that detects gas phase ions. Gas phase ion spectrometers include an ion source that supplies gas phase ions. Gas phase ion spectrometers include, for example, mass spectrometers, ion mobility spectrometers, and total ion current measuring devices. “Gas phase ion spectrometry” refers to the use of a gas phase ion spectrometer to detect gas phase ions.

As used therein, the term “mass spectrometer” refers to a gas phase ion spectrometer that measures a parameter that can be translated into mass-to-charge ratios of gas phase ions. Mass spectrometers generally include an ion source and a mass analyzer. Examples of mass spectrometers are time-of-flight, magnetic sector, quadrupole filter, ion trap, ion cyclotron resonance, electrostatic sector analyzer and hybrids of these. “Mass spectrometry” refers to the use of a mass spectrometer to detect gas phase ions.

As used therein, the term “laser desorption mass spectrometer” refers to a mass spectrometer that uses laser energy as a means to desorb, volatilize, and ionize an analyte.

As used therein, the term “tandem mass spectrometer” refers to any mass spectrometer that is capable of performing two successive stages of m/z-based discrimination or measurement of ions, including ions in an ion mixture. The phrase includes mass spectrometers having two mass analyzers that are capable of performing two successive stages of m/z-based discrimination or measurement of ions tandem-in-space. The phrase further includes mass spectrometers having a single mass analyzer that is capable of performing two successive stages of m/z-based discrimination or measurement of ions tandem-in-time. The phrase thus explicitly includes Qq-TOF mass spectrometers, ion trap mass spectrometers, ion trap-TOF mass spectrometers, TOF-TOF mass spectrometers, Fourier transform ion cyclotron resonance mass spectrometers, electrostatic sector-magnetic sector mass spectrometers, and combinations thereof. As used therein, the term “mass analyzer” refers to a sub-assembly of a mass spectrometer that comprises means for measuring a parameter that can be translated into mass-to-charge ratios of gas phase ions. In a time-of-flight mass spectrometer the mass analyzer comprises an ion optic assembly, a flight tube and an ion detector.

As used therein, the term “ion source” refers to a sub-assembly of a gas phase ion spectrometer that provides gas phase ions. In one embodiment, the ion source provides ions through a desorption/ionization process. Such embodiments generally comprise a probe interface that positionally engages a probe in an interrogatable relationship to a source of ionizing energy (e.g., a laser desorption/ionization source) and in concurrent communication at atmospheric or subatmospheric pressure with a detector of a gas phase ion spectrometer Forms of ionizing energy for desorbing/ionizing an analyte from a solid phase include, for example: (1) laser energy; (2) fast atoms (used in fast atom bombardment); (3) high energy particles generated via beta decay of radionuclides (used in plasma desorption); and (4) primary ions generating secondary ions (used in secondary ion mass spectrometry). A preferred form of ionizing energy for solid phase analytes is a laser (used in laser desorption/ionization), in particular, nitrogen lasers, Nd-Yag lasers and other pulsed laser sources. “Fluence” refers to the energy delivered per unit area of interrogated image. A high fluence source, such as a laser, will deliver about 1 mJ/mm2 to 50 mJ/mm2. Typically, a sample is placed on the surface of a probe, the probe is engaged with the probe interface and the probe surface is struck with the ionizing energy. The energy desorbs analyte molecules from the surface into the gas phase and ionizes them. Other forms of ionizing energy for analytes include, for example: (1) electrons that ionize gas phase neutrals; (2) strong electric field to induce ionization from gas phase, solid phase, or liquid phase neutrals; and (3) a source that applies a combination of ionization particles or electric fields with neutral chemicals to induce chemical ionization of solid phase, gas phase, and liquid phase neutrals.

As used therein, the term “solid support” refers to a solid material which can be derivatized with, or otherwise attached to, a capture reagent. Exemplary solid supports include probes, microtiter plates and chromatographic resins.

As used therein, the term “probe” in the context of this invention refers to a device adapted to engage a probe interface of a gas phase ion spectrometer (e.g., a mass spectrometer) and to present an analyte to ionizing energy for ionization and introduction into a gas phase ion spectrometer, such as a mass spectrometer. A “probe” will generally comprise a solid substrate (either flexible or rigid) comprising a sample presenting surface on which an analyte is presented to the source of ionizing energy.

As used therein, the term “surface-enhanced laser desorption/ionization” or “SELDI” refers to a method of desorption/ionization gas phase ion spectrometry (e.g., mass spectrometry) in which the analyte is captured on the surface of a SELDI probe that engages the probe interface of the gas phase ion spectrometer. In “SELDI MS,” the gas phase ion spectrometer is a mass spectrometer. SELDI technology is described in, e.g., U.S. Pat. Nos. 5,719,060 and 6,225,047; each incorporated herein by reference in its entirety.

As used therein, the term “Surface-Enhanced Affinity Capture” or “SEAC” is a version of SELDI that involves the use of probes comprising an absorbent surface (a “SEAC probe”). “Adsorbent surface” refers to a surface to which is bound an adsorbent (also called a “capture reagent” or an “affinity reagent”). An adsorbent is any material capable of binding an analyte (e.g., a target polypeptide or nucleic acid). “Chromatographic adsorbent” refers to a material typically used in chromatography. Chromatographic adsorbents include, for example, ion exchange materials, metal chelators (e.g., nitriloacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents). “Biospecific adsorbent” refers an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047; incorporated herein by reference in its entirety. In some embodiments, a SEAC probe is provided as a pre-activated surface which can be modified to provide an adsorbent of choice. For example, certain probes are provided with a reactive moiety that is capable of binding a biological molecule through a covalent bond. Epoxide and carbodiimidizole are useful reactive moieties to covalently bind biospecific adsorbents such as antibodies or cellular receptors.

As used therein, the term “adsorption” refers to detectable non-covalent binding of an analyte to an adsorbent or capture reagent.

As used therein, the term “Surface-Enhanced Neat Desorption” or “SEND” is a version of SELDI that involves the use of probes comprising energy absorbing molecules chemically bound to the probe surface. (“SEND probe.”) “Energy absorbing molecules” (“EAM”) refer to molecules that are capable of absorbing energy from a laser desorption/ionization source and thereafter contributing to desorption and ionization of analyte molecules in contact therewith. The phrase includes molecules used in MALDI, frequently referred to as “matrix”, and explicitly includes cinnamic acid derivatives, sinapinic acid (“SPA”), cyano-hydroxy-cinnamic acid (“CHCA”) and dihydroxybenzoic acid, ferulic acid, hydroxyacetophenone derivatives, as well as others. It also includes EAMs used in SELDI.

As used therein, the term “Surface-Enhanced Photolabile Attachment and Release” or “SEPAR” is a version of SELDI that involves the use of probes having moieties attached to the surface that can covalently bind an analyte, and then release the analyte through breaking a photolabile bond in the moiety after exposure to light e.g., laser light. SEPAR is further described in U.S. Pat. No. 5,719,060; incorporated by reference in its entirety.

As used therein, the term “eluant” or “wash solution” refers to an agent, typically a solution, which is used to affect or modify adsorption of an analyte to an adsorbent surface and/or remove unbound materials from the surface. The elution characteristics of an eluant can depend, for example, on pH, ionic strength, hydrophobicity, degree of chaotropism, detergent strength and temperature.

As used herein, the term “biochip” refers to a solid substrate having a generally planar surface to which an adsorbent is attached. Frequently, the surface of the biochip comprises a plurality of addressable locations, each of which location has the adsorbent bound there. Biochips can be adapted to engage a probe interface and, therefore, function as probes.

As used herein, the term “protein biochip” refers to a biochip adapted for the capture of polypeptides. Many protein biochips are described in the art. These include, for example, protein biochips produced by Ciphergen Biosystems (Fremont, Calif.), Packard BioScience Company (Meriden Conn.), Zyomyx (Hayward, Calif.) and Phylos (Lexington, Mass.). Examples of such protein biochips are described in the following patents or patent applications: U.S. Pat. Nos. 6,329,209 and 6,225,047; International publication Nos. WO 99/51773 and WO 00/56934; each incorporated herein by reference in its entirety.

### DETAILED DESCRIPTION OF THE INVENTION

The molecular pathogenesis of mature T-cell leukemia (TCL) is poorly understood and current treatment options are limited to non-targeted cytotoxic agents. T-cell prolymphocytic leukemia (T-PLL) and Sezary syndrome (SS) are aggressive and chemotherapy-resistant neoplasia of mature T-lymphocytes characterized by a rapid clinical course and high mortality.

Experiments conducted during the course of developing embodiments for the present invention integrated mass spectrometry-driven phosphoproteomics with whole genome and exome sequencing to uncover oncogenic activation of the IL2R-JAK1/JAK3-STAT5B pathway in mature T-cell leukemias including T-PLL and SS. Phosphoproteomic and exome sequencing analysis of the SS-derived T-cell line HUT78 revealed activating phosphorylation of JAK3 and STAT5B and gain-of-function mutations in both JAK1 and JAK3. Whole genome sequencing of 4 index T-PLL samples identified JAK1 mutations not previously associated with T-PLL while WES of 39 additional T-PLL samples uncovered recurrently altered genes not previously implicated in T-PLL pathogenesis including CHEK2, EZH2 and FBXW10 in addition to frequent recurrent mutations targeting members of the JAK-STAT signaling pathway. Altogether, 38 of 50 T-PLL genomes (76.0%) harbored activating mutations in IL2RG, JAK1, JAK3 and STAT5B genes. Additional mutations in JAK1, JAK3 and STAT5B were also detected in SS (5/66, 7.6%). Functionally, expression of representative JAK1, JAK3 and STAT5b mutants exhibited STAT5 hyperactivation and/or IL-3 independent growth. Moreover, expression of recurrent STAT5B mutations exhibited enhanced colony formation. Importantly, such experiments further demonstrated constitutive activation of STAT5 in primary T-PLLs. Finally, targeted STAT5B inhibition reduced pSTAT5B levels in primary T-PLL cells with consequent apoptotic cell death. Altogether, these results implicate recurrent mutational activation of cytokine receptor-JAK-STAT signaling in the pathogenesis of mature T-cell leukemias and suggest opportunities for more effective and less toxic novel therapies targeting the JAK-STAT pathway in these aggressive cancers.

Accordingly, the present invention provides methods and biomarkers for detection and characterization of mature T-cell neoplasias/leukemias (e.g., T-cell prolymphocytic leukemia, Sezary syndrome) in biological samples (e.g., tissue samples, blood samples, plasma samples, cell samples, serum samples).

### I. Diagnostic and Screening Applications

Embodiments of the present invention provide diagnostic, prognostic, and screening methods. In some embodiments, the methods characterize and diagnose mature T-cell leukemias (e.g., T-cell prolymphocytic leukemia (T-PLL), Sezary syndrome (SS), mycosis fungoides (MF)) through detection and/or characterization of aberrant JAK/STAT pathway activity within T-cells from a biological sample. Examples of mature T-cell leukemias include T-cell prolymphocytic leukemia (T-PLL), Sezary syndrome (SS), and mycosis fungoides (MF). Exemplary, non-limiting methods of identifying aberrant JAK/STAT pathway activity within T-cells from a biological are described below.

### A. JAK/STAT Signaling Pathway Mutations

Embodiments of the present invention provide compositions and methods for detecting JAK/STAT pathway mutations (e.g., to identify or diagnose T-cell leukemias). The present invention is not limited to particular JAK/STAT pathway mutations. In some embodiments, mutations are loss of function mutations (e.g., truncation, nonsense, missense, or frameshift mutations) and/or gain of function mutations.

Exemplary mutations include, but are not limited to, any nucleic acid and/or polypeptide mutation related to the JAK/STAT pathway (e.g., nucleic acid and/or polypeptide mutations related to JAK1, JAK3, STAT5B, IL2RG). Examples of JAK1 mutations include, for example, JAK1 polypeptides having an amino acid differing from its respective wild type sequence (and nucleic acid sequence encoding such amino acid sequence) (e.g., JAK1 p.F636L, JAK1 p.G646C, JAK1 p. Y654F, JAK1 p.V658F, JAK1 p.S703I, and JAK1 p.T901R). Examples of JAK3 mutations include, for example, JAK3 polypeptides having an amino acid differing from its respective wild type sequence (and nucleic acid sequence encoding such amino acid sequence) (e.g., JAK3 p.G662W, JAK3 p.P664T, JAK3 p.M511I, JAK3 p. A573V, JAK3 p.R657., JAK3 p.ΔKNC563, JAK3 p.Y980, JAK3 p. Y981, and JAK3 p.S989I). Examples of STAT5B mutations include, for example, STAT5B polypeptides having an amino acid differing from its respective wild type sequence (and nucleic acid sequence encoding such amino acid sequence) (e.g., STAT5B p.T628S, STAT5B p.N642H, STAT5B p.Y699, STAT5B p.R659c, STAT5B p.Q706L, and STAT5B p.Y665H). Examples of IL2RG mutations include, for example, IL2RG polypeptides having an amino acid differing from its respective wild type sequence (and nucleic acid sequence encoding such amino acid sequence) (e.g., IL2RG p.ΔGSM268, 112RG p.Y325 and IL2RG p. K315E).

In some embodiments, detection of one or more of the following JAK/STAT pathway mutations (in comparison to wild type) is used to identify or diagnose T-cell prolymphocytic leukemia: JAK1 p.V658F, JAK1 p.S703I, JAK1 p.T901R, JAK3 p.ΔKNC563, JAK3 p.M511I, JAK3 p. A573V, JAK3 p.R657, STAT5B p.T628S, STAT5B p.R659c, STAT5B p.Q706L, STAT5B p.N642H, STAT5B p.Y665H, IL2RG p.ΔGSM268, and IL2RG p. K315E.

In some embodiments, detection of one or more of the following JAK/STAT pathway mutations (in comparison to wild type) is used to identify or diagnose Sezary syndrome: JAK1 p. Y654F, JAK3 p. A573V, JAK3 p.Y980, JAK3 p. Y981, JAK3 p.S989I, STAT5B p.Y699, STAT5B p.N642H, and I12RG p.Y325.

In some embodiments, detection of one or more of the following JAK/STAT pathway mutations (in comparison to wild type) is used to identify or diagnose mycosis fungoides: JAK1 p.F636L, JAK1 p.G646C, JAK3 p.G662W, and JAK3 p.P664T.

While the present invention exemplifies several markers specific for detecting mature T-cell leukemias (e.g., T-PLL, SS, MF), any marker that is correlated with the presence or absence or prognosis of mature T-cell leukemias may be used. A marker, as used herein, includes, for example, nucleic acid(s) whose production or mutation or lack of production is characteristic of a mature T-cell leukemia and mutations that cause the same effect (e.g., deletions, truncations, etc).

In some embodiments, one or more (e.g., 2, 3, 4, 5, 6, 7, 8, 9, 10, 15, 20, 25, or more (e.g., all)) of the mutations are identified in order to diagnose or characterize a mature T-cell leukemia (e.g., T-PLL, SS, MF). In some embodiments, multiple markers are detected in a panel or multiplex format.

Particular combinations of markers may be used that show optimal function with different ethnic groups or sex, different geographic distributions, different stages of disease, different degrees of specificity or different degrees of sensitivity. Particular combinations may also be developed which are particularly sensitive to the effect of therapeutic regimens on disease progression. Subjects may be monitored after a therapy and/or course of action to determine the effectiveness of that specific therapy and/or course of action.

### B. Detection of Alleles

In some embodiments, the present invention provides methods of detecting the presence of wild type or variant (e.g., mutant or polymorphic) nucleic acids or polypeptides related to the JAK/STAT pathway (e.g., JAK1, JAK3, STAT5B, IL2RG). The detection of mutant nucleic acids or polypeptides related to the JAK/STAT pathway finds use in the diagnosis of disease (e.g., mature T-cell leukemias), research, and selection of appropriate treatment and/or monitoring regimens.

Accordingly, the present invention provides methods for determining whether a subject (e.g., a human patient) has a JAK/STAT pathway related mutation profile associated with a mature T-cell leukemia (e.g., T-PLL, SS, MF).

A number of methods are available for analysis of variant (e.g., mutant or polymorphic) nucleic acid sequences. Assays for detecting variants (e.g., polymorphisms or mutations) fall into several categories, including, but not limited to direct sequencing assays, fragment polymorphism assays, hybridization assays, and computer based data analysis. Protocols and commercially available kits or services for performing multiple variations of these assays are available. In some embodiments, assays are performed in combination or in hybrid (e.g., different reagents or technologies from several assays are combined to yield one assay). The following assays are useful in the present invention.

Any patient sample containing JAK/STAT pathway nucleic acids or polypeptides (e.g., JAK1, JAK3, STAT5B, IL2RG) may be tested according to the methods of the present invention. By way of non-limiting examples, the sample may be tissue, blood, urine, semen, or a fraction thereof (e.g., plasma, serum, whole blood, spleen cells, etc.).

The patient sample may undergo preliminary processing designed to isolate or enrich the sample for the JAK/STAT pathway nucleic acids or polypeptides (e.g., JAK1, JAK3, STAT5B, IL2RG) or cells that contain such nucleic acids or polypeptides. A variety of techniques known to those of ordinary skill in the art may be used for this purpose, including but not limited: centrifugation; immunocapture; cell lysis; and, nucleic acid target capture (See, e.g., EP Pat. No. 1 409 727, herein incorporated by reference in its entirety).

i. DNA and RNA Detection

Variant JAK/STAT pathway nucleic acids or polypeptides (e.g., JAK1, JAK3, STAT5B, IL2RG) of the present invention may be detected as genomic DNA or mRNA using a variety of nucleic acid techniques known to those of ordinary skill in the art, including but not limited to: nucleic acid sequencing; nucleic acid hybridization; and, nucleic acid amplification.

1. Sequencing

Illustrative non-limiting examples of nucleic acid sequencing techniques include, but are not limited to, chain terminator (Sanger) sequencing and dye terminator sequencing. Those of ordinary skill in the art will recognize that because RNA is less stable in the cell and more prone to nuclease attack experimentally RNA is usually reverse transcribed to DNA before sequencing.

Chain terminator sequencing uses sequence-specific termination of a DNA synthesis reaction using modified nucleotide substrates. Extension is initiated at a specific site on the template DNA by using a short radioactive, fluorescent or other labeled, oligonucleotide primer complementary to the template at that region. The oligonucleotide primer is extended using a DNA polymerase, standard four deoxynucleotide bases, and a low concentration of one chain terminating nucleotide, most commonly a di-deoxynucleotide. This reaction is repeated in four separate tubes with each of the bases taking turns as the di-deoxynucleotide. Limited incorporation of the chain terminating nucleotide by the DNA polymerase results in a series of related DNA fragments that are terminated only at positions where that particular di-deoxynucleotide is used. For each reaction tube, the fragments are size-separated by electrophoresis in a slab polyacrylamide gel or a capillary tube filled with a viscous polymer. The sequence is determined by reading which lane produces a visualized mark from the labeled primer as you scan from the top of the gel to the bottom.

Dye terminator sequencing alternatively labels the terminators. Complete sequencing can be performed in a single reaction by labeling each of the di-deoxynucleotide chain-terminators with a separate fluorescent dye, which fluoresces at a different wavelength.

Some embodiments of the present invention utilize next generation or high-throughput sequencing. A variety of nucleic acid sequencing methods are contemplated for use in the methods of the present disclosure including, for example, chain terminator (Sanger) sequencing, dye terminator sequencing, and high-throughput sequencing methods. Many of these sequencing methods are well known in the art. See, e.g., Sanger et al., Proc. Natl. Acad. Sci. USA 74:5463-5467 (1997); Maxam et al., Proc. Natl. Acad. Sci. USA 74:560-564 (1977); Drmanac, et al., Nat. Biotechnol. 16:54-58 (1998); Kato, Int. J. Clin. Exp. Med. 2:193-202 (2009); Ronaghi et al., Anal. Biochem. 242:84-89 (1996); Margulies et al., Nature 437:376-380 (2005); Ruparel et al., Proc. Natl. Acad. Sci. USA 102:5932-5937 (2005), and Harris et al., Science 320:106-109 (2008); Levene et al., Science 299:682-686 (2003); Korlach et al., Proc. Natl. Acad. Sci. USA 105:1176-1181 (2008); Branton et al., Nat. Biotechnol. 26(10):1146-53 (2008); Eid et al., Science 323:133-138 (2009); each of which is herein incorporated by reference in its entirety.

In some embodiments, sequencing technology including, but not limited to, pyrosequencing, sequencing-by-ligation, single molecule sequencing, sequence-by-synthesis (SBS), massive parallel clonal, massive parallel single molecule SBS, massive parallel single molecule real-time, massive parallel single molecule real-time nanopore technology, etc. Morozova and Marra provide a review of some such technologies in Genomics, 92: 255 (2008), herein incorporated by reference in its entirety. Those of ordinary skill in the art will recognize that because RNA is less stable in the cell and more prone to nuclease attack experimentally RNA is usually reverse transcribed to DNA before sequencing.

A number of DNA sequencing techniques are known in the art, including fluorescence-based sequencing methodologies (see, e.g., Birren et al., Genome Analysis: Analyzing DNA, 1, Cold Spring Harbor, N.Y.; herein incorporated by reference in its entirety). In some embodiments, the technology finds use in automated sequencing techniques understood in that art. In some embodiments, the present technology finds use in parallel sequencing of partitioned amplicons (PCT Publication No: WO2006084132 to Kevin McKernan et al., herein incorporated by reference in its entirety). In some embodiments, the technology finds use in DNA sequencing by parallel oligonucleotide extension (See, e.g., U.S. Pat. No. 5,750,341 to Macevicz et al., and U.S. Pat. No. 6,306,597 to Macevicz et al., both of which are herein incorporated by reference in their entireties). Additional examples of sequencing techniques in which the technology finds use include the Church polony technology (Mitra et al., 2003, Analytical Biochemistry 320, 55-65; Shendure et al., 2005 Science 309, 1728-1732; U.S. Pat. No. 6,432,360, U.S. Pat. No. 6,485,944, U.S. Pat. No. 6,511,803; herein incorporated by reference in their entireties), the 454 picotiter pyrosequencing technology (Margulies et al., 2005 Nature 437, 376-380; US 20050130173; herein incorporated by reference in their entireties), the Solexa single base addition technology (Bennett et al., 2005, Pharmacogenomics, 6, 373-382; U.S. Pat. No. 6,787,308; U.S. Pat. No. 6,833,246; herein incorporated by reference in their entireties), the Lynx massively parallel signature sequencing technology (Brenner et al. (2000). Nat. Biotechnol. 18:630-634; U.S. Pat. No. 5,695,934; U.S. Pat. No. 5,714,330; herein incorporated by reference in their entireties), and the Adessi PCR colony technology (Adessi et al. (2000). Nucleic Acid Res. 28, E87; WO 00018957; herein incorporated by reference in its entirety).

Next-generation sequencing (NGS) methods share the common feature of massively parallel, high-throughput strategies, with the goal of lower costs in comparison to older sequencing methods (see, e.g., Voelkerding et al., Clinical Chem., 55: 641-658, 2009; MacLean et al., Nature Rev. Microbiol., 7: 287-296; each herein incorporated by reference in their entirety). NGS methods can be broadly divided into those that typically use template amplification and those that do not. Amplification-requiring methods include pyrosequencing commercialized by Roche as the 454 technology platforms (e.g., GS 20 and GS FLX), the Solexa platform commercialized by Illumina, and the Supported Oligonucleotide Ligation and Detection (SOLiD) platform commercialized by Applied Biosystems. Non-amplification approaches, also known as single-molecule sequencing, are exemplified by the HeliScope platform commercialized by Helicos BioSciences, and emerging platforms commercialized by VisiGen, Oxford Nanopore Technologies Ltd., Life Technologies/Ion Torrent, and Pacific Biosciences, respectively.

In pyrosequencing (Voelkerding et al., Clinical Chem., 55: 641-658, 2009; MacLean et al., Nature Rev. Microbiol., 7: 287-296; U.S. Pat. No. 6,210,891; U.S. Pat. No. 6,258,568; each herein incorporated by reference in its entirety), template DNA is fragmented, end-repaired, ligated to adaptors, and clonally amplified in-situ by capturing single template molecules with beads bearing oligonucleotides complementary to the adaptors. Each bead bearing a single template type is compartmentalized into a water-in-oil microvesicle, and the template is clonally amplified using a technique referred to as emulsion PCR. The emulsion is disrupted after amplification and beads are deposited into individual wells of a picotitre plate functioning as a flow cell during the sequencing reactions. Ordered, iterative introduction of each of the four dNTP reagents occurs in the flow cell in the presence of sequencing enzymes and luminescent reporter such as luciferase. In the event that an appropriate dNTP is added to the 3′ end of the sequencing primer, the resulting production of ATP causes a burst of luminescence within the well, which is recorded using a CCD camera. It is possible to achieve read lengths greater than or equal to 400 bases, and 10⁶ sequence reads can be achieved, resulting in up to 500 million base pairs (Mb) of sequence.

In the Solexa/Illumina platform (Voelkerding et al., Clinical Chem., 55: 641-658, 2009; MacLean et al., Nature Rev. Microbiol., 7: 287-296; U.S. Pat. No. 6,833,246; U.S. Pat. No. 7,115,400; U.S. Pat. No. 6,969,488; each herein incorporated by reference in its entirety), sequencing data are produced in the form of shorter-length reads. In this method, single-stranded fragmented DNA is end-repaired to generate 5′-phosphorylated blunt ends, followed by Klenow-mediated addition of a single A base to the 3′ end of the fragments. A-addition facilitates addition of T-overhang adaptor oligonucleotides, which are subsequently used to capture the template-adaptor molecules on the surface of a flow cell that is studded with oligonucleotide anchors. The anchor is used as a PCR primer, but because of the length of the template and its proximity to other nearby anchor oligonucleotides, extension by PCR results in the “arching over” of the molecule to hybridize with an adjacent anchor oligonucleotide to form a bridge structure on the surface of the flow cell. These loops of DNA are denatured and cleaved. Forward strands are then sequenced with reversible dye terminators. The sequence of incorporated nucleotides is determined by detection of post-incorporation fluorescence, with each fluor and block removed prior to the next cycle of dNTP addition. Sequence read length ranges from 36 nucleotides to over 50 nucleotides, with overall output exceeding 1 billion nucleotide pairs per analytical run.

Sequencing nucleic acid molecules using SOLiD technology (Voelkerding et al., Clinical Chem., 55: 641-658, 2009; MacLean et al., Nature Rev. Microbiol., 7: 287-296; U.S. Pat. No. 5,912,148; U.S. Pat. No. 6,130,073; each herein incorporated by reference in their entirety) also involves fragmentation of the template, ligation to oligonucleotide adaptors, attachment to beads, and clonal amplification by emulsion PCR. Following this, beads bearing template are immobilized on a derivatized surface of a glass flow-cell, and a primer complementary to the adaptor oligonucleotide is annealed. However, rather than utilizing this primer for 3′ extension, it is instead used to provide a 5′ phosphate group for ligation to interrogation probes containing two probe-specific bases followed by 6 degenerate bases and one of four fluorescent labels. In the SOLiD system, interrogation probes have 16 possible combinations of the two bases at the 3′ end of each probe, and one of four fluors at the 5′ end. Fluor color, and thus identity of each probe, corresponds to specified color-space coding schemes. Multiple rounds (usually 7) of probe annealing, ligation, and fluor detection are followed by denaturation, and then a second round of sequencing using a primer that is offset by one base relative to the initial primer. In this manner, the template sequence can be computationally re-constructed, and template bases are interrogated twice, resulting in increased accuracy. Sequence read length averages 35 nucleotides, and overall output exceeds 4 billion bases per sequencing run.

In certain embodiments, the technology finds use in nanopore sequencing (see, e.g., Astier et al., J. Am. Chem. Soc. 2006 Feb. 8; 128(5):1705-10, herein incorporated by reference). The theory behind nanopore sequencing has to do with what occurs when a nanopore is immersed in a conducting fluid and a potential (voltage) is applied across it. Under these conditions a slight electric current due to conduction of ions through the nanopore can be observed, and the amount of current is exceedingly sensitive to the size of the nanopore. As each base of a nucleic acid passes through the nanopore, this causes a change in the magnitude of the current through the nanopore that is distinct for each of the four bases, thereby allowing the sequence of the DNA molecule to be determined.

In certain embodiments, the technology finds use in HeliScope by Helicos BioSciences (Voelkerding et al., Clinical Chem., 55: 641-658, 2009; MacLean et al., Nature Rev. Microbiol., 7: 287-296; U.S. Pat. No. 7,169,560; U.S. Pat. No. 7,282,337; U.S. Pat. No. 7,482,120; U.S. Pat. No. 7,501,245; U.S. Pat. No. 6,818,395; U.S. Pat. No. 6,911,345; U.S. Pat. No. 7,501,245; each herein incorporated by reference in their entirety). Template DNA is fragmented and polyadenylated at the 3′ end, with the final adenosine bearing a fluorescent label. Denatured polyadenylated template fragments are ligated to poly(dT) oligonucleotides on the surface of a flow cell. Initial physical locations of captured template molecules are recorded by a CCD camera, and then label is cleaved and washed away. Sequencing is achieved by addition of polymerase and serial addition of fluorescently-labeled dNTP reagents. Incorporation events result in fluor signal corresponding to the dNTP, and signal is captured by a CCD camera before each round of dNTP addition. Sequence read length ranges from 25-50 nucleotides, with overall output exceeding 1 billion nucleotide pairs per analytical run.

The Ion Torrent technology is a method of DNA sequencing based on the detection of hydrogen ions that are released during the polymerization of DNA (see, e.g., Science 327(5970): 1190 (2010); U.S. Pat. Appl. Pub. Nos. 20090026082, 20090127589, 20100301398, 20100197507, 20100188073, and 20100137143, incorporated by reference in their entireties for all purposes). A microwell contains a template DNA strand to be sequenced. Beneath the layer of microwells is a hypersensitive ISFET ion sensor. All layers are contained within a CMOS semiconductor chip, similar to that used in the electronics industry. When a dNTP is incorporated into the growing complementary strand a hydrogen ion is released, which triggers a hypersensitive ion sensor. If homopolymer repeats are present in the template sequence, multiple dNTP molecules will be incorporated in a single cycle. This leads to a corresponding number of released hydrogens and a proportionally higher electronic signal. This technology differs from other sequencing technologies in that no modified nucleotides or optics are used. The per-base accuracy of the Ion Torrent sequencer is ˜99.6% for 50 base reads, with ˜100 Mb generated per run. The read-length is 100 base pairs. The accuracy for homopolymer repeats of 5 repeats in length is ˜98%. The benefits of ion semiconductor sequencing are rapid sequencing speed and low upfront and operating costs.

The technology finds use in another nucleic acid sequencing approach developed by Stratos Genomics, Inc. and involves the use of Xpandomers. This sequencing process typically includes providing a daughter strand produced by a template-directed synthesis. The daughter strand generally includes a plurality of subunits coupled in a sequence corresponding to a contiguous nucleotide sequence of all or a portion of a target nucleic acid in which the individual subunits comprise a tether, at least one probe or nucleobase residue, and at least one selectively cleavable bond. The selectively cleavable bond(s) is/are cleaved to yield an Xpandomer of a length longer than the plurality of the subunits of the daughter strand. The Xpandomer typically includes the tethers and reporter elements for parsing genetic information in a sequence corresponding to the contiguous nucleotide sequence of all or a portion of the target nucleic acid. Reporter elements of the Xpandomer are then detected. Additional details relating to Xpandomer-based approaches are described in, for example, U.S. Pat. Pub No. 20090035777, entitled “High Throughput Nucleic Acid Sequencing by Expansion,” filed Jun. 19, 2008, which is incorporated herein in its entirety.

Other emerging single molecule sequencing methods include real-time sequencing by synthesis using a VisiGen platform (Voelkerding et al., Clinical Chem., 55: 641-58, 2009; U.S. Pat. No. 7,329,492; U.S. patent application Ser. No. 11/671,956; U.S. patent application Ser. No. 11/781,166; each herein incorporated by reference in their entirety) in which immobilized, primed DNA template is subjected to strand extension using a fluorescently-modified polymerase and florescent acceptor molecules, resulting in detectible fluorescence resonance energy transfer (FRET) upon nucleotide addition.

In some embodiments, capillary electrophoresis (CE) is utilized to analyze amplification fragments. During capillary electrophoresis, nucleic acids (e.g., the products of a PCR reaction) are injected electrokinetically into capillaries filled with polymer. High voltage is applied so that the fluorescent DNA fragments are separated by size and are detected by a laser/camera system. In some embodiments, CE systems from Life Technogies (Grand Island, N.Y.) are utilized for fragment sizing (see e.g., U.S. Pat. Nos. 6,706,162, 8,043,493, each of which is herein incorporated by reference in its entirety).

2. Hybridization

Illustrative non-limiting examples of nucleic acid hybridization techniques include, but are not limited to, in situ hybridization (ISH), microarray, and Southern or Northern blot. In situ hybridization (ISH) is a type of hybridization that uses a labeled complementary DNA or RNA strand as a probe to localize a specific DNA or RNA sequence in a portion or section of tissue (in situ), or, if the tissue is small enough, the entire tissue (whole mount ISH). DNA ISH can be used to determine the structure of chromosomes. RNA ISH is used to measure and localize mRNAs and other transcripts within tissue sections or whole mounts. Sample cells and tissues are usually treated to fix the target transcripts in place and to increase access of the probe. The probe hybridizes to the target sequence at elevated temperature, and then the excess probe is washed away. The probe that was labeled with either radio-, fluorescent- or antigen-labeled bases is localized and quantitated in the tissue using either autoradiography, fluorescence microscopy or immunohistochemistry, respectively. ISH can also use two or more probes, labeled with radioactivity or the other non-radioactive labels, to simultaneously detect two or more transcripts.

In some embodiments, the present invention provides nucleic acid probes specific for a particular JAK/STAT pathway variant. For example, in some embodiments, separate nucleic acid probes are provided that are only specific for one JAK/STAT pathway variant as described herein (e.g., the nucleic acid encoding any JAK/STAT pathway variant including, but not limited to, JAK1 p.F636L, JAK1 p.G646C, JAK1 p. Y654F, JAK1 p.V658F, JAK1 p.S7031, JAK1 p.T901R, JAK3 p.G662W, JAK3 p.P664T, JAK3 p.ΔKNC563, JAK3 p.M511I, JAK3 p. A573V, JAK3 p.R657. JAK3 p.Y980, JAK3 p. Y981, and JAK3 p.S989I, STAT5B p.T628S, STAT5B p.R659c, STAT5B p.Q706L, STAT5B p.N642H, STAT5B p.Y699, and STAT5B p.Y665H, IL2RG p.Y325, IL2RG p.ΔGSM268, and IL2RG p. K315E). In some embodiments, such separate nucleic acid probes are specific for the entire JAK/STAT pathway variant. In some embodiments, such separate nucleic acid probes are specific for a nucleic acid fragment of such a JAK/STAT pathway variant. In some embodiments, such separate nucleic acid probes specific for a JAK/STAT pathway variant will not bind the respective wild type equivalent JAK/STAT pathway variant. In some embodiments, such separate nucleic acid probes specific for a JAK/STAT pathway variant will not bind different JAK/STAT pathway variants.

3. Microarrays

In some embodiments, microarrays are utilized for detection of JAK/STAT pathway nucleic acid (e.g., JAK1, JAK3, STAT5B, IL2RG) sequences. Examples of microarrays include, but not limited to: DNA microarrays (e.g., cDNA microarrays and oligonucleotide microarrays); protein microarrays; tissue microarrays; transfection or cell microarrays; chemical compound microarrays; and, antibody microarrays. A DNA microarray, commonly known as gene chip, DNA chip, or biochip, is a collection of microscopic DNA spots attached to a solid surface (e.g., glass, plastic or silicon chip) forming an array for the purpose of expression profiling or monitoring expression levels for thousands of genes simultaneously. The affixed DNA segments are known as probes, thousands of which can be used in a single DNA microarray. Microarrays can be used to identify disease genes by comparing gene expression in disease and normal cells. Microarrays can be fabricated using a variety of technologies, including but not limiting: printing with fine-pointed pins onto glass slides; photolithography using pre-made masks; photolithography using dynamic micromirror devices; ink jet printing; or, electrochemistry on microelectrode arrays.

Arrays can also be used to detect copy number variations at a specific locus. These genomic micorarrys detect microscopic deletions or other variants that lead to disease causing alleles.

Southern and Northern blotting is used to detect specific DNA or RNA sequences, respectively. DNA or RNA extracted from a sample is fragmented, electrophoretically separated on a matrix gel, and transferred to a membrane filter. The filter bound DNA or RNA is subject to hybridization with a labeled probe complementary to the sequence of interest. Hybridized probe bound to the filter is detected. A variant of the procedure is the reverse Northern blot, in which the substrate nucleic acid that is affixed to the membrane is a collection of isolated DNA fragments and the probe is RNA extracted from a tissue and labeled.

4. Amplification

JAK/STAT pathway (e.g., JAK1, JAK3, STAT5B, IL2RG) nucleic acid may be amplified prior to or simultaneous with detection. Illustrative non-limiting examples of nucleic acid amplification techniques include, but are not limited to, polymerase chain reaction (PCR), reverse transcription polymerase chain reaction (RT-PCR), transcription-mediated amplification (TMA), ligase chain reaction (LCR), strand displacement amplification (SDA), and nucleic acid sequence based amplification (NASBA). Those of ordinary skill in the art will recognize that certain amplification techniques (e.g., PCR) require that RNA be reversed transcribed to DNA prior to amplification (e.g., RT-PCR), whereas other amplification techniques directly amplify RNA (e.g., TMA and NASBA).

The polymerase chain reaction (U.S. Pat. Nos. 4,683,195, 4,683,202, 4,800,159 and 4,965,188, each of which is herein incorporated by reference in its entirety), commonly referred to as PCR, uses multiple cycles of denaturation, annealing of primer pairs to opposite strands, and primer extension to exponentially increase copy numbers of a target nucleic acid sequence. In a variation called RT-PCR, reverse transcriptase (RT) is used to make a complementary DNA (cDNA) from mRNA, and the cDNA is then amplified by PCR to produce multiple copies of DNA. For other various permutations of PCR see, e.g., U.S. Pat. Nos. 4,683,195, 4,683,202 and 4,800,159; Mullis et al., Meth. Enzymol. 155: 335 (1987); and, Murakawa et al., DNA 7: 287 (1988), each of which is herein incorporated by reference in its entirety.

Transcription mediated amplification (U.S. Pat. Nos. 5,480,784 and 5,399,491, each of which is herein incorporated by reference in its entirety), commonly referred to as TMA, synthesizes multiple copies of a target nucleic acid sequence autocatalytically under conditions of substantially constant temperature, ionic strength, and pH in which multiple RNA copies of the target sequence autocatalytically generate additional copies. See, e.g., U.S. Pat. Nos. 5,399,491 and 5,824,518, each of which is herein incorporated by reference in its entirety. In a variation described in U.S. Publ. No. 20060046265 (herein incorporated by reference in its entirety), TMA optionally incorporates the use of blocking moieties, terminating moieties, and other modifying moieties to improve TMA process sensitivity and accuracy.

The ligase chain reaction (Weiss, R., Science 254: 1292 (1991), herein incorporated by reference in its entirety), commonly referred to as LCR, uses two sets of complementary DNA oligonucleotides that hybridize to adjacent regions of the target nucleic acid. The DNA oligonucleotides are covalently linked by a DNA ligase in repeated cycles of thermal denaturation, hybridization and ligation to produce a detectable double-stranded ligated oligonucleotide product.

Strand displacement amplification (Walker, G. et al., Proc. Natl. Acad. Sci. USA 89: 392-396 (1992); U.S. Pat. Nos. 5,270,184 and 5,455,166, each of which is herein incorporated by reference in its entirety), commonly referred to as SDA, uses cycles of annealing pairs of primer sequences to opposite strands of a target sequence, primer extension in the presence of a dNTPαS to produce a duplex hemiphosphorothioated primer extension product, endonuclease-mediated nicking of a hemimodified restriction endonuclease recognition site, and polymerase-mediated primer extension from the 3′ end of the nick to displace an existing strand and produce a strand for the next round of primer annealing, nicking and strand displacement, resulting in geometric amplification of product. Thermophilic SDA (tSDA) uses thermophilic endonucleases and polymerases at higher temperatures in essentially the same method (EP Pat. No. 0 684 315).

Other amplification methods include, for example: nucleic acid sequence based amplification (U.S. Pat. No. 5,130,238, herein incorporated by reference in its entirety), commonly referred to as NASBA; one that uses an RNA replicase to amplify the probe molecule itself (Lizardi et al., BioTechnol. 6: 1197 (1988), herein incorporated by reference in its entirety), commonly referred to as Qβ replicase; a transcription based amplification method (Kwoh et al., Proc. Natl. Acad. Sci. USA 86:1173 (1989)); and, self-sustained sequence replication (Guatelli et al., Proc. Natl. Acad. Sci. USA 87: 1874 (1990), each of which is herein incorporated by reference in its entirety). For further discussion of known amplification methods see Persing, David H., “In Vitro Nucleic Acid Amplification Techniques” in Diagnostic Medical Microbiology: Principles and Applications (Persing et al., Eds.), pp. 51-87 (American Society for Microbiology, Washington, D.C. (1993)).

5. Detection Methods

Non-amplified or amplified JAK/STAT pathway (e.g., JAK1, JAK3, STAT5B, IL2RG) nucleic acids can be detected by any conventional means. For example, nucleic acid can be detected by hybridization with a detectably labeled probe and measurement of the resulting hybrids. Illustrative non-limiting examples of detection methods are described below.

One illustrative detection method, the Hybridization Protection Assay (HPA) involves hybridizing a chemiluminescent oligonucleotide probe (e.g., an acridinium ester-labeled (AE) probe) to the target sequence, selectively hydrolyzing the chemiluminescent label present on unhybridized probe, and measuring the chemiluminescence produced from the remaining probe in a luminometer. See, e.g., U.S. Pat. No. 5,283,174 and Norman C. Nelson et al., Nonisotopic Probing, Blotting, and Sequencing, ch. 17 (Larry J. Kricka ed., 2d ed. 1995, each of which is herein incorporated by reference in its entirety).

Another illustrative detection method provides for quantitative evaluation of the amplification process in real-time. Evaluation of an amplification process in “real-time” involves determining the amount of amplicon in the reaction mixture either continuously or periodically during the amplification reaction, and using the determined values to calculate the amount of target sequence initially present in the sample. A variety of methods for determining the amount of initial target sequence present in a sample based on real-time amplification are well known in the art. These include methods disclosed in U.S. Pat. Nos. 6,303,305 and 6,541,205, each of which is herein incorporated by reference in its entirety. Another method for determining the quantity of target sequence initially present in a sample, but which is not based on a real-time amplification, is disclosed in U.S. Pat. No. 5,710,029, herein incorporated by reference in its entirety.

Amplification products may be detected in real-time through the use of various self-hybridizing probes, most of which have a stem-loop structure. Such self-hybridizing probes are labeled so that they emit differently detectable signals, depending on whether the probes are in a self-hybridized state or an altered state through hybridization to a target sequence. By way of non-limiting example, “molecular torches” are a type of self-hybridizing probe that includes distinct regions of self-complementarity (referred to as “the target binding domain” and “the target closing domain”) which are connected by a joining region (e.g., non-nucleotide linker) and which hybridize to each other under predetermined hybridization assay conditions. In a preferred embodiment, molecular torches contain single-stranded base regions in the target binding domain that are from 1 to about 20 bases in length and are accessible for hybridization to a target sequence present in an amplification reaction under strand displacement conditions. Under strand displacement conditions, hybridization of the two complementary regions, which may be fully or partially complementary, of the molecular torch is favored, except in the presence of the target sequence, which will bind to the single-stranded region present in the target binding domain and displace all or a portion of the target closing domain. The target binding domain and the target closing domain of a molecular torch include a detectable label or a pair of interacting labels (e.g., luminescent/quencher) positioned so that a different signal is produced when the molecular torch is self-hybridized than when the molecular torch is hybridized to the target sequence, thereby permitting detection of probe:target duplexes in a test sample in the presence of unhybridized molecular torches. Molecular torches and a variety of types of interacting label pairs are disclosed in U.S. Pat. No. 6,534,274, herein incorporated by reference in its entirety.

Another example of a detection probe having self-complementarity is a “molecular beacon.” Molecular beacons include nucleic acid molecules having a target complementary sequence, an affinity pair (or nucleic acid arms) holding the probe in a closed conformation in the absence of a target sequence present in an amplification reaction, and a label pair that interacts when the probe is in a closed conformation. Hybridization of the target sequence and the target complementary sequence separates the members of the affinity pair, thereby shifting the probe to an open conformation. The shift to the open conformation is detectable due to reduced interaction of the label pair, which may be, for example, a fluorophore and a quencher (e.g., DABCYL and EDANS). Molecular beacons are disclosed in U.S. Pat. Nos. 5,925,517 and 6,150,097, herein incorporated by reference in its entirety.

Other self-hybridizing probes are well known to those of ordinary skill in the art. By way of non-limiting example, probe binding pairs having interacting labels, such as those disclosed in U.S. Pat. No. 5,928,862 (herein incorporated by reference in its entirety) might be adapted for use in the present invention. Probe systems used to detect single nucleotide polymorphisms (SNPs) might also be utilized in the present invention. Additional detection systems include “molecular switches,” as disclosed in U.S. Publ. No. 20050042638, herein incorporated by reference in its entirety. Other probes, such as those comprising intercalating dyes and/or fluorochromes, are also useful for detection of amplification products in the present invention. See, e.g., U.S. Pat. No. 5,814,447 (herein incorporated by reference in its entirety).

ii. Detection of Variant JAK/STAT Pathway Proteins

In other embodiments, variant JAK/STAT pathway (e.g., JAK1, JAK3, STAT5B, IL2RG) polypeptides are detected. Any suitable method may be used to detect truncated or mutant JAK/STAT pathway (e.g., JAK1, JAK3, STAT5B, IL2RG) polypeptides. For example, detection paradigms that can be employed to this end include optical methods, electrochemical methods (voltametry and amperometry techniques), atomic force microscopy, and radio frequency methods, e.g., multipolar resonance spectroscopy. Illustrative of optical methods, in addition to microscopy, both confocal and non-confocal, are detection of fluorescence, luminescence, chemiluminescence, absorbance, reflectance, transmittance, and birefringence or refractive index (e.g., surface plasmon resonance, ellipsometry, a resonant mirror method, a grating coupler waveguide method or interferometry).

1. Antibody Binding

In some embodiments, antibodies (see below for antibody production) are used to determine if an individual contains an allele encoding a variant JAK/STAT pathway (e.g., JAK1, JAK3, STAT5B, IL2RG) polypeptide. In preferred embodiments, antibodies are utilized that discriminate between variant (i.e., truncated proteins); and wild-type proteins. In some embodiments, the antibodies are directed to the C-terminus of JAK/STAT pathway (e.g., JAK1, JAK3, STAT5B, IL2RG) proteins. Proteins that are recognized by the N-terminal, but not the C-terminal antibody are truncated. In some embodiments, quantitative immunoassays are used to determine the ratios of C-terminal to N-terminal antibody binding. In other embodiments, identification of variants of JAK/STAT pathway (e.g., JAK1, JAK3, STAT5B, IL2RG) polypeptides is accomplished through the use of antibodies that differentially bind to wild type or variant forms of JAK/STAT pathway (e.g., JAK1, JAK3, STAT5B, IL2RG) proteins. In some embodiments, the present invention provides antibodies specific for a particular JAK/STAT pathway variant. For example, in some embodiments, separate antibodies are provided that are only specific for one JAK/STAT pathway variant as described herein (e.g., JAK1 p.F636L, JAK1 p.G646C, JAK1 p. Y654F, JAK1 p.V658F, JAK1 p.S7031, JAK1 p.T901R, JAK3 p.ΔKNC563, JAK3 p.G662W, JAK3 p.P664T, JAK3 p.M511I, JAK3 p. A573V, JAK3 p.R657. JAK3 p.Y980, JAK3 p. Y981, and JAK3 p. S9891, STAT5B p.T628S, STAT5B p.N642H, STAT5B p.Y699, and STAT5B p.Y665H, IL2RG p.ΔGSM268, IL2RG p.Y325, and IL2RG p. K315E). In some embodiments, such separate antibodies are specific for the entire JAK/STAT pathway variant. In some embodiments, such separate antibodies are specific for a fragment of such a JAK/STAT pathway variant. In some embodiments, such separate antibodies specific for a JAK/STAT pathway variant will not bind the respective wild type equivalent JAK/STAT pathway variant. In some embodiments, such separate antibodies specific for a JAK/STAT pathway variant will not bind different JAK/STAT pathway variants.

Antibody binding is detected by techniques known in the art (e.g., radioimmunoassay, ELISA (enzyme-linked immunosorbant assay), “sandwich” immunoassays, immunoradiometric assays, gel diffusion precipitation reactions, immunodiffusion assays, in situ immunoassays (e.g., using colloidal gold, enzyme or radioisotope labels, for example), Western blots, precipitation reactions, agglutination assays (e.g., gel agglutination assays, hemagglutination assays, etc.), complement fixation assays, immunofluorescence assays, protein A assays, and immunoelectrophoresis assays, etc.

In one embodiment, antibody binding is detected by detecting a label on the primary antibody. In another embodiment, the primary antibody is detected by detecting binding of a secondary antibody or reagent to the primary antibody. In a further embodiment, the secondary antibody is labeled. Many methods are known in the art for detecting binding in an immunoassay and are within the scope of the present invention.

In some embodiments, an automated detection assay is utilized. Methods for the automation of immunoassays include those described in U.S. Pat. Nos. 5,885,530, 4,981,785, 6,159,750, and 5,358,691, each of which is herein incorporated by reference. In some embodiments, the analysis and presentation of results is also automated. For example, in some embodiments, software that generates a prognosis based on the result of the immunoassay is utilized. In other embodiments, the immunoassay described in U.S. Pat. Nos. 5,599,677 and 5,672,480; each of which is herein incorporated by reference.

C. Kits for Detecting JAK/STAT Pathway Mutant or Variant Alleles

The present invention also provides kits for determining whether an individual contains a JAK/STAT pathway (e.g., JAK1, JAK3, STAT5B, IL2RG) wild-type or variant (e.g., mutant or polymorphic) allele. In some embodiments, the kits are useful for determining whether the subject has a mature T-cell leukemia (e.g., T-PLL, SS, MF) or to provide a prognosis to an individual diagnosed with a mature T-cell leukemia (e.g., T-PLL, SS, MF). The diagnostic kits are produced in a variety of ways. In some embodiments, the kits contain at least one reagent useful, necessary, or sufficient for specifically detecting a mutant or variant JAK/STAT pathway (e.g., JAK1, JAK3, STAT5B, IL2RG) allele or protein. In some embodiments, the kits contain reagents for detecting a truncation in a JAK/STAT pathway (e.g., JAK1, JAK3, STAT5B, IL2RG) polypeptide. In preferred embodiments, the reagent is a nucleic acid that hybridizes to nucleic acids containing the mutation and that does not bind to nucleic acids that do not contain the mutation. In other embodiments, the reagents are primers for amplifying the region of DNA containing the mutation. In still other embodiments, the reagents are antibodies that preferentially bind either the wild-type or truncated or variant JAK/STAT pathway (e.g., JAK1, JAK3, STAT5B, IL2RG) proteins.

In some embodiments, the kits include ancillary reagents such as buffering agents, nucleic acid stabilizing reagents, protein stabilizing reagents, and signal producing systems (e.g., florescence generating systems as Fret systems), and software (e.g., data analysis software). The test kit may be packages in any suitable manner, typically with the elements in a single container or various containers as necessary along with a sheet of instructions for carrying out the test. In some embodiments, the kits also preferably include a positive control sample.

In some embodiments, markers (e.g., those described herein) are detected alone or in combination with other markers in a panel or multiplex format. For example, in some embodiments, a plurality of markers are simultaneously detected in an array or multiplex format (e.g., using the detection methods described herein).

D. Bioinformatics

In some embodiments, a computer-based analysis program is used to translate raw data generated by detection assay (e.g., the presence, absence, or amount of a given JAK/STAT pathway (e.g., JAK1, JAK3, STAT5B, IL2RG) related allele or polypeptide) of the present invention into data of predictive value for a clinician. The clinician can access the predictive data using any suitable means. Thus, in some preferred embodiments, the present invention provides the further benefit that the clinician, who may not be trained in genetics or molecular biology, need not understand the raw data. The data is presented directly to the clinician in its most useful form. The clinician is then able to immediately utilize the information in order to optimize the care of the subject.

The present invention contemplates any method capable of receiving, processing, and transmitting the information to and from laboratories conducting the assays, information providers, medical personal, and subjects. For example, in some embodiments of the present invention, a sample (e.g., a biopsy or a blood or serum sample) is obtained from a subject and submitted to a profiling service (e.g., clinical lab at a medical facility, genomic profiling business, etc.), located in any part of the world (e.g., in a country different than the country where the subject resides or where the information is ultimately used) to generate raw data. Where the sample comprises a tissue or other biological sample, the subject may visit a medical center to have the sample obtained and sent to the profiling center, or subjects may collect the sample themselves (e.g., a urine sample) and directly send it to a profiling center. Where the sample comprises previously determined biological information, the information may be directly sent to the profiling service by the subject (e.g., an information card containing the information may be scanned by a computer and the data transmitted to a computer of the profiling center using an electronic communication systems). Once received by the profiling service, the sample is processed and a profile is produced (i.e., presence of wild type or mutant JAK/STAT pathway (e.g., JAK1, JAK3, STAT5B, IL2RG) related allele or protein), specific for the screening, diagnostic or prognostic information desired for the subject.

The profile data is then prepared in a format suitable for interpretation by a treating clinician. For example, rather than providing raw data, the prepared format may represent a diagnosis or risk assessment (e.g., diagnosis or prognosis of a mature T-cell leukemia) for the subject, along with recommendations for particular treatment options. The data may be displayed to the clinician by any suitable method. For example, in some embodiments, the profiling service generates a report that can be printed for the clinician (e.g., at the point of care) or displayed to the clinician on a computer monitor.

In some embodiments, the information is first analyzed at the point of care or at a regional facility. The raw data is then sent to a central processing facility for further analysis and/or to convert the raw data to information useful for a clinician or patient. The central processing facility provides the advantage of privacy (all data is stored in a central facility with uniform security protocols), speed, and uniformity of data analysis. The central processing facility can then control the fate of the data following treatment of the subject. For example, using an electronic communication system, the central facility can provide data to the clinician, the subject, or researchers.

In some embodiments, the subject is able to directly access the data using the electronic communication system. The subject may chose further intervention or counseling based on the results. In some embodiments, the data is used for research use. For example, the data may be used to further optimize the inclusion or elimination of markers as useful indicators of a particular condition or stage of disease.

In some embodiments, the methods disclosed herein are useful in monitoring the treatment of a mature T-cell leukemia (e.g., T-PLL, SS, MF). For example, in some embodiments, the methods may be performed immediately before, during and/or after a treatment to monitor treatment success. In some embodiments, the methods are performed at intervals on disease free patients to ensure treatment success.

The present invention also provides a variety of computer-related embodiments. Specifically, in some embodiments the invention provides computer programming for analyzing and comparing a pattern of a mature T-cell leukemia-specific marker detection results in a sample obtained from a subject to, for example, a library of such marker patterns known to be indicative of the presence or absence of a mature T-cell leukemia, or a particular stage or prognosis of a mature T-cell leukemia.

In some embodiments, the present invention provides computer programming for analyzing and comparing a first and a second pattern of a mature T-cell leukemia-specific marker detection results from a sample taken at least two different time points. In some embodiments, the first pattern may be indicative of a pre-cancerous condition and/or low risk condition for a mature T-cell leukemia and/or progression from a pre-cancerous condition to a cancerous condition. In such embodiments, the comparing provides for monitoring of the progression of the condition from the first time point to the second time point.

In yet another embodiment, the invention provides computer programming for analyzing and comparing a pattern of mature T-cell leukemia-specific marker detection results from a sample to a library of mature T-cell leukemia-specific marker patterns known to be indicative of the presence or absence of a mature T-cell leukemia (e.g., T-PLL, SS, MF), wherein the comparing provides, for example, a differential diagnosis between an aggressively malignant mature T-cell leukemia and a less aggressive mature T-cell leukemia (e.g., the marker pattern provides for staging and/or grading of the cancerous condition).

The methods and systems described herein can be implemented in numerous ways. In one embodiment, the methods involve use of a communications infrastructure, for example the internet. Several embodiments of the invention are discussed below. It is also to be understood that the present invention may be implemented in various forms of hardware, software, firmware, processors, distributed servers (e.g., as used in cloud computing) or a combination thereof. The methods and systems described herein can be implemented as a combination of hardware and software. The software can be implemented as an application program tangibly embodied on a program storage device, or different portions of the software implemented in the user's computing environment (e.g., as an applet) and on the reviewer's computing environment, where the reviewer may be located at a remote site (e.g., at a service provider's facility).

For example, during or after data input by the user, portions of the data processing can be performed in the user-side computing environment. For example, the user-side computing environment can be programmed to provide for defined test codes to denote platform, carrier/diagnostic test, or both; processing of data using defined flags, and/or generation of flag configurations, where the responses are transmitted as processed or partially processed responses to the reviewer's computing environment in the form of test code and flag configurations for subsequent execution of one or more algorithms to provide a results and/or generate a report in the reviewer's computing environment.

The application program for executing the algorithms described herein may be uploaded to, and executed by, a machine comprising any suitable architecture. In general, the machine involves a computer platform having hardware such as one or more central processing units (CPU), a random access memory (RAM), and input/output (I/O) interface(s). The computer platform also includes an operating system and microinstruction code. The various processes and functions described herein may either be part of the microinstruction code or part of the application program (or a combination thereof) which is executed via the operating system. In addition, various other peripheral devices may be connected to the computer platform such as an additional data storage device and a printing device.

As a computer system, the system generally includes a processor unit. The processor unit operates to receive information, which generally includes test data (e.g., specific gene products assayed), and test result data (e.g., the pattern of gastrointestinal neoplasm-specific marker detection results from a sample). This information received can be stored at least temporarily in a database, and data analyzed in comparison to a library of marker patterns known to be indicative of the presence or absence of a pre-cancerous condition, or known to be indicative of a stage and/or grade of gastrointestinal cancer.

Part or all of the input and output data can also be sent electronically; certain output data (e.g., reports) can be sent electronically or telephonically (e.g., by facsimile, e.g., using devices such as fax back). Exemplary output receiving devices can include a display element, a printer, a facsimile device and the like. Electronic forms of transmission and/or display can include email, interactive television, and the like. In some embodiments, all or a portion of the input data and/or all or a portion of the output data (e.g., usually at least the library of the pattern of gastrointestinal neoplasm-specific marker detection results known to be indicative of the presence or absence of a pre-cancerous condition) are maintained on a server for access, e.g., confidential access. The results may be accessed or sent to professionals as desired.

A system for use in the methods described herein generally includes at least one computer processor (e.g., where the method is carried out in its entirety at a single site) or at least two networked computer processors (e.g., where detected marker data for a sample obtained from a subject is to be input by a user (e.g., a technician or someone performing the assays)) and transmitted to a remote site to a second computer processor for analysis (e.g., where the pattern of mature T-cell leukemia-specific marker) detection results is compared to a library of patterns known to be indicative of the presence or absence of a pre-cancerous condition), where the first and second computer processors are connected by a network, e.g., via an intranet or internet). The system can also include a user component(s) for input; and a reviewer component(s) for review of data, and generation of reports, including detection of a pre-cancerous condition, staging and/or grading of a mature T-cell leukemia, or monitoring the progression of a pre-cancerous condition or mature T-cell leukemia. Additional components of the system can include a server component(s); and a database(s) for storing data (e.g., as in a database of report elements, e.g., a library of marker patterns known to be indicative of the presence or absence of a pre-cancerous condition and/or known to be indicative of a grade and/or a stage of a mature T-cell leukemia, or a relational database (RDB) which can include data input by the user and data output. The computer processors can be processors that are typically found in personal desktop computers (e.g., IBM, Dell, Macintosh), portable computers, mainframes, minicomputers, tablet computer, smart phone, or other computing devices.

The input components can be complete, stand-alone personal computers offering a full range of power and features to run applications. The user component usually operates under any desired operating system and includes a communication element (e.g., a modem or other hardware for connecting to a network using a cellular phone network, Wi-Fi, Bluetooth, Ethernet, etc.), one or more input devices (e.g., a keyboard, mouse, keypad, or other device used to transfer information or commands), a storage element (e.g., a hard drive or other computer-readable, computer-writable storage medium), and a display element (e.g., a monitor, television, LCD, LED, or other display device that conveys information to the user). The user enters input commands into the computer processor through an input device. Generally, the user interface is a graphical user interface (GUI) written for web browser applications.

The server component(s) can be a personal computer, a minicomputer, or a mainframe, or distributed across multiple servers (e.g., as in cloud computing applications) and offers data management, information sharing between clients, network administration and security. The application and any databases used can be on the same or different servers. Other computing arrangements for the user and server(s), including processing on a single machine such as a mainframe, a collection of machines, or other suitable configuration are contemplated. In general, the user and server machines work together to accomplish the processing of the present invention.

Where used, the database(s) is usually connected to the database server component and can be any device which will hold data. For example, the database can be any magnetic or optical storing device for a computer (e.g., CDROM, internal hard drive, tape drive). The database can be located remote to the server component (with access via a network, modem, etc.) or locally to the server component.

Where used in the system and methods, the database can be a relational database that is organized and accessed according to relationships between data items. The relational database is generally composed of a plurality of tables (entities). The rows of a table represent records (collections of information about separate items) and the columns represent fields (particular attributes of a record). In its simplest conception, the relational database is a collection of data entries that “relate” to each other through at least one common field.

Additional workstations equipped with computers and printers may be used at point of service to enter data and, in some embodiments, generate appropriate reports, if desired. The computer(s) can have a shortcut (e.g., on the desktop) to launch the application to facilitate initiation of data entry, transmission, analysis, report receipt, etc. as desired.

In certain embodiments, the present invention provides methods for obtaining a subject's risk profile for developing a mature T-cell leukemia or having an aggressive form of a mature T-cell leukemia. In some embodiments, such methods involve obtaining a blood or blood product sample from a subject (e.g., a human at risk for developing a mature T-cell leukemia; a human undergoing a routine physical examination, or a human diagnosed with a mature T-cell leukemia), detecting the presence or absence of JAK/STAT pathway variants described herein (e.g., JAK1, JAK3, STAT5B, IL2RG) in the sample, and generating a risk profile for developing a mature T-cell leukemia (e.g., T-PLL, SS, MF) or progressing to a metastatic or aggressive form of such mature T-cell leukemia. For example, in some embodiments, a generated profile will change depending upon specific markers and detected as present or absent or at defined threshold levels. The present invention is not limited to a particular manner of generating the risk profile. In some embodiments, a processor (e.g., computer) is used to generate such a risk profile. In some embodiments, the processor uses an algorithm (e.g., software) specific for interpreting the presence and absence of specific exfoliated epithelial markers as determined with the methods of the present invention. In some embodiments, the presence and absence of specific JAK/STAT pathway variants described herein (e.g., JAK1, JAK3, STAT5B, IL2RG) as determined with the methods of the present invention are imputed into such an algorithm, and the risk profile is reported based upon a comparison of such input with established norms (e.g., established norm for pre-cancerous condition, established norm for various risk levels for developing a mature T-cell leukemia, established norm for subjects diagnosed with various stages of a mature T-cell leukemia). In some embodiments, the risk profile indicates a subject's risk for developing a mature T-cell leukemia or a subject's risk for re-developing a mature T-cell leukemia. In some embodiments, the risk profile indicates a subject to be, for example, a very low, a low, a moderate, a high, and a very high chance of developing or re-developing a mature T-cell leukemia or having a poor prognosis (e.g., likelihood of long term survival) from a mature T-cell leukemia. In some embodiments, a health care provider (e.g., an oncologist) will use such a risk profile in determining a course of treatment or intervention (e.g., biopsy, wait and see, referral to an oncologist, referral to a surgeon, etc.).

D. Mass Spectrometry

Mass spectrometry is a particularly powerful methodology to resolve different forms of a protein because the different forms typically have different masses that can be resolved by mass spectrometry. Accordingly, if one form of a protein is a superior biomarker for a disease than another form of the biomarker, mass spectrometry may be able to specifically detect and measure the useful form where traditional immunoassay fails to distinguish the forms and fails to specifically detect to useful biomarker.

One useful methodology for detecting a specific JAK/STAT pathway variant described herein (e.g., JAK1, JAK3, STAT5B, IL2RG) combines mass spectrometry with immunoassay. First, a biospecific capture reagent (e.g., an antibody, aptamer or Affibody that recognizes the biomarker and other forms of it) is used to capture the biomarker of interest (e.g., a JAK1 variant, a JAK3 variant, a STAT5B variant, a IL2RG variant). Preferably, the biospecific capture reagent is bound to a solid phase, such as a bead, a plate, a membrane or an array. After unbound materials are washed away, the captured analytes are detected and/or measured by mass spectrometry. In some embodiments, such methods also permit capture of protein interactors, if present, that are bound to the proteins or that are otherwise recognized by antibodies and that, themselves, can be biomarkers. Various forms of mass spectrometry are useful for detecting the protein forms, including laser desorption approaches, such as traditional MALDI or SELDI, and electrospray ionization.

In some embodiments, a biomarker of this invention (e.g., a JAK1 variant, a JAK3 variant, a STAT5B variant, a IL2RG variant) is detected by mass spectrometry, a method that employs a mass spectrometer to detect gas phase ions. Examples of mass spectrometers are time-of-flight, magnetic sector, quadrupole filter, ion trap, ion cyclotron resonance, electrostatic sector analyzer and hybrids of these.

In some embodiments, the mass spectrometer is a laser desorption/ionization mass spectrometer. In laser desorption/ionization mass spectrometry, the analytes are placed on the surface of a mass spectrometry probe, a device adapted to engage a probe interface of the mass spectrometer and to present an analyte to ionizing energy for ionization and introduction into a mass spectrometer. A laser desorption mass spectrometer employs laser energy, typically from an ultraviolet laser, but also from an infrared laser, to desorb analytes from a surface, to volatilize and ionize them and make them available to the ion optics of the mass spectrometer. (e.g., a JAK1 variant, a JAK3 variant, a STAT5B variant, a IL2RG variant)

In some embodiments, the mass spectrometric technique for use is “Surface Enhanced Laser Desorption and Ionization” or “SELDI,” as described, for example, in U.S. Pat. No. 5,719,060 and No. 6,225,047; each herein incorporated by reference in its entirety. This refers to a method of desorption/ionization gas phase ion spectrometry (e.g. mass spectrometry) in which an analyte (e.g., one or more of the biomarkers of the present invention) is captured on the surface of a SELDI mass spectrometry probe. There are several versions of SELDI.

One version of SELDI is called “affinity capture mass spectrometry.” It also is called “Surface-Enhanced Affinity Capture” or “SEAC”. This version involves the use of probes that have a material on the probe surface that captures analytes through a non-covalent affinity interaction (adsorption) between the material and the analyte. The material is variously called an “adsorbent,” a “capture reagent,” an “affinity reagent” or a “binding moiety.” Such probes can be referred to as “affinity capture probes” and as having an “adsorbent surface.” The capture reagent can be any material capable of binding an analyte. The capture reagent is attached to the probe surface by physisorption or chemisorption. In certain embodiments the probes have the capture reagent already attached to the surface. In other embodiments, the probes are pre-activated and include a reactive moiety that is capable of binding the capture reagent, e.g., through a reaction forming a covalent or coordinate covalent bond. Epoxide and acyl-imidizole are useful reactive moieties to covalently bind polypeptide capture reagents such as antibodies or cellular receptors. Nitrilotriacetic acid and iminodiacetic acid are useful reactive moieties that function as chelating agents to bind metal ions that interact non-covalently with histidine containing peptides. Adsorbents are generally classified as chromatographic adsorbents and biospecific adsorbents.

“Chromatographic adsorbent” refers to an adsorbent material typically used in chromatography. Chromatographic adsorbents include, for example, ion exchange materials, metal chelators (e.g., nitrilotriacetic acid or iminodiacetic acid), immobilized metal chelates, hydrophobic interaction adsorbents, hydrophilic interaction adsorbents, dyes, simple biomolecules (e.g., nucleotides, amino acids, simple sugars and fatty acids) and mixed mode adsorbents (e.g., hydrophobic attraction/electrostatic repulsion adsorbents).

“Biospecific adsorbent” refers to an adsorbent comprising a biomolecule, e.g., a nucleic acid molecule (e.g., an aptamer), a polypeptide, a polysaccharide, a lipid, a steroid or a conjugate of these (e.g., a glycoprotein, a lipoprotein, a glycolipid, a nucleic acid (e.g., DNA)-protein conjugate). In certain instances, the biospecific adsorbent can be a macromolecular structure such as a multiprotein complex, a biological membrane or a virus. Examples of biospecific adsorbents are antibodies, receptor proteins and nucleic acids. Biospecific adsorbents typically have higher specificity for a target analyte than chromatographic adsorbents. Further examples of adsorbents for use in SELDI can be found in U.S. Pat. No. 6,225,047; herein incorporated by reference in its entirety. A “bioselective adsorbent” refers to an adsorbent that binds to an analyte with an affinity of at least 10⁻⁸M.

Protein biochips produced by Ciphergen Biosystems, Inc. comprise surfaces having chromatographic or biospecific adsorbents attached thereto at addressable locations. Ciphergen ProteinChip® arrays include NP20 (hydrophilic); H4 and HSO (hydrophobic); SAX-2, Q-10 and LSAX-30 (anion exchange); WCX-2, CM-10 and LWCX-30 (cation exchange); IMAC-3, MAC-30 and IMAC 40 (metal chelate); and PS-10, PS-20 (reactive surface with acyl-imidizole, epoxide) and PG-20 (protein G coupled through acyl-imidizole). Hydrophobic ProteinChip arrays have isopropyl or nonylphenoxy-poly(ethylene glycol)methacrylate functionalities. Anion exchange ProteinChip arrays have quaternary ammonium functionalities. Cation exchange ProteinChip arrays have carboxylate functionalities. Immobilized metal chelate ProteinChip arrays have nitrilotriacetic acid functionalities that adsorb transition metal ions, such as copper, nickel, zinc, and gallium, by chelation. Preactivated ProteinChip arrays have acyl-imidizole or epoxide functional groups that can react with groups on proteins for covalent binding. Such biochips are further described in: U.S. Pat. Nos. 7,045,366, 6,579,719; 6,897,072; 6,555,813; U.S. Patent Publication Nos. U.S. 2003-0032043; US 2003-0218130; and PCT International Publication No. WO 03/040700; each herein incorporated by reference in its entirety.

In general, a probe with an adsorbent surface is contacted with the sample for a period of time sufficient to allow the biomarker or biomarkers that may be present in the sample to bind to the adsorbent. After an incubation period, the substrate is washed to remove unbound material. Any suitable washing solutions can be used; preferably, aqueous solutions are employed. The extent to which molecules remain bound can be manipulated by adjusting the stringency of the wash. The elution characteristics of a wash solution can depend, for example, on pH, ionic strength, hydrophobicity, degree of chaotropism, detergent strength, and temperature.

The biomarkers bound to the substrates are detected in a gas phase ion spectrometer such as a time-of-flight mass spectrometer. The biomarkers are ionized by an ionization source such as a laser, the generated ions are collected by an ion optic assembly, and then a mass analyzer disperses and analyzes the passing ions. The detector then translates information of the detected ions into mass-to-charge ratios. Detection of a biomarker typically will involve detection of signal intensity. Thus, both the quantity and mass of the biomarker can be determined.

Another version of SELDI is Surface-Enhanced Neat Desorption (SEND), which involves the use of probes comprising energy absorbing molecules that are chemically bound to the probe surface (“SEND probe”). The phrase “energy absorbing molecules” (EAM) denotes molecules that are capable of absorbing energy from a laser desorption/ionization source and, thereafter, contribute to desorption and ionization of analyte molecules in contact therewith. The EAM category includes molecules used in MALDI, frequently referred to as “matrix,” and is exemplified by cinnamic acid derivatives, sinapinic acid (SPA), cyano-hydroxy-cinnamic acid (CHCA) and dihydroxybenzoic acid, ferulic acid, and hydroxyacetophenone derivatives. In certain embodiments, the energy absorbing molecule is incorporated into a linear or cross-linked polymer, e.g., a polymethacrylate. For example, the composition can be a co-polymer of α-cyano-4-methacryloyloxycinnamic acid and acrylate. In another embodiment, the composition is a co-polymer of α-cyano-4-methacryloyloxycinnamic acid, acrylate and 3-(tri-ethoxy)silyl propyl methacrylate. In another embodiment, the composition is a co-polymer of α-cyano-4-methacryloyloxycinnamic acid and octadecylmethacrylate “C18 SEND”). SEND is further described in U.S. Pat. No. 6,124,137 and PCT International Publication No. WO 03/64594; each herein incorporated in its entirety.

SEAC/SEND is a version of SELDI in which both a capture reagent and an energy absorbing molecule are attached to the sample presenting surface. SEAC/SEND probes therefore allow the capture of analytes through affinity capture and ionization/desorption without the need to apply external matrix. The C18 SEND biochip is a version of SEAC/SEND, comprising a C18 moiety which functions as a capture reagent, and a CHCA moiety which functions as an energy absorbing moiety.

E. Biochip Detection

In some embodiments, a sample is analyzed by means of a biochip. Biochips generally comprise solid substrates and have a generally planar surface, to which a capture reagent (also called an adsorbent or affinity reagent) is attached. Frequently, the surface of a biochip comprises a plurality of addressable locations, each of which has the capture reagent bound there. For example, in some embodiments, the present invention provides biochips having attached thereon one or more capture reagents specific for a JAK/STAT variant of the present invention (e.g., a JAK1 variant, a JAK3 variant, a STAT5B variant, a IL2RG variant).

Protein biochips are biochips adapted for the capture of polypeptides (e.g., a JAK/STAT variant of the present invention (e.g., a JAK1 variant, a JAK3 variant, a STAT5B variant, a IL2RG variant)). Many protein biochips are described in the art. These include, for example, protein biochips produced by Ciphergen Biosystems, Inc. (Fremont, Calif.), Zyomyx (Hayward, Calif.), Invitrogen (Carlsbad, Calif.), Biacore (Uppsala, Sweden) and Procognia (Berkshire, UK). Examples of such protein biochips are described in the following patents or published patent applications: U.S. Pat. Nos. 6,225,047, 6,537,749, 6,329,209, and 5,242,828, and PCT International Publication Nos. WO 00/56934, and WO 03/048768; each herein incorporated by reference in its entirety.

### II. Therapeutic Management

In certain embodiments, the present invention provides methods for managing a subject's treatment based on the status (e.g., presence or absence of mature T-cell leukemia). Such management includes the actions of the physician or clinician subsequent to determining mature T-cell leukemia status. For example, if a physician makes a diagnosis of a mature T-cell leukemia, then a certain regime of treatment, such as prescription or administration of therapeutic agent might follow. Alternatively, a diagnosis of non-mature T-cell leukemia might be followed with further testing to determine a specific disease that the patient might be suffering from. Also, if the diagnostic test gives an inconclusive result on mature T-cell leukemia status, further tests may be called for.

### III. Determining Therapeutic Efficacy of Pharmaceutical Drug

In another embodiment, the present invention provides methods for determining the therapeutic efficacy of a pharmaceutical drug (e.g., a pharmaceutical drug for treating mature T-cell leukemia). These methods are useful in performing clinical trials of the drug, as well as monitoring the progress of a patient on the drug. Therapy or clinical trials involve administering the drug in a particular regimen. The regimen may involve a single dose of the drug or multiple doses of the drug over time. The doctor or clinical researcher monitors the effect of the drug on the patient or subject over the course of administration. If the drug has a pharmacological impact on the condition, the amounts or relative amounts (e.g., the pattern or profile) of the biomarkers (e.g., any of the variant forms of JAK1, JAK3, STAT5B, IL2RG described herein) of this invention changes toward a non-disease profile. Therefore, one can follow the course of the amounts of these biomarkers in the subject during the course of treatment. Accordingly, this method involves measuring one or more biomarkers in a subject receiving drug therapy, and correlating the amounts of the biomarkers with the disease status of the subject. One embodiment of this method involves determining the levels of the biomarkers at least two different time points during a course of drug therapy, e.g., a first time and a second time, and comparing the change in amounts of the biomarkers, if any. For example, the biomarkers can be measured before and after drug administration or at two different time points during drug administration. The effect of therapy is determined based on these comparisons. If a treatment is effective, then the biomarkers will trend toward normal, while if treatment is ineffective, the biomarkers will trend toward disease indications. If a treatment is effective, then the biomarkers will trend toward normal, while if treatment is ineffective, the biomarkers will trend toward disease indications.

### IV. Compositions of Matter

In another aspect, the present invention provides compositions of matter based on the biomarkers of this invention. For example, in one embodiment, the present invention provides a biomarker of this invention in purified form. Purified biomarkers have utility as antigens to raise antibodies. Purified biomarkers also have utility as standards in assay procedures. As used herein, a “purified biomarker” is a biomarker that has been isolated from other proteins and peptides, and/or other material from the biological sample in which the biomarker is found. For example, in some embodiments, the present invention provides compositions comprising a purified JAK/STAT variant (e.g., any of the JAK1 variants described herein, JAK3 variants described herein, STAT5B variants described herein, IL2RG variants described herein).

Biomarkers may be purified using any method known in the art, including, but not limited to, mechanical separation (e.g., centrifugation), ammonium sulphate precipitation, dialysis (including size-exclusion dialysis), size-exclusion chromatography, affinity chromatography, anion-exchange chromatography, cation-exchange chromatography, and methal-chelate chromatography. Such methods may be performed at any appropriate scale, for example, in a chromatography column, or on a biochip.

In another embodiment, the present invention provides a biospecific capture reagent, optionally in purified form, that specifically binds a biomarker of this invention. In one embodiment, the biospecific capture reagent is an antibody. Such compositions are useful for detecting the biomarker in a detection assay, e.g., for diagnostics.

In another embodiment, this invention provides an article comprising a biospecific capture reagent that binds a biomarker of this invention, wherein the reagent is bound to a solid phase. For example, this invention contemplates a device comprising bead, chip, membrane, monolith or microtiter plate derivatized with the biospecific capture reagent. Such articles are useful in biomarker detection assays.

In another aspect the present invention provides a composition comprising a biospecific capture reagent, such as an antibody, bound to a biomarker of this invention, the composition optionally being in purified form. Such compositions are useful for purifying the biomarker or in assays for detecting the biomarker.

In another embodiment, this invention provides an article comprising a solid substrate to which is attached an adsorbent, e.g., a chromatographic adsorbent or a biospecific capture reagent, to which is further bound a biomarker of this invention.

In another embodiment, the invention provides compositions comprising reaction mixtures formed through, for example, binding of a biomarker of the present invention with a detection marker (e.g., antibody, proble, biochip, etc.) (e.g., via a detection assay of the present invention). In some embodiments, “reaction mixture” comprises any material sufficient, necessary, or useful for conducting any of the assays described herein. In some embodiments, the present invention provides compositions comprising reaction mixtures comprising extension products complementary to a specific mutation. In some embodiments, the present invention provides compositions comprising reaction mixtures comprising extension products complementary to a specific mutation and sequences immediately surrounding such a mutation. In some embodiments, the extension product has thereon an labeling agent (e.g., a fluorophore or other lable). In some embodiments, the present invention provides compositions comprising reaction mixtures comprising extension products complementary to a specific mutation bound with such a complementary sequence. In some embodiments, the present invention provides compositions comprising reaction mixtures comprising extension products complementary to a specific mutation bound with such a complementary sequence, wherein the binding is to a solid surface, a biochip (e.g., in single copy or multiple copies). In some embodiments, the present invention provides compositions comprising fragments of a peptide of interest. In some embodiments, the present invention provides compositions comprising a peptide of interest in a mass-spectrometry compatible buffer.

### V. Kits for Detection of Mature T-cell Leukemia Biomarkers

In another aspect, the present invention provides kits for qualifying mature T-cell leukemia status, which kits are used to detect one or more of the biomarkers according to the invention. In one embodiment, the kit comprises a solid support, such as a chip, a microtiter plate or a bead or resin having a capture reagent attached thereon, wherein the capture reagent binds a biomarker of the invention. Thus, for example, the kits of the present invention can comprise mass spectrometry probes for SELDI, such as ProteinChip® arrays. In the case of biospecfic capture reagents, the kit can comprise a solid support with a reactive surface, and a container comprising the biospecific capture reagent.

In some embodiments, the kit can also comprise a washing solution or instructions for making a washing solution, in which the combination of the capture reagent and the washing solution allows capture of the biomarker or biomarkers on the solid support for subsequent detection by, e.g., mass spectrometry. The kit may include more than type of adsorbent, each present on a different solid support.

In some embodiments, such a kit can comprise instructions for suitable operational parameters in the form of a label or separate insert. For example, the instructions may inform a consumer about how to collect the sample, how to wash the probe or the particular biomarkers to be detected.

In some embodiments, the kit can comprise one or more containers with biomarker samples, to be used as standard(s) for calibration.

In some embodiments, the kit can comprise software necessary for interpreting results and/or generating prospective therapeutic outcomes. In some embodiments, for example, the software provides mutation specific databases and comparison programs.

### VI. Additional Embodiments

The following additional embodiments are contemplated.

1. A method for detecting one or more JAK/STAT pathway variants associated with a mature T-cell leukemia in a subject, comprising:

a) contacting a sample from a subject with a JAK/STAT pathway variant detection assay under conditions that the presence of a JAK/STAT pathway variant associated with a mature T-cell leukemia is determined; and

b) diagnosing said subject with a mature T-cell leukemia when one or more of said JAK/STAT pathway variants are present in said sample.

2. The method of claim 1, wherein said one or more JAK/STAT pathway variants encodes a loss of function mutation and/or a gain of function mutation.
3. The method of claim 1, wherein said subject is a human patient.
4. The method of claim 1, wherein said JAK/STAT pathway variant is one or more variants selected from JAK1, JAK3, STAT5B, and IL2RG.
5. The method of claim 4, wherein said JAK1 variant is one or more JAK1 mutations selected from the group consisting of JAK1 p.F636L, JAK1 p.G646C, JAK1 p. Y654F, JAK1 p.V658F, JAK1 p.S703I, and JAK1 p.T901R.
6. The method of claim 4, wherein said JAK3 variant is one or more JAK3 mutations selected from the group consisting of JAK3 p.M511I, JAK3 p.ΔKNC563, JAK3 p. A573V, JAK3 p.R657, JAK3 p.G662W, JAK3 p.P664T, JAK3 p.Y980, JAK3 p. Y981, and JAK3 p.S989I.
7. The method of claim 4, wherein said STAT5B variant is one or more STAT5B mutations selected from the group consisting of STAT5B p.T628S, STAT5B p.R659c, STAT5B p.Q706L, STAT5B p.N642H, STAT5B p.Y699, and STAT5B p.Y665H.
8. The method of claim 4, wherein said IL2RG variant is one or more IL2RG mutations selected from the group consisting of IL2RG p.ΔGSM268, IL2RG p.Y325, and IL2RG p. K315E.
9. The method of claim 1, wherein said mature T-cell leukemia is T-cell prolymphocytic leukemia.

10. The method of claim 9, wherein one or more JAK/STAT pathway variants are selected from the group consisting of JAK1 p.V658F, JAK1 p.S703I, JAK1 p.T901R, JAK3 p.ΔKNC563, JAK3 p.M511I, JAK3 p. A573V, JAK3 p.R657, STAT5B p.R659c, STAT5B p.Q706L, STAT5B p.T628S, STAT5B p.R659c, STAT5B p.Q706L, STAT5B p.N642H, STAT5B p.Y665H, IL2RG p.ΔGSM268, and IL2RG p. K315E.

11. The method of claim 1, wherein said mature T-cell leukemia is Sezary syndrome.
12. The method of claim 11, wherein one or more JAK/STAT pathway variants are selected from the group consisting of JAK1 p. Y654F, JAK3 p. A573V, JAK3 p.Y980, JAK3 p. Y981, JAK3 p.S989I, STAT5B p.Y699, STAT5B p.N642H, and I12RG p.Y325.
13. The method of claim 1, wherein said determining comprises detecting variant JAK1, JAK3, STAT5B, and IL2RG nucleic acids or polypeptides.
14. The method of claim 13, wherein said detecting variant JAK1, JAK3, STAT5B, and IL2RG nucleic acids comprises one or more nucleic acid detection method selected from the group consisting of sequencing, amplification and hybridization.
15. The method of claim 1, wherein said biological sample is selected from the group consisting of a tissue sample, a cell sample, and a blood sample.
16. The method of claim 1, wherein said determining comprises a computer implemented method.
17. The method of claim 16, wherein said computer implemented method comprises analyzing JAK1, JAK3, STAT5B, and IL2RG variant information and displaying said information to a user.
18. The method of claim 1, further comprising the step of treating said subject for a mature T-cell leukemia and monitoring said subject for the presence of JAK1, JAK3, STAT5B, and IL2RG variants associated with said mature T-cell leukemia.
19. The method of claim 1, further comprising the step of treating said subject for a mature T-cell leukemia under condition such that at least one symptom of said mature T-cell leukemia is diminished or eliminated.
20. The method of claim 19, wherein said treating comprises inhibiting JAK1, JAK3, STAT5B, and/or IL2RG expression and/or activity.
21. The method of claim 20, wherein said inhibiting STAT5B expression and/or activity is accomplished through administration of an agent configured to inhibit STAT5B expression pimozide.
22. The method of claim, wherein said inhibiting JAK1 and/or JAK3 expression and/or activity is accomplished through administration of an agent configured to inhibit JAK1 and/or JAK3 expression and/or activity, wherein said agent is selected from the group consisting of ruxolitinib, tofacitinib, baricitinib, CYT387, and lestaurtinib.
23. The method of claim 21, further comprising administering one or more agents for treating a mature T-cell leukemia.
24. The method of claim 23, wherein said one or more agents is selected from the group consisting of a purine analog (e.g., pentostatin, fludarabine, cladrbine), chlorambucil, cyclophosphamide, doxorubicin, vincristine, prednisone (CHOP), cyclophosphamide, vincristine, prednisone (COP), and vincristine, doxorubicin, prednisone, etoposide, cyclophosphamide, bleomycin, alemtuzumab, and vorinostat.
25. The method of claim 1, further comprising the step of detecting a variant in one or more additional genes associated with a mature T-cell leukemia.
26. The method of claim 25, wherein said one or more genes are selected from the group consisting of CHEK2, EZH2, and FBXW10.
27. Use of a variant JAK/STAT pathway nucleic acid or polypeptide for detecting a mature T-cell leukemia in a subject.
28. The use of claim 27, wherein said JAK/STAT pathway variant encodes a loss of function mutation and/or a gain of function mutuation.
29. The use of claim 27, wherein said subject is a human subject.
30. The use of claim 27, wherein said JAK/STAT pathway variant is one or more variants selected from JAK1, JAK3, STAT5B, and IL2RG.
31. The use of claim 30, wherein said JAK1 variant is one or more JAK1 mutations selected from the group consisting of JAK1 p.F636L, JAK1 p.G646C, JAK1 p. Y654F, JAK1 p.V658F, JAK1 p.S703I, and JAK1 p.T901R.
32. The use of claim 30, wherein said JAK3 variant is one or more JAK3 mutations selected from the group consisting of JAK3 p.M511I, JAK3 p.ΔKNC563, JAK3 p. A573V, JAK3 p.R657., JAK3 p.G662W, JAK3 p.P664T, JAK3 p.Y980, JAK3 p. Y981, and JAK3 p.S989I.
33. The use of claim 30, wherein said STAT5B variant is one or more STAT5B mutations selected from the group consisting of STAT5B p.T628S, STAT5B p.R659c, STAT5B p.Q706L, STAT5B p.N642H, STAT5B p.Y699, and STAT5B p.Y665H.
34. The use of claim 30, wherein said IL2RG variant is one or more IL2RG mutations selected from the group consisting of IL2RG p.ΔGSM268, IL2RG p.Y325 and IL2RG p. K315E.
35. The use of claim 27, wherein said mature T-cell leukemia is T-cell prolymphocytic leukemia.
36. The use of claim 35, wherein one or more JAK/STAT pathway variants are selected from the group consisting of JAK1 p.V658F, JAK1 p.S703I, JAK1 p.T901R, JAK3 p.ΔKNC563, JAK3 p.M511I, JAK3 p. A573V, JAK3 p.R657, STAT5B p.R659c, STAT5B p.Q706L, STAT5B p.T628S, STAT5B p.N642H, STAT5B p.Y665H, and IL2RG p.ΔGSM268, IL2RG p. K315E.
37. The use of claim 27, wherein said mature T-cell leukemia is Sezary syndrome.
38. The use of claim 37, wherein one or more JAK/STAT pathway variants are selected from the group consisting of JAK1 p. Y654F, JAK3 p. A573V, JAK3 p.Y980, JAK3 p. Y981, JAK3 p.S989I, STAT5B p.Y699, STAT5B p.N642H, and I12RG p.Y325.
39. The use of claim 27, wherein said determining comprises detecting variant JAK1, JAK3, STAT5B, and IL2RG nucleic acids or polypeptides.
40. The use of claim 39, wherein said detecting variant JAK1, JAK3, STAT5B, and IL2RG nucleic acids comprises one or more nucleic acid detection method selected from the group consisting of sequencing, amplification and hybridization.
41. A method of determining a decreased time to adverse outcome in a subject diagnosed with a mature T-cell leukemia, comprising:

a) contacting a sample from a subject with a JAK/STAT pathway variant detection assay under conditions that the presence of a JAK/STAT pathway variant associated with a mature T-cell leukemia is determined; and

c) detecting a decreased time to adverse outcome in said subject when said JAK/STAT pathway variants are present in said sample.

42. The method of claim 41, wherein said adverse outcome is selected from the group consisting of relapse of said mature T-cell leukemia, metastasis, or death.
43. The method of claim 41, wherein said JAK/STAT pathway variant encodes a loss of function mutation and/or a gain of function mutuation.
44. The method of claim 41, wherein said subject is a human subject.
45. The method of claim 41, wherein said JAK/STAT pathway variant is one or more variants selected from JAK1, JAK3, STAT5B, and IL2RG.

46. The method of claim 45, wherein said JAK1 variant is one or more JAK1 mutations selected from the group consisting of JAK1 p.F636L, JAK1 p.G646C, JAK1 p. Y654F, JAK1 p.V658F, JAK1 p.S703I, and JAK1 p.T901R.

47. The method of claim 45, wherein said JAK3 variant is one or more JAK3 mutations selected from the group consisting of JAK3 p.ΔKNC563, JAK3 p.M511I, JAK3 p. A573V, JAK3 p.R657, JAK3 p.G662W, JAK3 p.P664T, JAK3 p.Y980, JAK3 p. Y981, and JAK3 p.S989I.
48. The method of claim 45, wherein said STAT5B variant is one or more STAT5B mutations selected from the group consisting of STAT5B p.T628S, STAT5B p.R659c, STAT5B p.Q706L, STAT5B p.N642H, STAT5B p.Y699, and STAT5B p.Y665H.
49. The method of claim 45, wherein said IL2RG variant is one or more IL2RG mutations selected from the group consisting of IL2RG p.ΔGSM268, IL2RG p.Y325, and IL2RG p. K315E.
50. The method of claim 41, wherein said mature T-cell leukemia is T-cell prolymphocytic leukemia.
51. The method of claim 50, wherein one or more JAK/STAT pathway variants are selected from the group consisting of JAK1 p.V658F, JAK1 p.S703I, JAK1 p.T901R, JAK3 p.ΔKNC563, JAK3 p.M511I, JAK3 p. A573V, JAK3 p.R657, STAT5B p.T628S, STAT5B p.R659c, STAT5B p.Q706L, STAT5B p.N642H, STAT5B p.Y665H, IL2RG p.ΔGSM268, and IL2RG p. K315E.
52. The method of claim 41, wherein said mature T-cell leukemia is Sezary syndrome.
53. The method of claim 52, wherein one or more JAK/STAT pathway variants are selected from the group consisting of JAK1 p. Y654F, JAK3 p. A573V, JAK3 p.Y980, JAK3 p.Y981, JAK3 p.S989I, STAT5B p.Y699, STAT5B p.N642H, and I12RG p.Y325.
54. The method of claim 41, wherein said determining comprises detecting variant JAK1, JAK3, STAT5B, and IL2RG nucleic acids or polypeptides.

55. The method of claim 54, wherein said detecting variant JAK1, JAK3, STAT5B, and IL2RG nucleic acids comprises one or more nucleic acid detection method selected from the group consisting of sequencing, amplification and hybridization.

### EXPERIMENTAL

The following examples are provided in order to demonstrate and further illustrate certain preferred embodiments and aspects of the present invention and are not to be construed as limiting the scope thereof.

### Example I

This example demonstrates that constitutive activation of the JAK-STAT pathway could play a role in the pathogenesis of mature T-cell leukemia.

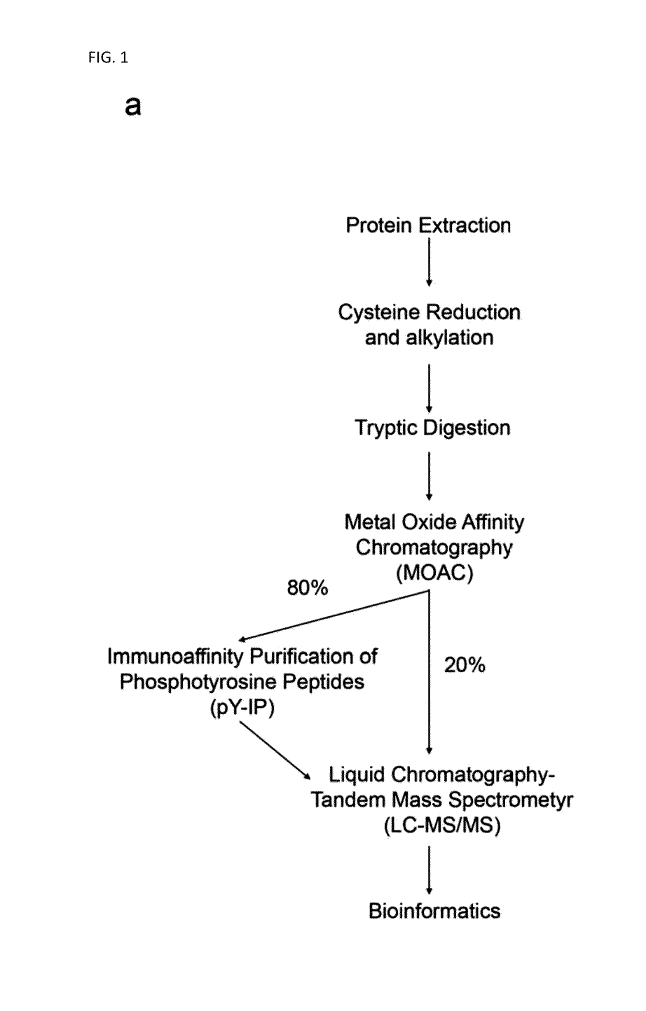

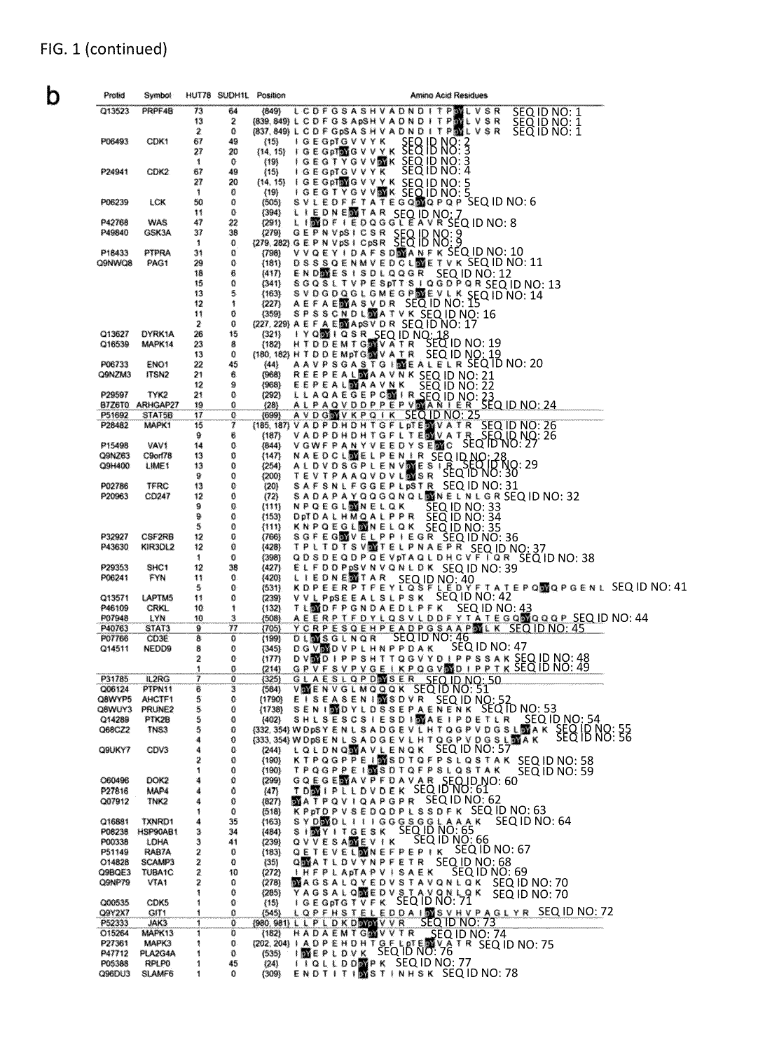

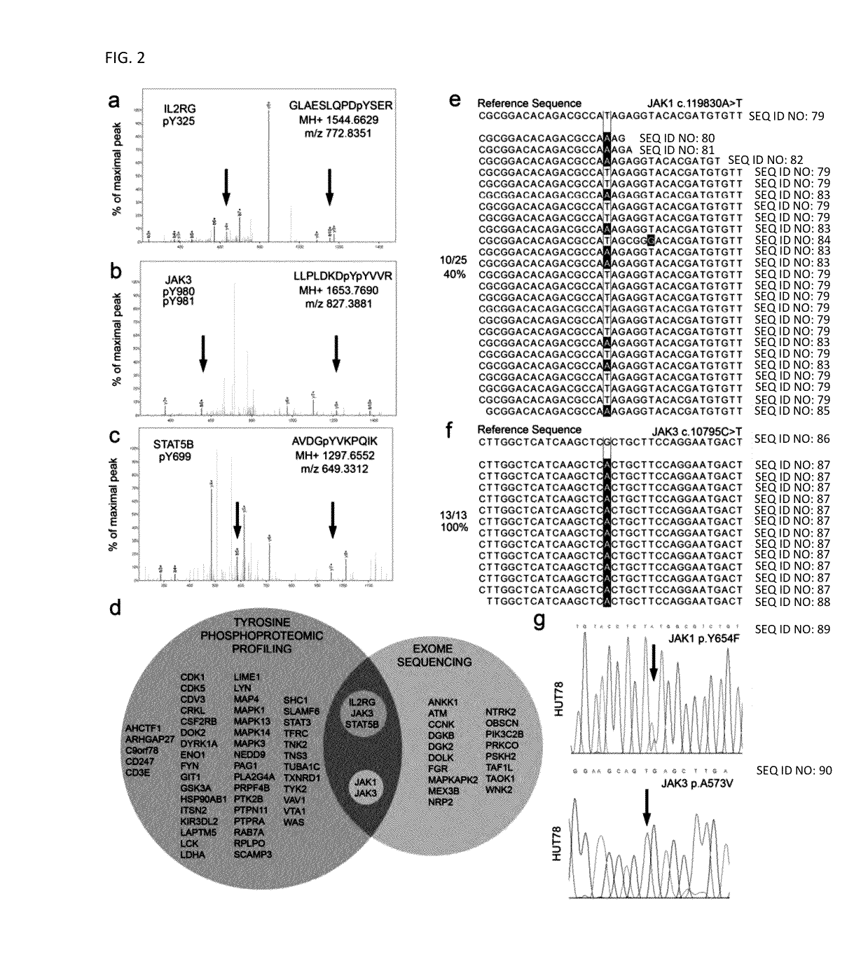

Unbiased tandem-mass spectrometry phosphoproteomic analysis (see, e.g., Walters, D. K. et al. 2005 Cancer Cell 10, 65-75; Rush, J. et al. 2006 Nat Biotechnol 23, 94-101; each herein incorporated by reference in its entirety) (FIG. 1) of the SS-derived mature T-cell leukemia cell line, HUT78, revealed numerous phosphorylated peptides corresponding to proteins whose phosphorylation has not previously been reported in T-cell leukemias. Among 59 proteins with tyrosine-phosphorylated peptides in HUT78 cells, the IL2RG p.Y325, JAK3 p.Y980/p.Y981 and the STAT5B p.Y699 residues were identified with high spectral counts suggesting constitutive activation of the JAK-STAT pathway (FIG. 2a-d). To identify possible mutational mechanisms underlying the constitutive JAK-STAT activation detected by phosphoproteomic screening, WES was performed on HUT78 cells (FIG. 2d, right). This analysis revealed mutations in both JAK1 (p.Y654F) and JAK3 (p.A573V; FIG. 2e-g). While the JAK3 p.A573V mutation had previously been reported to be associated with leukemic progression in acute lymphoblastic T-cell leukemia (see, e.g., Bains, T. et al. 2005 Leukemia 26, 2144-2146; Zhang, J. et al. 2012 Nature 481, 157-163; each herein incorporated by reference in its entirety), NK/T-cell leukemias (see, e.g., Koo, G. C. et al. 2012 Cancer Discov 2, 591-597; herein incorporated by reference in its entirety) and megakaryoblastic leukemias (see, e.g., Kiyoi, H., 2007 Leukemia 21, 574-576; De Vita, S. et al. 2007 Br J Haematol 137, 337-341; Malinge, S. et al. 2008 Blood 112, 4220-4226; each herein incorporated by reference in its entirety) it had not previously been reported in SS or in other mature T-cell leukemias. Indeed, the JAK1 p.Y654F mutation has not previously been reported in any human cancer. Altogether, such phosphoproteomic analysis and exome sequencing suggested that constitutive activation of the JAK-STAT pathway could play a role in the pathogenesis of mature T-cell leukemia.

### Example II

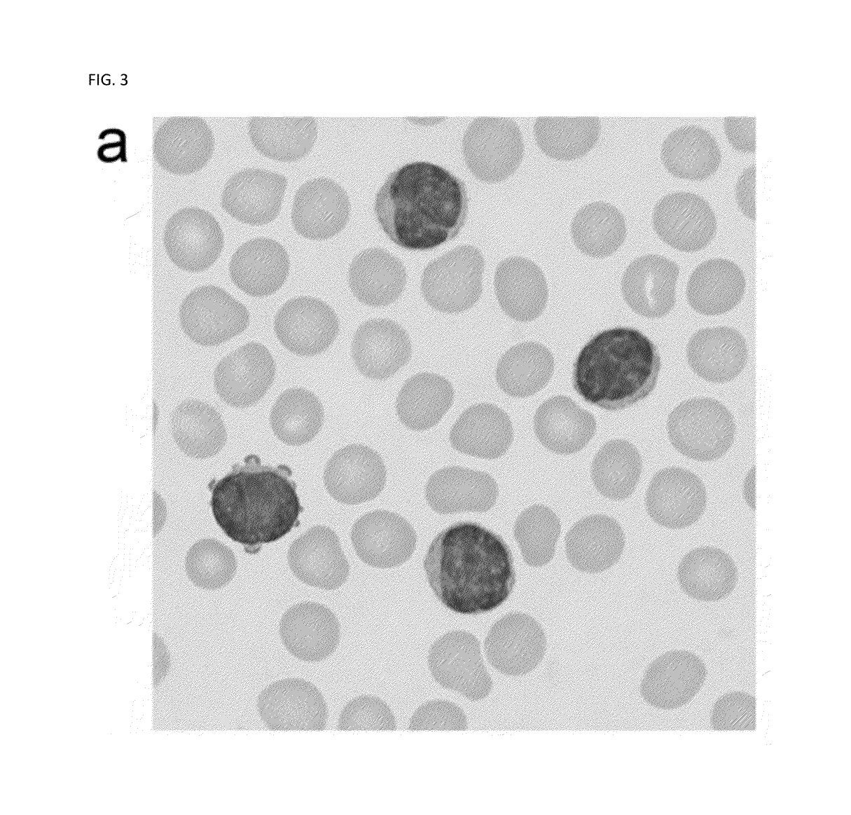

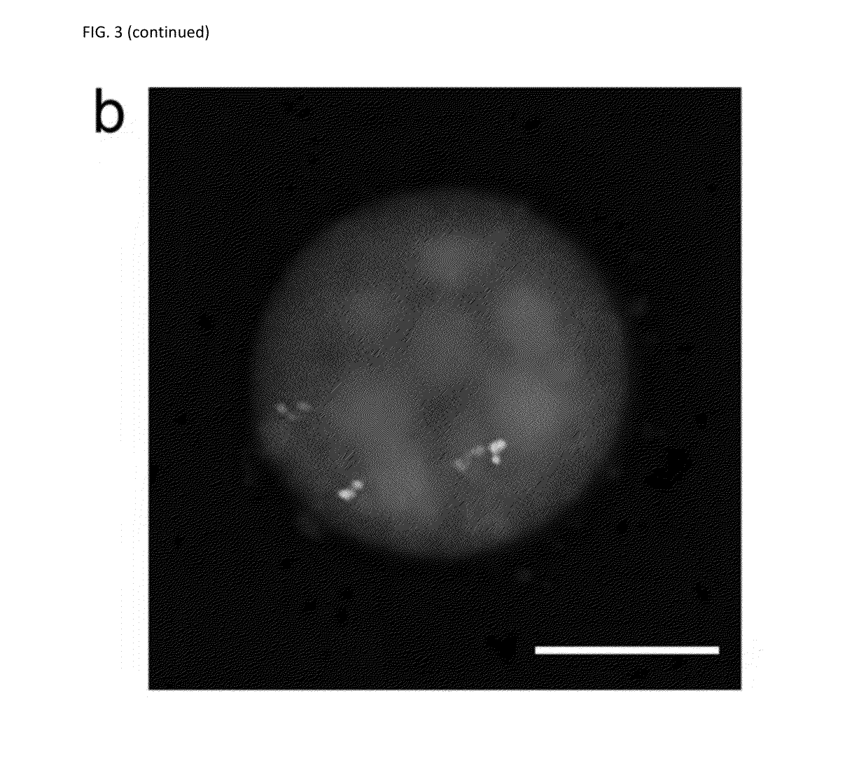

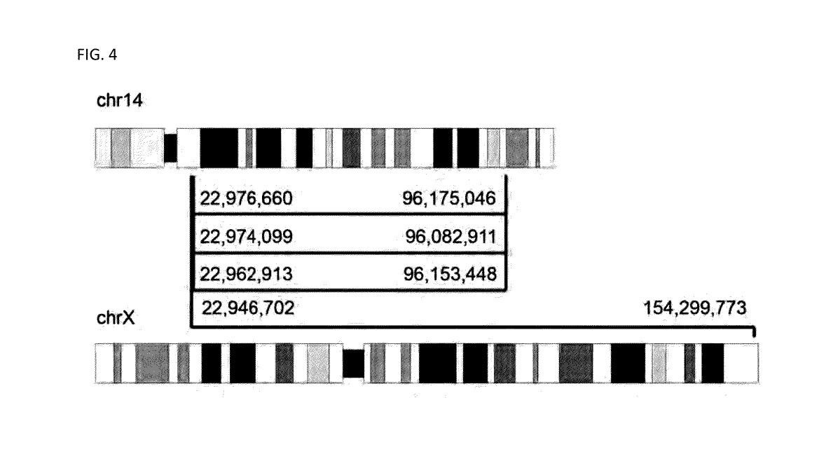

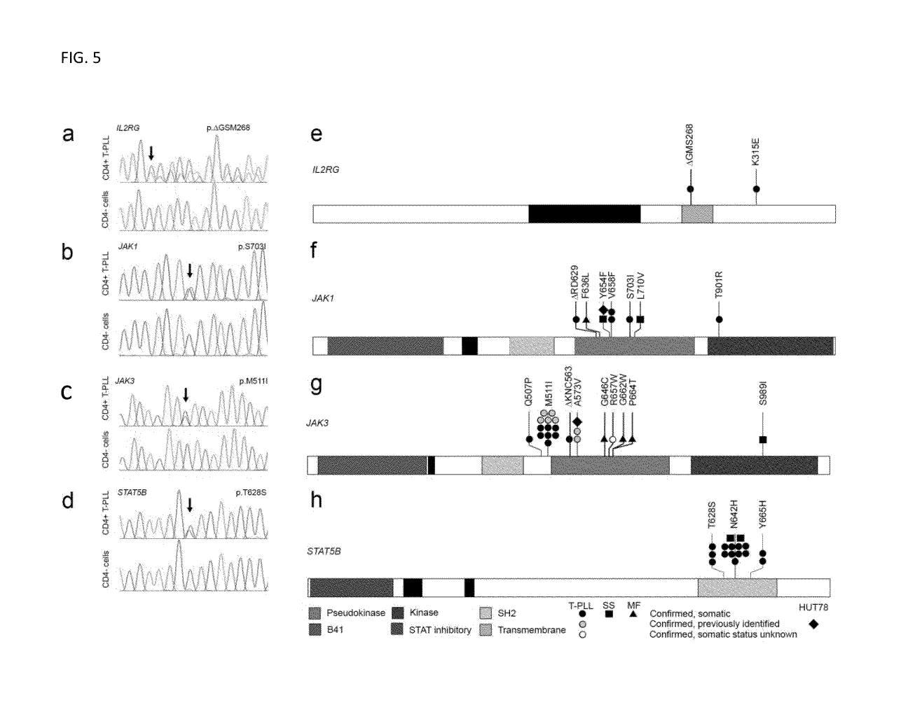

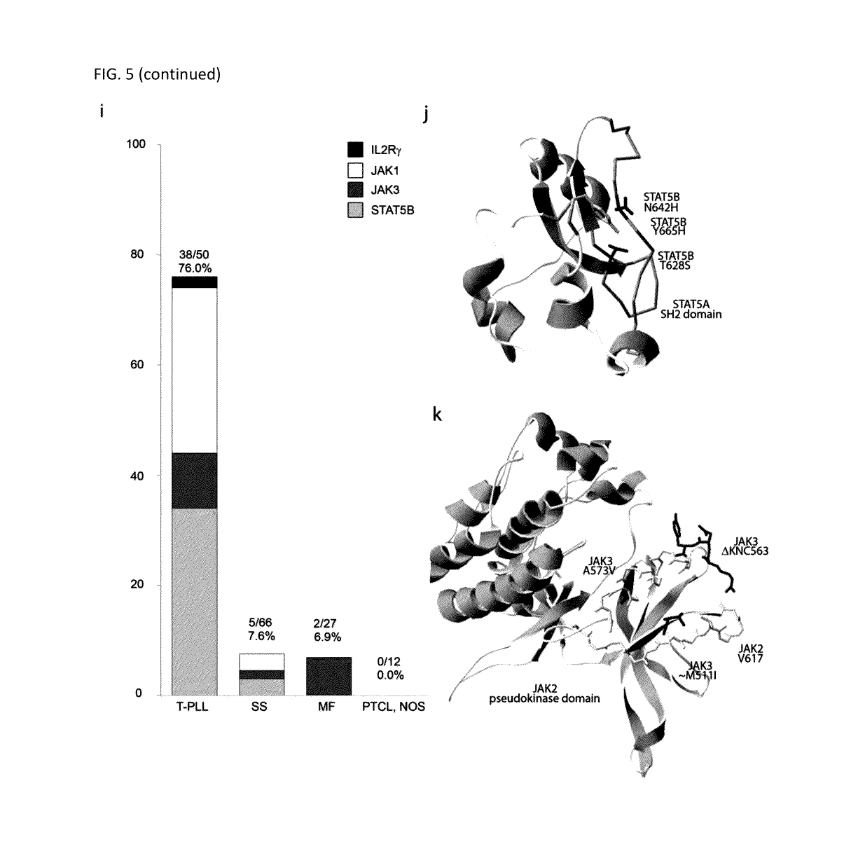

To gain further insight into the structural alterations driving JAK-STAT activation in mature T-cell leukemias, whole genome sequencing (WGS) of 4 well-characterized index cases of T-PLL selected based on fulfillment of established diagnostic criteria including possession of characteristic cytologic, immunophenotypic and karyotypic features was performed (FIG. 3). WGS confirmed presence of structural alterations involving the TCL1B locus or its analogue MTCP in all 4 cases (FIG. 4). This analysis also revealed mutations in 2 of 4 samples in JAK1 not previously been associated with T-PLL (p.V658F (see, e.g., Zhang, J. et al. 2008 Blood 118, 3080-3087; Jeong, E. G. et al. 2008 Clin Cancer Res 14, 3716-3721; each herein incorporated by reference in its entirety) and p.S703I (see, e.g., Zhang, J. et al. 2012 Nature 481, 157-163; herein incorporated by reference in its entirety), both of which were confirmed to be somatic (for p.S703I see FIG. 5b). One JAK1 mutation (p.V658F) is homologous to the JAK2 p.V617F mutation characteristic of myeloproliferative neoplasms (MPNs) and both have been reported in isolated cases of lymphoid malignancy in the COSMIC database of somatic cancer mutations. The integrated phosphoproteomic and mutational screen highlighted alterations in the JAK-STAT pathway in these samples, raising the possibility that additional genetic defects other than those previously known to characterize T-PLL might play an important role in disease pathogenesis.

### Example III

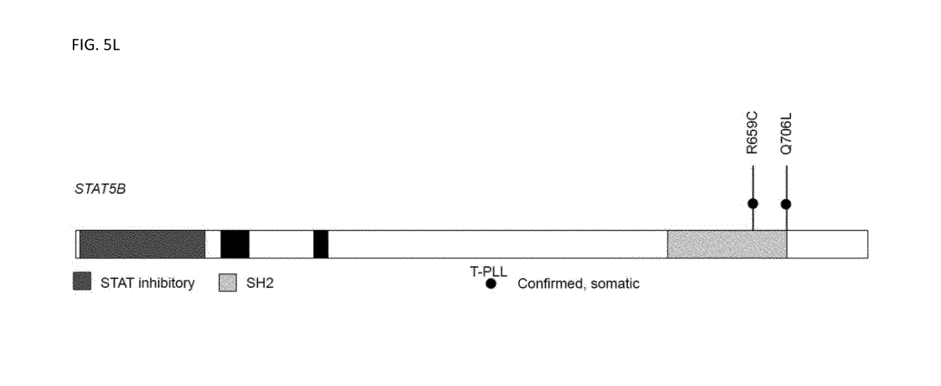

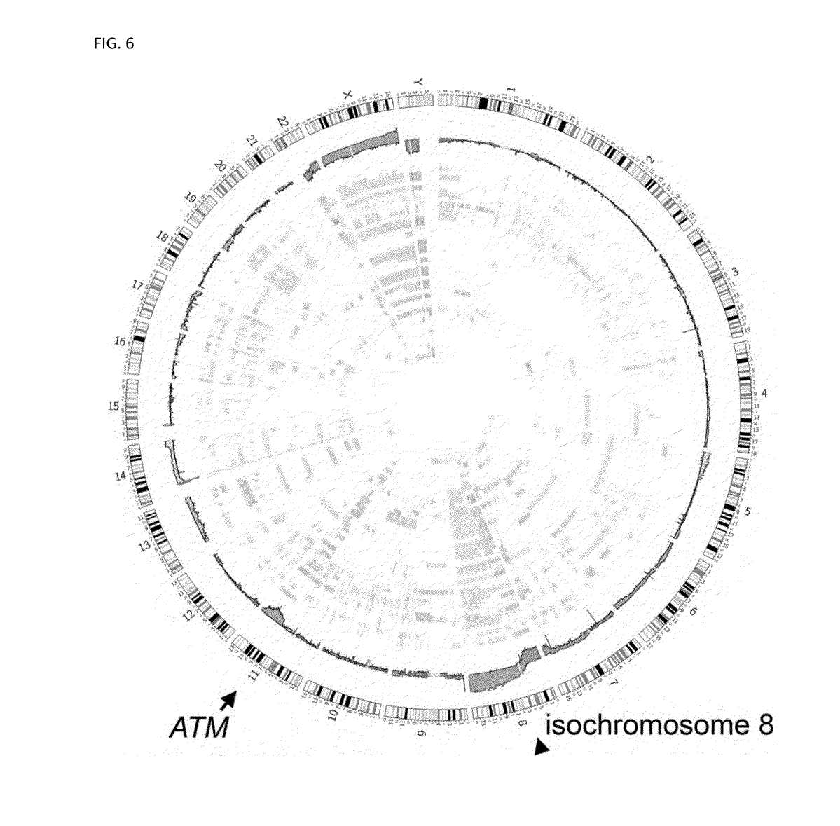

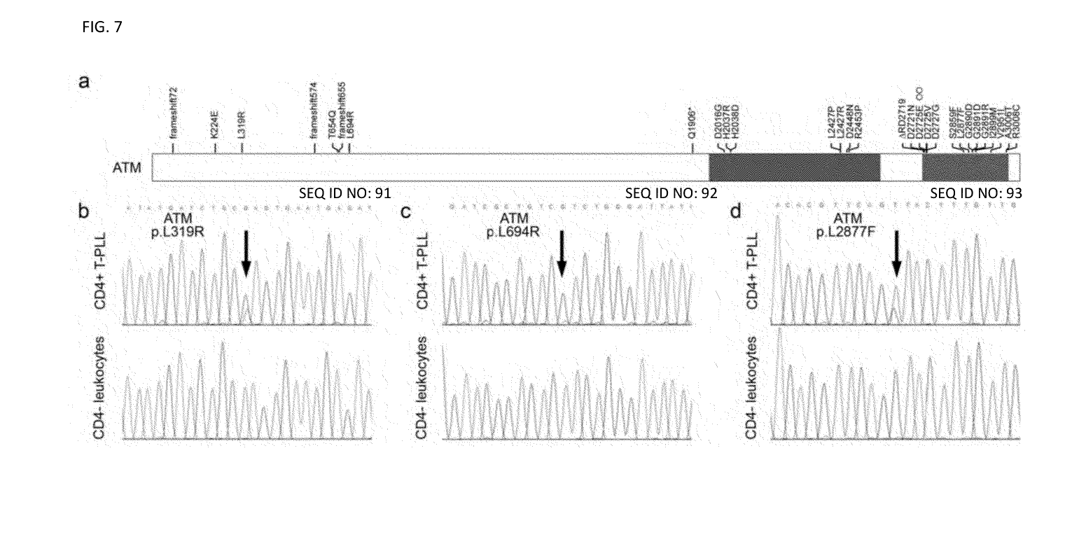

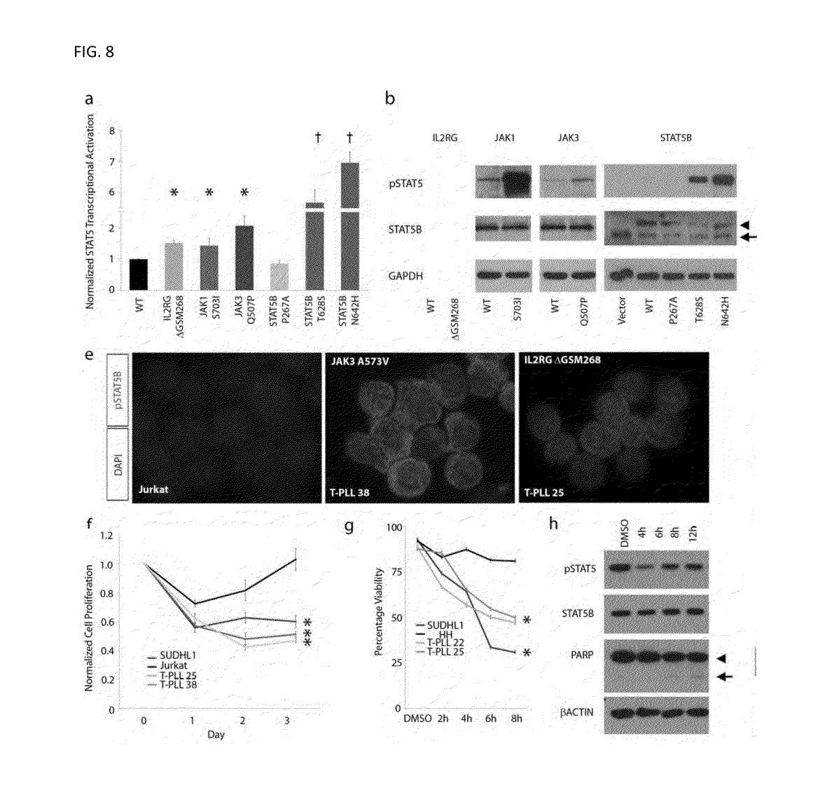

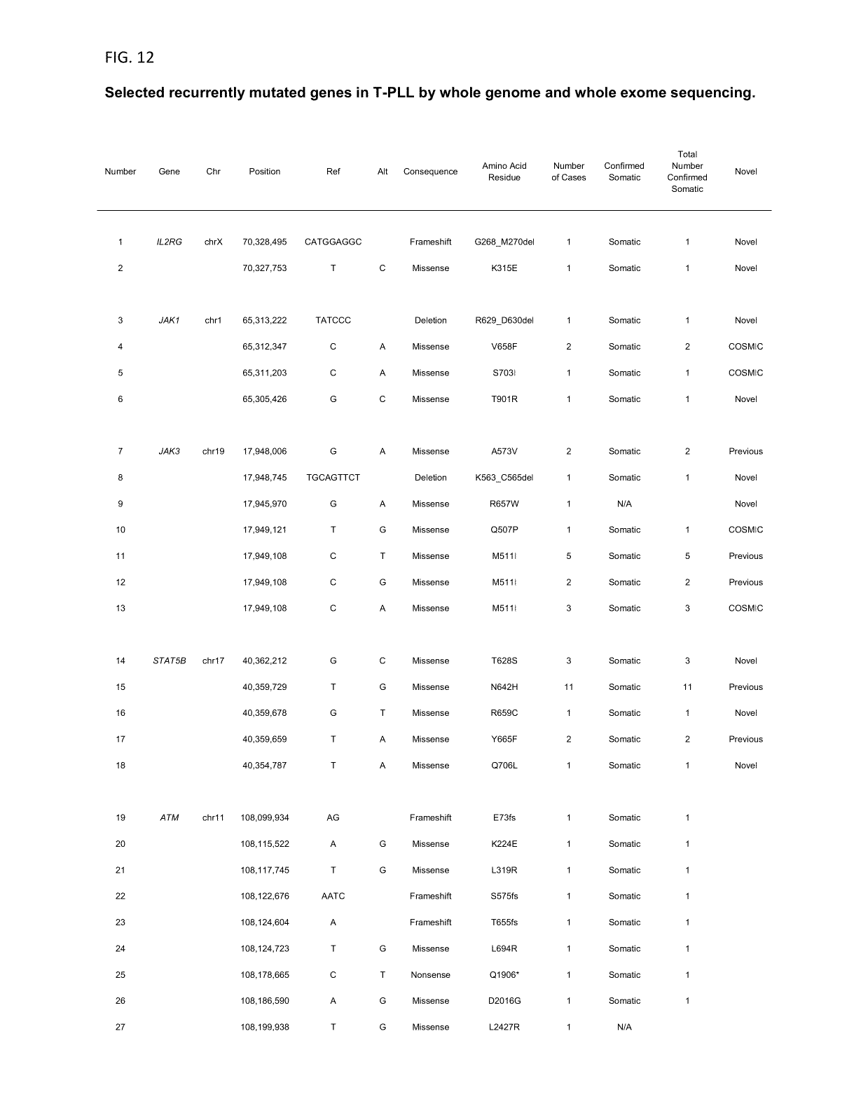

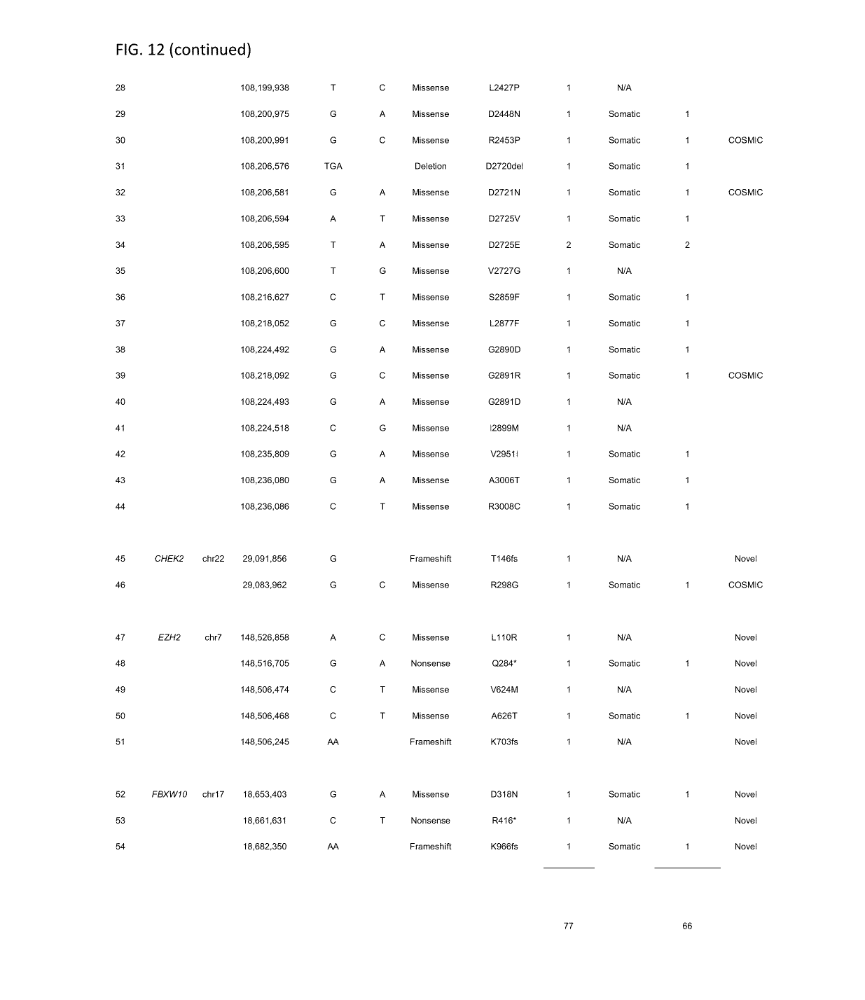

To better understand the molecular genetic basis of T-PLL, whole exome sequencing (WES) and high-resolution copy number variant (CNV; array comparative genomic hybridization, aCGH) analysis was next performed on an additional 36 and 39 T-PLL cases, respectively. Consistent with previous reports (see, e.g., Yuille, M. A. et al. 1998 Oncogene 16, 789-796, doi:10.1038/sj.one.1201603 (1998); Pekarsky, Y., et al. 1999 Proc Natl Acad Sci USA 96, 2949-2951 (1999); each herein incorporated by reference in its entirety), aCGH confirmed loss of the ATM locus at chromosome 11q in 65.1% (28/39) of all cases (FIG. 6, arrow) and isochromosome 8 or large-scale gains of chromosome 8q in 76.9% (30/39) of the T-PLL samples analyzed (FIG. 6, arrowhead). Additionally, 65.1% (28/43) of samples analyzed harbored somatic mutations in the tumor suppressor ATM (FIG. 7) predicted to be deleterious to protein function including frameshift and nonsense mutations as well as missense mutations clustered in the FAT and PI3K domains similar to previous reports (see, e.g., Stilgenbauer, S. et al. 1997 Nat Med 3, 1155-1159; Vorechovsky, I. et al. 1997 Nat Genet. 17, 96-99; each herein incorporated by reference in its entirety). Using this approach, a number of genetic alterations not previously associated with T-PLL were identified including mutations in CHEK2, a gene encoding a protein kinase activated in response to DNA damage (see, e.g., Antoni, L., et al. 2007 Nat Rev Cancer 7, 925-936; herein incorporated by reference in its entirety) (2/43, 4.7%) and novel mutations in EZH2, a member of the PcG-family of transcriptional repressors (5/43, 11.6%) frequently mutated in myeloid (see, e.g., Khan, S, N. et al. 2013 Leukemia 27, 1301-1309; herein incorporated by reference in its entirety) and lymphoid (see, e.g., Morin, R. D. et al. 2010 Nat Genet. 42, 181-185; herein incorporated by reference in its entirety) malignancy and FBWX10, a member of the F-box protein family of ubiquitin ligases (3/43, 7.0%; FIG. 12). Mutations in these proteins included several deleterious frameshift and nonsense mutations raising the possibility that ATM, CHEK2, EZH2 and FBWX10 might contribute to T-PLL pathogenesis via their roles in DNA repair, epigenetic transcriptional regulation and proteosomal degradation pathways, respectively.

Strikingly, WES analysis also identified numerous mutations in JAK1, JAK3 and STAT5B including recurrent lesions not previously associated with T-PLL (FIG. 12 and FIG. 5e-h and 1, circles). The Janus kinase/signal transducers and activators of transcription (JAK/STAT) family mediates cytokine signaling in lymphocytes and has been implicated in the pathogenesis of a number of other hematopoietic malignancies (see, e.g., Chen, E., et al. 2012 Immunity 36, 529-541; herein incorporated by reference in its entirety). Altogether, WGS, WES and targeted Sanger sequencing analysis identified mutations in JAK1 (10.0%, 5/50) and JAK3 (30.0%, 15/50; FIG. 5f-g and 5i) that were clustered in the auto-inhibitory-pseudokinase and included variants previously detected in leukemias other than mature T-cell neoplasia or shown to lead to constitutive activation of JAK-STAT signaling (see, e.g., Chen, E., et al. 2012 Immunity 36, 529-541; Knoops, L., et al. 2008 Oncogene 27, 1511-1519; each herein incorporated by reference in its entirety) (including JAK1 p.V658F (see, e.g., Zhang, J. et al. 2011 Blood 118, 3080-3087; Jeong, E. G. et al. 2008 Clin Cancer Res 14, 3716-3721; each herein incorporated by reference in its entirety) and p.S703I (see, e.g., Zhang, J. et al. 2012 Nature 481, 157-163; herein incorporated by reference in its entirety); JAK3 p.M511I (see, e.g., Walters, D. K. et al. 2006 Cancer Cell 10, 65-75; Zhang, J. et al. 2012 Nature 481, 157-163; Yamashita, Y. et al. 2010 Oncogene 29, 3723-3731; each herein incorporated by reference in its entirety), p.A573V (see, e.g., Koo, G. C. et al. 2012 Cancer Discov 2, 591-597; herein incorporated by reference in its entirety) and p.R657 (see, e.g., Yamashita, Y. et al. 2010 Oncogene 29, 3723-3731; herein incorporated by reference in its entirety). Mutations in STAT5B (34.0%, 17/50) were clustered in the SH2 domain (p.T628S, p.N642H, p.R659c, and p.Y665H; FIGS. 5h and 5l) that mediates the interaction between JAK and STAT proteins. Prior to the experiments of the present invention, high frequency STAT5B mutations have not been reported in any human cancer (see, e.g., Rajala, H. L. et al. 2013 Blood 121, 4541-4550; herein incorporated by reference in its entirety). The high homology between STAT5A and STAT5B (FIG. 7a) permitted localization of the STAT5A residues homologous to the mutated STAT5B residues (FIG. 5j) and revealed a very close 3-D proximity for these three residues in the SH2 domain to the predicted phosphotyrosine-binding loop. A novel mutation in the IL2RG transmembrane domain (p.ΔGSM268 and p.K315E; 2.0%, 1/50; FIG. 5a and e) was also identified in a single T-PLL case similar to the mutations described in IL7R in T-acute lymphoblastic leukemia (see, e.g., Zhang, J. et al. 2012 Nature 481, 157-163; herein incorporated by reference in its entirety). The experiments conducted during the course of developing embodiments for the present invention, as such, represents the first report of a somatic mutation in IL2RG in human malignancy. Additionally, a novel JAK1 mutation (p.T901R; 2.0%, 1/50; FIG. 5f) in the kinase domain was also identified. All of the mutations identified in these genes were confirmed by Sanger sequencing and where normal matched DNA was available were confirmed to be somatic (FIG. 12; FIG. 5e-h and 1, closed circles).

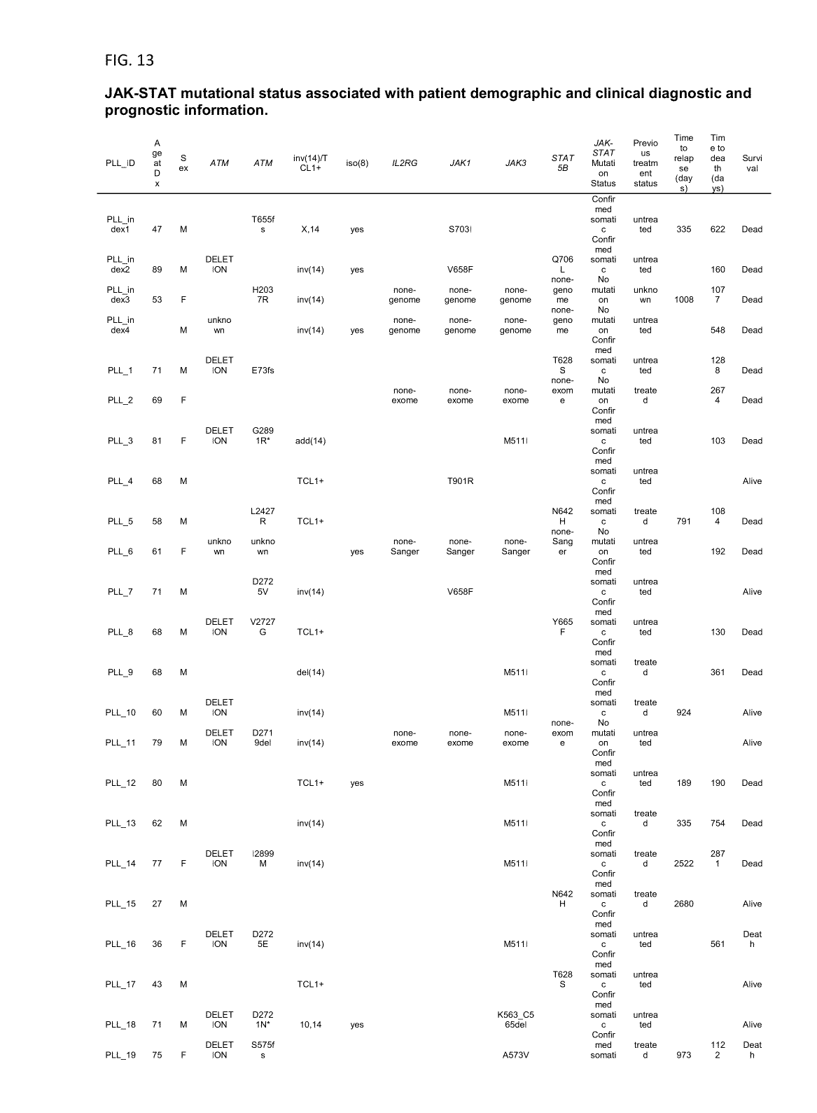

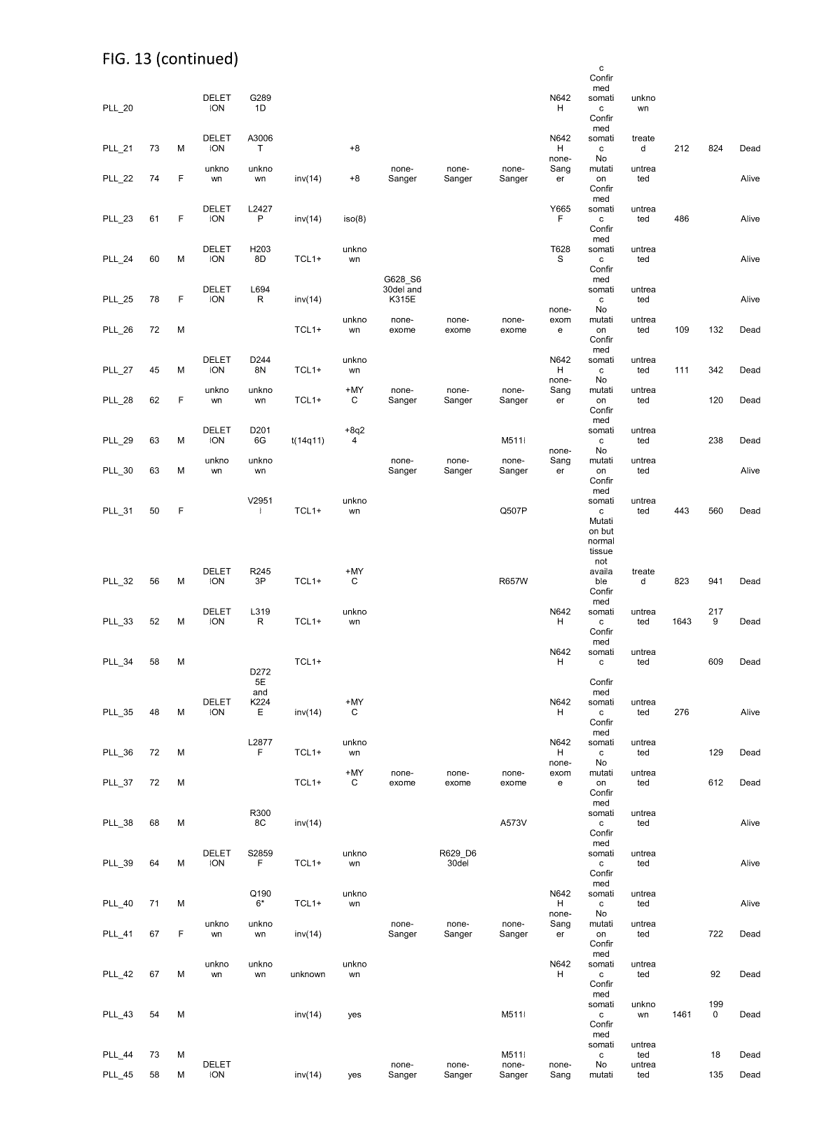

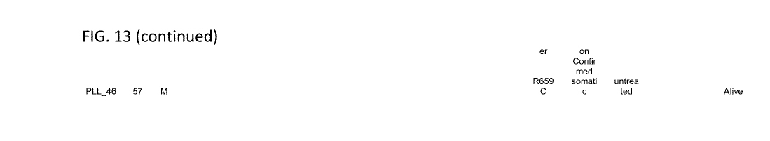

An additional p.Q706L mutation in STAT5B was also identified in a sample with a JAK1 p.V658F mutation) that mediates the interaction between JAK and STAT proteins. Significantly, mutations in STAT5B have never been previously reported in T-PLL. In the cohort, STAT5B mutations actually represented the highest frequency of all JAK-STAT family members (36.0%, FIG. 12 and FIG. 13). Notably, the WGS and WES studies of 50 cases of T-PLL revealed no evidence of activating STAT3 mutations. In total, 38 out of 50 T-PLL genomes harbored somatic mutations in IL2RG, JAK1, JAK3 or STAT5B (76.0%, FIG. 12). With one exception (JAK1 p.V658F and STAT5B p.Q706L; see FIG. 13), all cases harbored mutually exclusive mutations in genes comprising the IL2RG-JAK1-JAK3-STAT5B pathway. In the majority of patients with mutations in the JAK-STAT pathway (32/38, 84.2%), matched constitutional normal tissue (e.g. CD4-leukocytes from peripheral blood) was available to confirm the somatic status of the mutations (FIG. 12 and FIG. 13). Of the 18 distinct JAK-STAT pathway mutations identified, all but one (JAK3 p.R657W) were confirmed to be somatic (FIG. 12; 17/18, 94.4%). These data indicate a significant role for mutational activation of the IL2R-JAK1-JAK3-STAT5 axis in T-PLL.

### Example IV

To investigate whether alterations in the IL2R/JAK1/JAK3/STAT5B pathway occurred in other mature T-cell diseases, targeted Sanger sequencing of regions of recurrent mutation in IL2RG, JAK1, JAK3 and STAT5B was performed in 66 well-characterized cases of SS and 27 cases of Mycosis Fungoides (MF), the most common mature T-cell neoplasm of the skin with periodic extra-cutaneous and leukemic dissemination. This analysis detected 2 mutations in the pseudokinase domain of JAK1 (the previously identified p.Y654F mutation and the novel p.L710V mutation, FIG. 5d) and 1 additional mutation in the kinase domain of JAK3 (p.S989I, FIG. 5g) among SS samples (4.5%, 3/66; FIG. 5i) and 4 novel mutations in the JAK1 or JAK3 pseudokinase domains (JAK1 p.F636L, JAK3 p.G646C, JAK3 p.G662W, and JAK3 p.P664T; note that the p.G646C, p.G662W and p.P664T mutations are JAK3 mutations and were inadvertently identified in FIG. 5 as JAK1 mutations in the first version of FIG. 5f; FIG. 5f) in 2/27 MF samples (6.9%, FIG. 5i). An additional 2 mutations in STAT5B were also identified in 2 SS samples (p.N642H, 3.0%, 2/66; note that these mutations were inadvertently not illustrated in the first version of FIGS. 5h and 1; FIG. 5h-1,1). Mutations in IL2RG, JAK1, JAK3 or STAT5B were not identified in 16 cases of reactive lymphoid hyperplasia (not shown) nor in 12 cases of Peripheral T-cell Leukemia (PTCL, FIG. 5i). Altogether, these data indicate a high prevalence of altered JAK-STAT5 signaling in mature T-cell leukemias.

### Example V

The clustering of the JAK and STAT5B mutations strongly suggested a gain-of-function mechanism of pathogenesis. This raised the possibility that mutations identified in JAK1 and JAK3 were similar to the JAK2 p.V617F mutation. To test this, the sequence of JAK2 and JAK3, the most recurrently altered protein in the dataset, was analyzed. The crystal structure of JAK3 has not been reported however, the high degree of homology between JAK2 and JAK3 (FIG. 7c-d) prompted localization in the JAK2 3-dimensional structure the JAK2 residues analogous to the recurrent JAK3 mutations, p.M511I and p.A573V and the adjacent p.ΔKNC563 mutation. Plotting these residues onto the 3-dimensional structure of JAK2 revealed the close proximity of the p.V617 residue and residues analogous to the recurrent JAK3 mutations (FIG. 5k). These results strongly suggest that the JAK3 p.M511I mutation in T-PLL is acting similar to the JAK2 p.V617F mutation in MPN. Indeed, the p.M511I mutation has been detected in acute megakaryoblastic leukemia supporting its pathogenic role in T-PLL (see, e.g., Walters, D. K. et al. 2006 Cancer Cell 10, 65-75; herein incorporated by reference in its entirety).

### Example VI

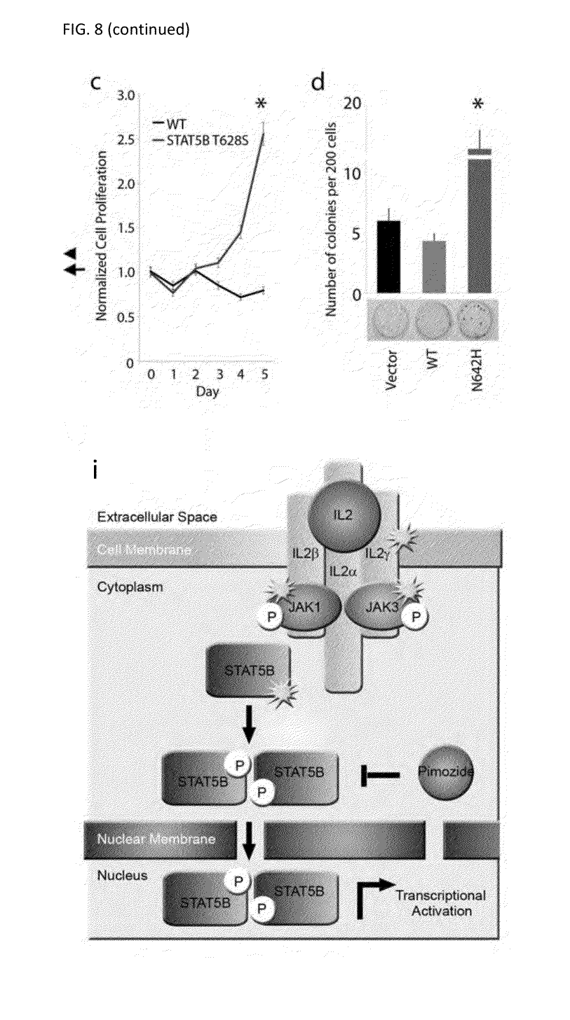

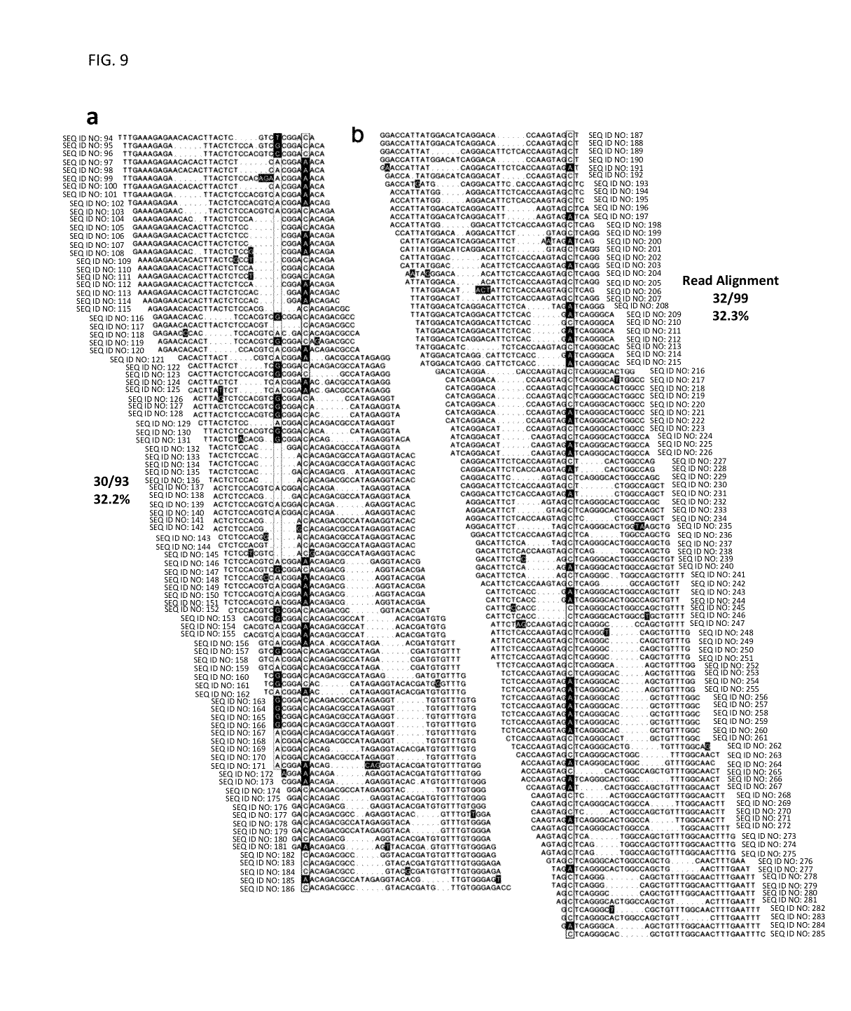

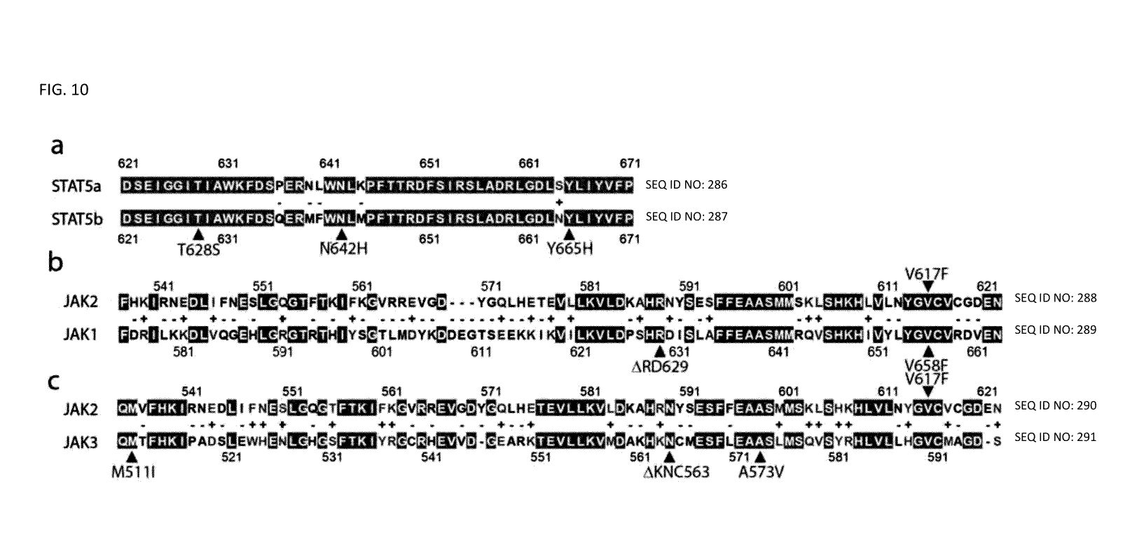

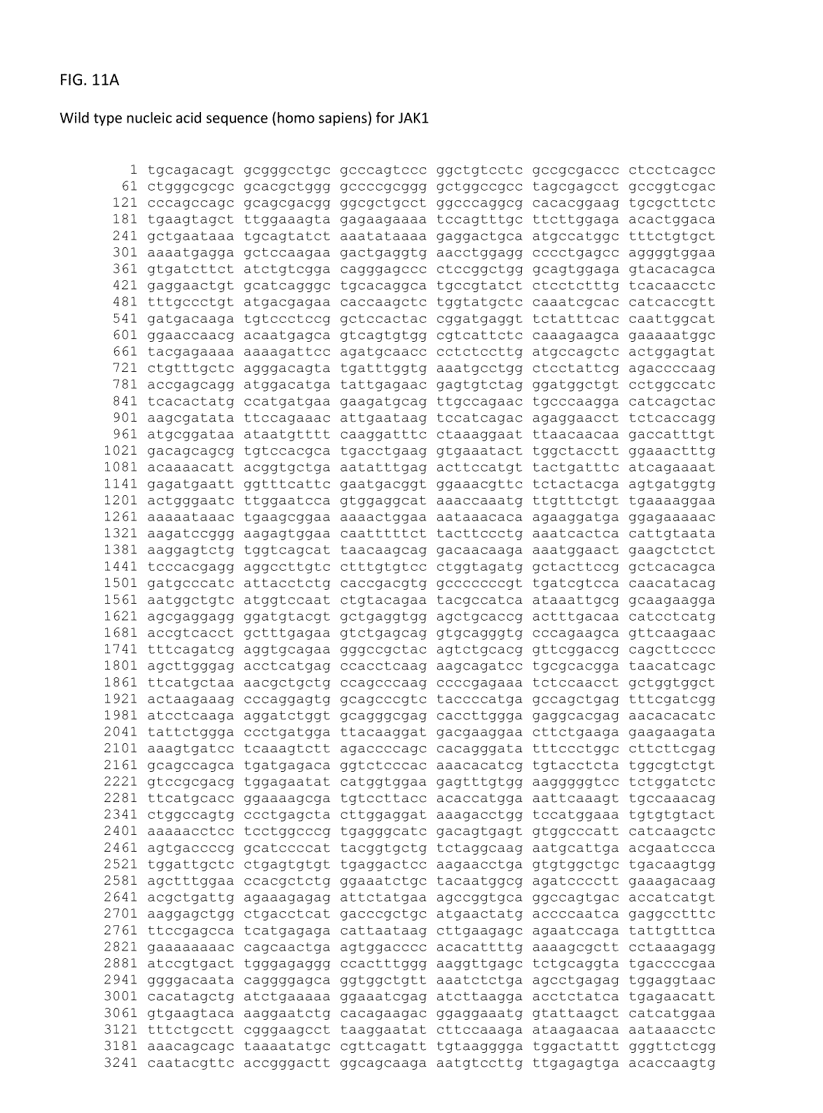

To investigate the functional effect of representative novel mutations on JAK-STAT5 activation, HeLa cells were engineered to express mutant IL2RG (p.ΔGMS628), JAK1 (p.S703I), JAK3 (p.Q705P) and STAT5B (p.T628S) proteins (FIG. 8a-b). In each case, expression of mutant IL2RG, JAK1, JAK3 and STAT5B protein led to elevated levels of STAT5 activation as determined by transcriptional reporter assays (1.4-5.7 fold increase) and pSTAT5 expression as determined by Western blot analysis (FIG. 8a-b). Expression of the previously described p.N642H mutant STAT5B protein also led to an increase in STAT5B activation as expected (FIG. 8a-b). Moreover, expression of the novel STAT5B p.T628S mutant protein lead to increased cell proliferation in Ba/F3 cells (FIG. 8c). In addition, STAT5B p.N642H significantly increased colony-forming capacity in the Jurkat T-cell line (FIG. 8d). Immunofluorescence microscopy on primary T-PLL cases harboring IL2RG, JAK3 or STAT5B mutations showed elevated total cellular levels and nuclear localization of pSTAT5B protein (data shown for representative IL2RG and JAK3 mutated primary cells; FIG. 8e) illustrating a common mechanism of pathogenesis in both IL2RG-, JAK- and STAT5B-mutated T-PLL cases.

### Example VII

Given the convergence of common gamma chain (IL2RG), JAK1 and JAK3 signaling on STAT5B activation and the established role of STAT5 in cytokine-induced peripheral T-cell proliferation (see, e.g., Constantinescu, S. N., et al. 2008 Trends Biochem Sci 33, 122-131; herein incorporated by reference in its entirety), the effect of specific STAT5 inhibition on primary T-PLL and SS cells harboring IL2RG, JAK1/3 or STAT5B mutations was determined. Treatment of cultured primary T-PLL cells with a selective STAT5 inhibitor (Pimozide) previously shown to reduce STAT5 levels in myeloid leukemia (see, e.g., Nelson, E. A. et al. 2011 Blood 117, 3421-3429; herein incorporated by reference in its entirety) resulted in significant and specific reduction in leukemia cell proliferation (FIG. 80 and viability (FIG. 8g-h; arrow in FIG. 8h indicates PARP-mediated apoptotic activation) and lead to a dose- and time-dependent reduction in pSTAT5B levels (FIG. 8h).

### Example VIII

This example provide the materials and methods for Examples I-VII.

DNA from primary T-PLL samples was subjected to WGS, WES and 270K feature aCGH. HUT78 cells were subjected to WES and phosphoproteomic analyses. SS, MF, PTCL and reactive lymphoid hyperplasia samples were subjected to targeted Sanger sequencing of selected regions of IL2RG, JAK1, JAK3 and STAT5B. Confirmation of selected mutations identified by WGS and WES was performed by Sanger sequencing of tumor DNA as well as any available DNA from constitutional normal samples. Primary T-PLL and HUT78 cells were cultured in the presence of IL2 with the addition of the specific STAT5 inhibitor Pimozide. Mutational analysis was performed in HeLa and Jurkat cell lines.

Patients Samples and DNA Extraction

All T-PLL cases fulfilled pathologic criteria for diagnosis of T-PLL according to World Health Organization classification criteria without knowledge of JAK/STAT mutational status. For a given patient, samples represented either formalin-fixed paraffin embedded tissue (FFPE), cryopreserved peripheral blood leukocytes or both. Where applicable, constitutional normal tissue represented either tumor-free FFPE tissue derived from the same patient or otherwise tumor depleted peripheral blood leukocytes generated using EasySep column enrichment and B220 and/or Mac-1 positive cell selection (Stem Cell Technologies, Inc.). Relative tumor-depletion of resultant cell suspensions was determined by flow cytometry using antibodies directed against CD4 (BD Pharmingen). DNA was extracted from both FFPE and frozen samples using QIAGEN DNA extraction kits according to manufacturer's instructions.

Protein Extraction and Digestion

Approximately sixty million cells were lysed in buffer containing 9 M urea/20 mM HEPES pH8.0/0.1% SDS and a cocktail of phosphatase inhibitors. For each sample, 6 mg of protein were reduced with 4.5 mM DTT and then alkylated with 10 mM iodoacetamide. Samples were diluted 5-fold with 20 mM HEPES and then digested with trypsin overnight at 37° C. using an enzyme-to-protein ratio of 1/50 (w/w). Samples were desalted on a C18 cartridge (Sep-Pak plus C18 cartridge, Waters), then purified peptides were dried before further processing.

Phosphopeptide Enrichment

Metal oxide affinity chromatography (MOAC) was performed to enrich phosphorylated peptides and reduce the sample complexity prior to tyrosine-phosphorylated peptide immunopurification (pY-IP). Titanium dioxide (TiO2) microparticles (Titansphere® Phos-TiO, GL Sciences Inc.) applying a TiO2 microparticles-to-protein ratio of 6/1 (w/w) was used. Briefly TiO2 microparticles were conditioned with the buffer A (80% ACN/0.4% TFA), then equilibrated with the buffer B (75% buffer A/25% lactic acid). Peptides were solubilized with 200 μl buffer A and mixed with 400 μl buffer B then loaded twice on TiO2 microparticles. Microparticles were washed 2 times with buffer B and 3 times with buffer A. Hydrophilic phosphopeptides were eluted with 5% ammonium hydroxide solution and hydrophobic phosphopeptides were eluted with 5% pyrrolidine solution. After elution, peptides were dried using a SpeedVac. The equivalent of 5 mg of protein was further enriched for phosphorylated tyrosine peptides by overnight immunoprecipitation (pY-IP) using a cocktail of anti-phosphotyrosine antibodies (4G10, Millipore/PT-66, Sigma/p-Tyr-100, Cell Signaling Technology). After elution, phosphotyrosine peptides were dried.

Mass Spectrometry

Ammonium hydroxide and pyrrolidine eluents were dried and reconstituted in 25 μl loading buffer (0.1% TFA/2% acetonitrile). Eluents from pY-IP were dried and reconstituted in 35 μl loading buffer. An LTQ Orbitrap XL (ThermoFisher) in-line with a Paradigm MS2 HPLC (Michrom bioresources) was employed for acquiring high-resolution MS and MS/MS data. Ten microliters of the phospho-enriched peptides were loaded onto a sample trap (Captrap, Bruker-Michrom) in-line with a nano-capillary column (Picofrit, 75 μm i.d.x 15 μm tip, New Objective) packed in-house with 10 cm of MAGIC AQ C18 reverse phase material (Michrom Bioresource). Two different gradient programs, one each for MOAC and pY-IP samples, were used for peptide elution. For MOAC samples, a gradient of 5-40% buffer B (95% acetonitrile/1% acetic acid) in 135 min and 5 min wash with 100% buffer B followed by 30 min of re-equilibration with buffer A (2% acetonitrile/1% acetic acid) was used. For pY-IP samples a 5-40% gradient with buffer B was achieved in 75 min followed by 5 min wash with buffer B and 30 min re-equilibration. Flow rate was ˜0.3 μl/min. Peptides were directly introduced into the mass spectrometer using a nano-spray source. Orbitrap was set to collect 1 MS scan between 400-2000 m/z (resolution of 30,000 @ 400 m/z) in orbitrap followed by data dependent CID spectra on top 9 ions in LTQ (normalized collision energy ˜35%). Dynamic exclusion was set to 2 MS/MS acquisitions followed by exclusion of the same precursor ion for 2 min. Maximum ion injection times were set to 300 ms for MS and 100 ms for MS/MS. Automatic Gain Control (AGC) was set to 1 xe6 for MS and 5000 for MS/MS. Charge state screening was enabled to discard +1 and unassigned charge states. Technical duplicate data for each of the MOAC eluents (ammonium hydroxide and pyrrolidine) and triplicate data for the pY-IP eluents were acquired.

Bioinformatics Analysis

RAW files were converted to mzXML using msconvert and searched against the Swissprot Human taxonomic protein database (2013Jan., 09 release) appended with common proteomics contaminants and reverse sequences as decoys. Searches were performed with X!Tandem (version 2010.10.01.1) using the k-score plugin. For all searches the following parameters were used: A parent monoisotopic mass error window of 50 ppm was used. The fragment ion error window was 0.8 Da. Searches were performed allowing for up to 2 missed tryptic cleavages. Potential modifications of oxidation of Methionine (+15.9949@M), carbamidomethylation of Cysteine (+57.0214@C), and phosphorylation of Serine, Threonine, and Tyrosine (+79.9663@[STY]) residues were allowed. The search results were then post-processed using PeptideProphet and ProteinProphet.

Mass spectral counts delivered through the Trans-Proteomic Pipeline (TPP) phosphoproteomic analyses for each cell line using the spectral counting software ABACUS.

Tyrosine-enrichment data were processed through ABACUS separately from the Serine and Threonine data. ABACUS results were filtered to only retain those proteins with a ProteinProphet probability greater than 0.7. Only peptides with a probability greater than 0.7 and containing a phosphorylation on a serine, threonine or tyrosine were considered for spectral counting. For the tyrosine enrichment data no decoy proteins were reported when using these ABACUS parameters. Label-free spectral counting was used from the ABACUS output in all further analysis to quantify the relative abundance of phosphorylated peptides/proteins. The proteins reported by ABACUS were then manually curated to select the best representative protein identifier from among ambiguous cases. Phospho-site localization was performed with an in-house reimplementation of the Ascore algorithm as described (see, e.g., Beausoleil, S. A., et al. 2006 Nat Biotechnol 24, 1285-1292; herein incorporated by reference in its entirety). Recurrent phosphorylation site motifs were extracted with the Motif-X algorithm. Sequences were uploaded with the phosphorylated residue at the central position and six amino acids on each side. The minimum motif occurrences was set to 20, with a p<10-5 required to be rated as significant.

High-Throughput and Sanger DNA Sequencing

For WGS, 7-10 μg of high-molecular-weight genomic DNA was extracted from fresh frozen tumor tissue and subjected to WGS by Complete Genomics, Inc. (CGI; Mountain View, Calif.). CGI performs massively parallel short-read sequencing using a combinatorial probe-anchor ligation (cPAL) chemistry coupled with a patterned nanoarray-based platform of self-assembling DNA nanoballs (Drmanac et al., 2010). Library generation, read-mapping to the NCBI reference genome (Build 37, RefSeq Accession nos. CM000663-CM00686), local de novo assembly and variant-calling protocols were performed as previously described (Drmanac et al., 2010; Roach et al., 2010). Initial read mapping and variant calling were performed using CGAtools v1.3.0 (http://cgatools.sourceforge.net/docs/1.3.0/). Additional downstream bioinformatic analyses of WGS data were performed using custom designed processing routines. WGS yielded a mean of 351±13 Gb mapped per sample with 97.4-97.8% fully called genome fraction and 97.1-97.7% fully called exome fraction. The median genomic sequencing depth exceeded 60× in all samples normalized across the entire genome. Three of the 4 index T-PLL cases harbored the characteristic inv(14) alteration as anticipated based on clinical karyotyping and confirmatory FISH results and the fourth harbored the less common t(X;14) lesion (FIGS. 2 and 3).

For WES, genomic DNA samples were fragmented using a Covaris S2 fragmentation system to a target size of 400 bp. The samples were end-repaired, a-tailed, and custom adapters were ligated using the NEBNext® DNA Library Prep kit according to the manufacturers recommended protocols. The custom adapters included 6 bp barcodes and synthesized by Integrated DNA Technologies (IDT). After ligation, the samples were size selected to 400 bp on a 2% agarose gel and 1 mm gel slices were retained. Samples were isolated from the gel using the Qiagen QIAquick gel extraction system. Seven microliters of each ligation product was enriched using the Phusion master mix kit and custom PCR primers with a total of 14 cycles of PCR amplification. Two PCR reactions were performed for each sample. The PCR products were pooled and purified using AmpureXP® beads.

Library QC was performed using the Agilent Tapestation and qPCR. Based on qPCR concentrations, 200 ng of each of five samples were pooled for a total of three pools. Each pool was captured using the Nimblegen SeqCap EZ V3 Exome Enrichment Kit according to the manufacturer's recommended protocols. The capture pools were combined and sequenced on the Illumina HiSeq 2000 platform across four lanes with paired-end 100 bp reads. The average depth of coverage for exome sequencing was 30.4±11.9× with greater than 95% of coding exons sequenced to a depth of 30 or more.

The analysis focused on variations that were not in the Database of SNPs (dbSNP) and/or those that had previously been associated with cancer based on information in the Catalogue of Online Somatic Mutations in Cancer (COSMIC) database. After identifying candidate mutations of interest, 10-50 ng of genomic DNA from tumor and, where available, matched constitutional tissue or otherwise tumor-depleted, matched normal DNA was subjected to Sanger sequencing. Where available, tumor-depleted matched normal DNA was also subjected to WES to identify somatic alterations in T-PLL. For all such sequencing reactions, PCR amplification was performed using Phusion DNA polymerase (New England Biolabs) followed by conventional Sanger sequencing technology using BigDye version 3.1 chemistry run on an Applied Biosystems 3730x1 DNA Sequencer at the University of Michigan DNA sequencing Core. All sequencing reactions were performed using nested sequencing primers. Sequencing trace analysis was performed using Mutation Surveyor software. All primers were designed using a custom-developed program and purchased from IDT lyophilized in 96-well plates.

Bioinformatic Processing of WES Data

WES FASTQ sequencing data files were aligned to human NCBI Build 36 reference sequence using BWA 0.6.2. Merging and deduplication was performed using Picard 1.79. The DepthOfCoverage, CountReads, RealignerTargetCreator, IndelRealigner, BaseRecalibrator, PrintReads and UnifiedGenotyper functions within GenomeAnalysisTK-1,6-9 were used to ascertain coverage and for variant calling. Resultant VCF files were annotated using snpEff v3.1. Variants previously identified as somatic as presented in COSMIC were highlighted using a custom-designed algorithm.

Array CGH

Copy-number variants (CNVs) were detected using Nimblegen whole genome arrays containing 270,000 features (Roche Applied Science) sufficient to detect CNVs of 50 kb or greater. Arrays were prepared according to manufacturer's protocol and analyzed on NimbleGen MS 200 Scanner followed by data extraction, normalization and processing for segMNT analysis using NimbleScan software according to manufacturer's instructions. Downstream data processing and interpretation was performed using custom designed processing algorithms solely reliant on the data comprising output segMNT data files. Circos plots were generated to display the segMNT data.

Mutation Analysis

JAK1 and STAT5B mutants were made using site-directed mutagenesis PCR. Wild-type and mutant genes were cloned into the pCAGGS mammalian expression vector. The mutations were confirmed by Sanger sequencing of the plasmid DNA. For STAT5B reporter assays, HeLa cells were plated in 24 well plates and transiently transfected with either of pCAGGS STAT5B or JAK1 plasmids (400 ng/well) along with pGL4.52 (Luc2P/STAT5RE/Hygro) (Promega, 400 ng/well) using PolyJet (Signagen, 2.4 μl/well). After 36 hours, the cells were lysed and One-Glo luciferase detection reagent (Promega) was used to determine luciferase activity according to the manufacturer's recommendations. The lysates from the assay were used for determining pSTAT5B levels by Western blotting. STAT5B wild-type and mutants were subcloned in pLVX Ac GFP1 lentiviral mammalian expression vector (Clontech). Viral particles were generated by co-transfecting PMD2.G, psPAX2 packaging plasmids. Targeted cells were transduced with virus for 48 hrs, followed by selection with puromycin (2 g/mL) for 2 days. The selected cells were then tested for the expression of genes by Western blotting. These cells were then used for WST (Roche) and colony forming cells assay using MethoCult soft agar (StemCell Technologies) as per manufacturer's protocol.

Immunocytochemistry

Cultured suspension cells were deposited onto glass slides by cytocentrifugation. Cells were fixed and permeabilized with methanol. After fixation, cells were first incubated with Y694/Y699 phospho-STAT5 rabbit monoclonal antibody (D47E7, Cell Signaling) followed by Alexa-conjugated donkey anti-rabbit IgG (A1exa594, Life Technologies). Coverslips were mounted on standard slides with mounting media supplemented with 4′,6′-diamidino-2-phenylindole (DAPI). The images were captured and recorded using an Olympus BX-51 upright light microscope equipped with an Olympus DP-70 camera.

Primary Cell Culture and Pimozide Treatment

Cell lines and primary cells were thawed and incubated overnight at 37° C. in cell culture medium (RPMI 1640 with 20% heat-inactivated fetal bovine serum, glutamine and antibiotics) followed by treatment with Pimozide (BioVision, diluted in DMSO vehicle), a specific STAT5 inhibitor, at a final concentration of 10-20 μM. Cells were harvested at different time points (4-12h), lysed in Laemmli buffer and incubated at 95° C. for 10 min. Protein lysates (20 μg of protein) were separated by SDS-PAGE electrophoresis, transferred to a PVDF membrane and probed with specific primary antibodies including The anti-pSTAT5 (1:1000 dilution), anti-STAT5 (1:1000 dilution) and anti-PARP (1:1000 dilution) rabbit antibodies (Cell Signaling Technology). Human leukemic T-cell line HH and Mac-2a were used as negative and positive controls, respectively. A trypan blue exclusion assay was also carried out in triplicate to monitor the cell viability over time.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention that are obvious to those skilled in the relevant fields are intended to be within the scope of the present invention.

### Incorporation by Reference

The entire disclosure of each of the patent documents and scientific articles referred to herein is incorporated by reference for all purposes.

### EQUIVALENTS

The invention may be embodied in other specific forms without departing from the spirit or essential characteristics thereof. The foregoing embodiments are therefore to be considered in all respects illustrative rather than limiting the invention described herein. Scope of the invention is thus indicated by the appended claims rather than by the foregoing description, and all changes that come within the meaning and range of equivalency of the claims are intended to be embraced therein.

1. A method for detecting one or more JAK/STAT pathway variants associated with a mature T-cell leukemia in a human subject, comprising:
a) contacting a biological sample from a subject with a JAK/STAT pathway variant detection assay under conditions that the presence of a JAK/STAT pathway variant associated with a mature T-cell leukemia is determined; and
b) diagnosing the subject with a mature T-cell leukemia when one or more of the JAK/STAT pathway variants are present in the biological sample, wherein the one or more JAK/STAT pathway variants encodes a loss of function mutation and/or a gain of function mutation.

2. The method of claim 1, wherein the JAK/STAT pathway variant is one or more variants selected from JAK1, JAK3, STAT5B, and IL2RG,
wherein the JAK1 variant is one or more JAK1 mutations selected from the group consisting of JAK1 p.F636L, JAK1 p.G646C, JAK1 p. Y654F, JAK1 p.V658F, JAK1 p.S703I, and JAK1 p.T901R,
wherein the JAK3 variant is one or more JAK3 mutations selected from the group consisting of JAK3 p.ΔKNC563, JAK3 p.M511I, JAK3 p. A573V, JAK3 p.R657, JAK3 p.G662W, JAK3 p.P664T, JAK3 p.Y980, JAK3 p. Y981, and JAK3 p.S989I,
wherein the STAT5B variant is one or more STAT5B mutations selected from the group consisting of STAT5B p.T628S, STAT5B p.R659c, STAT5B p.Q706L, STAT5B p.N642H, STAT5B p.Y699, and STAT5B p.Y665H,
wherein the IL2RG variant is one or more IL2RG mutations selected from the group consisting of IL2RG p.ΔGSM268, IL2RG p.Y325, and IL2RG p. K315E.

3. The method of claim 1,
wherein if the mature T-cell leukemia is T-cell prolymphocytic leukemia, then the one or more JAK/STAT pathway variants are selected from the group consisting of JAK1 p.V658F, JAK1 p.S703I, JAK1 p.T901R, JAK3 p.ΔKNC563, JAK3 p.M511I, JAK3 p. A573V, JAK3 p.R657, STAT5B p.T628S, STAT5B p.R659c, STAT5B p.Q706L, STAT5B p.N642H, STAT5B p.Y665H, IL2RG p.ΔGSM268, and IL2RG p. K315E,
wherein if the mature T-cell leukemia is Sezary syndrome, then the one or more JAK/STAT pathway variants are selected from the group consisting of JAK1 p. Y654F, JAK3 p. A573V, JAK3 p.Y980, JAK3 p. Y981, JAK3 p.S9891, STAT5B p.Y699, STAT5B p.N642H, and I12RG p.Y325.

4. The method of claim 1, wherein the determining comprises detecting variant JAK1, JAK3, STAT5B, and IL2RG nucleic acids or polypeptides, wherein the detecting variant JAK1, JAK3, STAT5B, and IL2RG nucleic acids comprises one or more nucleic acid detection method selected from the group consisting of sequencing, amplification and hybridization.

5. The method of claim 1, wherein the biological sample is selected from the group consisting of a tissue sample, a cell sample, and a blood sample.

6. The method of claim 1, wherein the determining comprises a computer implemented method, wherein the computer implemented method comprises analyzing JAK1, JAK3, STAT5B, and IL2RG variant information and displaying the information to a user.

7. The method of claim 1,
further comprising the step of 1) treating the subject for a mature T-cell leukemia and monitoring the subject for the presence of JAK1, JAK3, STAT5B, and IL2RG variants associated with the mature T-cell leukemia, and/or 2) treating the subject for a mature T-cell leukemia under condition such that at least one symptom of the mature T-cell leukemia is diminished or eliminated,
wherein the treating comprises inhibiting JAK1, JAK3, STAT5B, and/or IL2RG expression and/or activity,
wherein the inhibiting STAT5B expression and/or activity is accomplished through administration of an agent configured to inhibit STAT5B expression pimozide,
wherein the inhibiting JAK1 and/or JAK3 expression and/or activity is accomplished through administration of an agent configured to inhibit JAK1 and/or JAK3 expression and/or activity, wherein the agent is selected from the group consisting of ruxolitinib, tofacitinib, baricitinib, CYT387, and lestaurtinib.

8. The method of claim 7, further comprising administering one or more agents for treating a mature T-cell leukemia, wherein the one or more agents is selected from the group consisting of pentostatin, fludarabine, cladrbine, chlorambucil, cyclophosphamide, doxorubicin, vincristine, prednisone (CHOP), cyclophosphamide, vincristine, prednisone (COP), and vincristine, doxorubicin, prednisone, etoposide, cyclophosphamide, bleomycin, alemtuzumab, and vorinostat.

9. The method of claim 1, further comprising the step of detecting a variant in one or more additional genes associated with a mature T-cell leukemia, wherein the one or more genes are selected from the group consisting of CHEK2, EZH2, and FBXW10.

10. Use of a variant JAK/STAT pathway nucleic acid or polypeptide for detecting a mature T-cell leukemia in a human subject,
wherein the JAK/STAT pathway variant encodes a loss of function mutation and/or a gain of function mutuation,
wherein the JAK/STAT pathway variant is one or more variants selected from JAK1, JAK3, STAT5B, and IL2RG.

11. The use of claim 10,
wherein the JAK1 variant is one or more JAK1 mutations selected from the group consisting of JAK1 p.F636L, JAK1 p.G646C, JAK1 p. Y654F, JAK1 p.V658F, JAK1 p.S703I, and JAK1 p.T901R,
wherein the JAK3 variant is one or more JAK3 mutations selected from the group consisting of JAK3 p.ΔKNC563, JAK3 p.M511I, JAK3 p. A573V, JAK3 p.R657. JAK3 p.Y980, JAK3 p.G662W, JAK3 p.P664T, JAK3 p. Y981, and JAK3 p.S989I,
wherein the STAT5B variant is one or more STAT5B mutations selected from the group consisting of STAT5B p.R659c, STAT5B p.Q706L, STAT5B p.T628S, STAT5B p.N642H, STAT5B p.Y699, and STAT5B p.Y665H,
wherein the IL2RG variant is one or more IL2RG mutations selected from the group consisting of IL2RG p.ΔGSM268, IL2RG p.Y325, and IL2RG p. K315E.

12. The use of claim 10,
wherein if the mature T-cell leukemia is T-cell prolymphocytic leukemia, then the one or more JAK/STAT pathway variants are selected from the group consisting of JAK1 p.V658F, JAK1 p.S703I, JAK1 p.T901R, JAK3 p.ΔKNC563, JAK3 p.M511I, JAK3 p. A573V, JAK3 p.R657, STAT5B p.T628S, STAT5B p.R659c, STAT5B p.Q706L, STAT5B p.N642H, STAT5B p.Y665H, IL2RG p.ΔGSM268, and IL2RG p. K315E,
wherein if the mature T-cell leukemia is Sezary syndrome, then the one or more JAK/STAT pathway variants are selected from the group consisting of JAK1 p. Y654F, JAK3 p. A573V, JAK3 p.Y980, JAK3 p. Y981, JAK3 p.S989I, STAT5B p.Y699, STAT5B p.N642H, and I12RG p.Y325.

13. The use of claim 10, wherein the determining comprises detecting variant JAK1, JAK3, STAT5B, and IL2RG nucleic acids or polypeptides, wherein the detecting variant JAK1, JAK3, STAT5B, and IL2RG nucleic acids comprises one or more nucleic acid detection method selected from the group consisting of sequencing, amplification and hybridization.

14. A method of determining a decreased time to adverse outcome in a human subject diagnosed with a mature T-cell leukemia, comprising:
a) contacting a biological sample from a human subject with a JAK/STAT pathway variant detection assay under conditions that the presence of a JAK/STAT pathway variant associated with a mature T-cell leukemia is determined, wherein the JAK/STAT pathway variant encodes a loss of function mutation and/or a gain of function mutuation, wherein the JAK/STAT pathway variant is one or more variants selected from JAK1, JAK3, STAT5B, and IL2RG; and
b) detecting a decreased time to adverse outcome in the human subject when the JAK/STAT pathway variants are present in the biological sample, wherein the adverse outcome is selected from the group consisting of relapse of the mature T-cell leukemia, metastasis, or death.

15. The method of claim 14,
wherein the JAK1 variant is one or more JAK1 mutations selected from the group consisting of JAK1 p.F636L, JAK1 p.G646C, JAK1 p. Y654F, JAK1 p.V658F, JAK1 p.G662W, JAK1 p.P664T, JAK1 p.S703I, and JAK1 p.T901R,
wherein the JAK3 variant is one or more JAK3 mutations selected from the group consisting of JAK3 p.ΔKNC563, JAK3 p.M511I, JAK3 p. A573V, JAK3 p.R657. JAK3 p.Y980, JAK3 p. Y981, and JAK3 p.S989I,
wherein the STAT5B variant is one or more STAT5B mutations selected from the group consisting of STAT5B p.T628S, STAT5B p.R659c, STAT5B p.Q706L, STAT5B p.N642H, STAT5B p.Y699, and STAT5B p.Y665H,
wherein the IL2RG variant is one or more IL2RG mutations selected from the group consisting of IL2RG p.ΔGSM268, IL2RG p.Y325, and IL2RG p. K315E.

16. The method of claim 14,
wherein if the mature T-cell leukemia is T-cell prolymphocytic leukemia, then the one or more JAK/STAT pathway variants are selected from the group consisting of JAK1 p.V658F, JAK1 p.S703I, JAK1 p.T901R, JAK3 p.ΔKNC563, JAK3 p.M511I, JAK3 p. A573V, JAK3 p.R657, STAT5B p.T628S, STAT5B p.R659c, STAT5B p.Q706L, STAT5B p.N642H, STAT5B p.Y665H, IL2RG p.ΔGSM268, and IL2RG p. K315E,
wherein if the mature T-cell leukemia is Sezary syndrome, then the one or more JAK/STAT pathway variants are selected from the group consisting of JAK1 p. Y654F, JAK3 p. A573V, JAK3 p.Y980, JAK3 p. Y981, JAK3 p.S989I, STAT5B p.Y699, STAT5B p.N642H, and I12RG p.Y325.

17. The method of claim 14, wherein the determining comprises detecting variant JAK1, JAK3, STAT5B, and IL2RG nucleic acids or polypeptides.

18. The method of claim 17, wherein the detecting variant JAK1, JAK3, STAT5B, and IL2RG nucleic acids comprises one or more nucleic acid detection method selected from the group consisting of sequencing, amplification and hybridization.
